(19) United States
(12) Patent Application Publication
(10) Pub. No.: US 2020/0283796 A1
(43) Pub. Date: Sep. 10, 2020

(54) DNA LAUNCHED RNA REPLICON SYSTEM (DREP) AND USES THEREOF

(71) Applicant: Massachusetts Institute of Technology, Cambridge, MA (US)

(72) Inventors: Ron Weiss, Newton, MA (US); Jin Huh, Watertown, MA (US); Jacob Becraft, Boston, MA (US)

(73) Assignee: Massachusetts Institute of Technology, Cambridge, MA (US)

(21) Appl. No.: 16/810,109

(22) Filed: Mar. 5, 2020

Related U.S. Application Data
(60) Provisional application No. 62/813,979, filed on Mar. 5, 2019.

(51) Int. Cl. C12N 15/85; C07K 16/00

(52) U.S. Cl. CPC C12N 15/85; C12N 2800/30; C12N 2830/50; C07K 16/00

(57) ABSTRACT

Provided herein, in some aspects, are antibody expression systems comprising DNA launched RNA replicons for high level antibody expression. In some embodiments, the antibody is a therapeutic antibody. In some embodiments, the antibody is an immune check point inhibitor. Methods of using the antibody expression system for treating diseases (e.g., cancer) are also provided.

### RELATED APPLICATION

This application claims priority under 35 U.S.C. § 119(e) to U.S. Provisional Application Ser. No. 62/813,979, filed Mar. 5, 2019, the entire contents of which are incorporated by reference herein.

### FEDERALLY SPONSORED RESEARCH

This invention was made with Government support under Grant Nos. R01 CA207029 and P50 GM098792 awarded by the National Institutes of Health, under Grant Nos. CCF1521925 and MCB1745645 awarded by the National Science Foundation, and under Grant No. W911NF-11-2-0054 awarded by the Army Research Office (ARO). The Government has certain rights in the invention

### BACKGROUND

Transgene expression directly influences efficacy of therapy. Prolonged, high level expression of transgenes is desired in therapeutic applications, e.g., vaccination and cancer immunotherapy. However it has been difficult to achieve such expression profile using traditional expression cassettes driven by pol-II promoters. For example, exogenous DNA may be epigenetically silenced and may also be present at lower-than-desired concentrations in cells due to limitations in transfection efficiencies in vivo. At the same time, the efficiency of current RNA delivery methods in vivo is significantly lower than their DNA counterparts.

### SUMMARY

Provided herein, in some aspects, are methods of encoding transgenes on RNA replicons, allowing fast and high-level expression of the transgenes. In some embodiments, the RNA replicon is a modified alphavirus. In some embodiments, the RNA replicons are encoded on DNA, yielding DNA launched RNA replicons (DREP). In some embodiments, DREP is encoded in a DNA virus (VREP, e.g., HSV-1). Encoding the RNA replicon on DNA allows fine-tuning the timing of the launch of RNA replicon using transcription and translational control. In some embodiments, such control is achieved by synthetic genetic circuits such as cell classifiers or cell classifiers.

In some embodiments, the DREP described herein is used to express more than one (e.g., 2, 3, 4, 5, or more) transgenes simultaneously. In some embodiments, two transgenes (a first transgene and a second transgene) are expressed simultaneously using the DREP. In some embodiments, the two transgenes are encoded on the same DREP. In some embodiments, the two transgenes are encoded on two different DREPs. In some embodiments, the expression level of the two transgenes may be adjusted such that the two transgenes are expressed at a desired ratio. In some embodiments, the expression levels of different transgenes are tuned by using different sub-genomic viral promoters.

For example, in some embodiments, a DREP used for expressing two transgenes comprises a promoter operably linked to a nucleic acid comprising a nucleotide sequence encoding one or more viral non-structural proteins, and comprising:

(a) a first subgenomic viral promoter operably linked to a nucleotide sequence encoding a first transgene; and

(b) a second subgenomic viral promoter operably linked to a nucleotide sequence encoding a second transgene.

In some embodiments, a DREP used for expressing two transgenes comprises:

(a) a promoter operably linked to a first nucleic acid comprising a nucleotide sequence encoding one or more viral non-structural proteins and a first subgenomic viral promoter operably linked to a nucleotide sequence encoding a first transgene; and

(b) a promoter operably linked to a second nucleic acid comprising a nucleotide sequence encoding one or more viral non-structural proteins and a second subgenomic viral promoter operably linked to a nucleotide sequence encoding a second transgene.

By using different sub-genomic viral promoters to control the expression level of the first transgene and the second transgene, the relative expression level of the two transgenes may be tuned and a desired expression ratio can be achieved.

Any transgene may be expressed using the DREP described herein. The transgenes may be, without limitation, proteins, peptides, or nucleic acids (e.g., DNA or RNA). In some embodiments, the transgenes are therapeutic molecules (e.g., therapeutic proteins or therapeutic RNAs). In some embodiments, the transgenes expressed using the DREP described herein are selected from enzymes, cytokines, chemokines, antigens, antibodies, and regulatory proteins.

In some embodiments, the transgenes that are expressed using the DREP described herein are immune modulators. In some embodiments, the immune modulator has anti-innate immunity activity. In some embodiments, the immune modulator is a cytokine (e.g., an anti-inflammatory cytokine such as, without limitation, IL-4, IL6, IL10, IL11, IL13, IL-1ra, and TGF-β).

In some embodiments, one or more of the transgenes expressed by the DREP described herein are selected from: GM-CSF, IFNg, IL15, CXCL10, CCL4, CD40L, secreted CD40L, IL12, MLKL and variants thereof (e.g., dominant active Q343A MLKL), Ubc12 and variants thereof (e.g., dominant negative mutants), scIL-27, secreted HMGB1, HMGB1, IKB super repressor, apoptin, pep-G3, RIPK3, Gasdermin D and variants thereof (e.g., GSDMD-NT mutant), Gasdermin E and variants thereof (e.g., GSDME-NT mutant), HSV-1 genes (e.g., without limitation, ICP4, ICP27, ICP0, VP16, gamma 34.5).

In some embodiments, one or more of the transgenes expressed by the DREP described herein are nucleic acid molecules (e.g., DNA or RNA). In some embodiments, one or more of the transgenes expressed by the DREP described herein are RNAi molecules (e.g., shRNA). In some embodiments, one or more of the transgenes expressed by the DREP described herein are mRNAs or fragments thereof.

In some embodiments, one or more of the transgenes expressed by the DREP described herein are components of antibodies (e.g., antibody heavy chain and light chain). In some aspects, the present disclosure provide methods of using the DREP to optimize the expression of antibodies by controlling the ratio between heavy chain and light chain expression using different subgenomic viral promoters. In some embodiments, the antibody is an immune checkpoint inhibitor. Methods of treating a disease (e.g., cancer) using the transgene (e.g., an antibody such as an immune checkpoint inhibitor) expressed by the DREP or VREP described herein are also provided.

Accordingly, some aspects of the present disclosure provide antibody expression systems containing a promoter operably linked to a nucleic acid comprising a nucleotide sequence encoding one or more viral non-structural proteins, and containing: (a) a first subgenomic viral promoter operably linked to a nucleotide sequence encoding an immunoglobulin heavy chain; and (b) a second subgenomic viral promoter operably linked to a nucleotide sequence encoding an immunoglobulin light chain.

Some aspects of the present disclosure provide antibody expression systems containing: (a) a promoter operably linked to a first nucleic acid containing a nucleotide sequence encoding one or more viral non-structural proteins and a first subgenomic viral promoter operably linked to a nucleotide sequence encoding an immunoglobulin heavy chain; and (b) a promoter operably linked to a second nucleic acid containing a nucleotide sequence encoding one or more viral non-structural proteins and a second subgenomic viral promoter operably linked to a nucleotide sequence encoding an immunoglobulin light chain.

In some embodiments, the promoter operably linked to the nucleic acid is a constitutive promoter. In some embodiments, the promoter of (a) and/or (b) is a constitutive promoter. In some embodiments, the promoter is a CMV promoter or a variant thereof. In some embodiments, the promoter is an inducible promoter. In some embodiments, the inducible promoter is activated by a signal produced from a cell classifier. In some embodiments, the inducible promoter is repressed by a signal produced from a cell classifier.

In some embodiments, (a) further contains a nucleotide sequence encoding a 3′ untranslated region (3′UTR) downstream of the nucleotide sequence encoding the immunoglobulin heavy chain. In some embodiments, (b) further contains a nucleotide sequence encoding a 3′ untranslated region (3′UTR) downstream of the nucleotide sequence encoding the immunoglobulin light chain.

In some embodiments, (a) further contains a poly-adenylation (polyA) signal sequence downstream of the 3′UTR. In some embodiments, (b) further contains a poly-adenylation (polyA) signal sequence downstream of the 3′UTR. In some embodiments, the polyA signal sequence of (a) contains a transcriptional terminator. In some embodiments, the polyA sequence of (b) contains a transcriptional terminator.

In some embodiments, the transcriptional terminator is selected from BGH_TT, antigenomic-BGH_TT, rb_glob_TT, and antigenomic_HD-SV40_TT.

In some embodiments, (a) further comprises a ribozyme located between the 3′UTR and the polyA signal, and/or (b) further comprises a ribozyme located between the 3′UTR and the polyA signal.

In some embodiments, the first viral subgenomic promoter is different from the second viral subgenomic promoter. In some embodiments, the first viral subgenomic promoter and the second viral subgenomic promoter lead to different expression levels of the heavy chain and the light chain. In some embodiments, the light chain and the heavy chain are expressed at a molar ratio of 1:1 (light chain:heavy chain) to 5:1 (light chain:heavy chain). In some embodiments, the light chain and the heavy chain are expressed at a molar ratio of 3:1 (light chain:heavy chain).

In some embodiments, (a) and/or (b) further contains a nucleotide sequence encoding one or more cleavage sites for an endoribonuclease. In some embodiments, the antibody expression further contains a promoter operably linked to a nucleotide sequence encoding an endoribonuclease that cleaves at the one or more cleavage sites. In some embodiments, the nuclease is selected from Csy4, Cse3, Cas6, Csy13, CasE, and variants thereof.

In some embodiments, the promoter operably linked to the nucleotide sequence encoding the nuclease is an inducible promoter. In some embodiments, the inducible promoter is regulated by a small molecule. In some embodiments, the small molecule is doxycycline or abscisic acid.

In some embodiments, the nucleotide sequence encoding the endoribonuclease is operably linked to a nucleotide sequence encoding a degradation signal. In some embodiments, the degradation signal is selected from: PEST, a destabilization domain from E. coli dihydrofolate reductase (ecDHFR), or a destabilization domain derived from human FKBP protein. In some embodiments, degradation of Csy4 mediated by the degradation signal is inhibited in the presence of TMP or 4-OHT.

In some embodiments, the one or more viral proteins are selected from: NSP 1-4.

In some embodiments, the immunoglobulin is an immunoglobulin G (IgG), IgM, IgA, IgD, or IgE. In some embodiments, the immunoglobulin is an immune checkpoint inhibitor. In some embodiments, the immune checkpoint inhibitor is selected from: anti-CTLA4, anti-PD1, and anti-PD-L1. In some embodiments, the immune checkpoint inhibitor is anti-CTLA4.

In some embodiments, the antibody expression system is one or more engineered viral genomes. In some embodiments, the viral genome is the genome of an oncolytic virus. In some embodiments, the oncolytic virus is selected from the group consisting of: alphaviruses, adenoviruses, reoviruses, measles virus, herpes simplex virus, Newcastle disease virus and vaccinia virus. In some embodiments, the oncolytic virus is herpes simplex virus 1 (HSV-1).

In some embodiments, the antibody expression system is one or more Minicircle DNA molecules.

Other aspects of the present disclosure provide viral particles or cells containing the antibody expression system described herein. In some embodiments, the cell is a diseased cell. In some embodiments, the diseased cell is a cancer cell. In some embodiments, the cell is a healthy cell. In some embodiments, the cell is an immune cell.

Further provided herein are methods of expressing an immunoglobulin, containing delivering the antibody expression system or the viral particle described herein to a cell and culturing the cell under conditions that allow expression of the light chain and the heavy chain. In some embodiments, the promoter operably linked to the nucleotide sequence encoding one or more viral non-structural proteins is an inducible promoter, and the method further contains providing an inducer that activates the promoter.

In some embodiments, the cell is in vitro. In some embodiments, the cell is ex vivo. In some embodiments, the cell is in vivo. In some embodiments, the cell is a diseased cell, a healthy cell, or an immune cell. In some embodiments, the diseased cell is a cancer cell. In some embodiments, the cell is a healthy cell. In some embodiments, the cell is an immune cell.

Further provided herein are methods of treating a disease, containing administering to a subject in need thereof an effective amount of the antibody expression system or the viral particle described herein. In some embodiments, the disease is cancer. In some embodiments, the therapeutic immunoglobulin is an immune checkpoint inhibitor. In some embodiments, the immune checkpoint inhibitor is selected from: anti-CTLA4, anti-PD1, anti-PD-L1.

Other aspects of the present disclosure provide compositions containing the antibody expression system or the viral particle described herein. In some embodiments, the composition further contains a pharmaceutically acceptable carrier.

Yet other aspects of the present disclosure provide methods of producing the antibody expression system, containing:

(i) providing a plurality of genetic elements containing a plurality of viral subgenomic promoters, a nucleotide sequence encoding an immunoglobulin heavy chain, a nucleotide sequence encoding an immunoglobulin light chain, and optionally a nucleotide sequence encoding a 3′ untranslated region (3′UTR), wherein each genetic element is flanked at the 3′ end and the 5′ end by a recognition and cleavage site for a first type IIS restriction endonuclease, and wherein the recognition and cleavage site is engineered to allow directional assembly of the plurality genetic elements;

(ii) assembling a first transcriptional unit containing, in order from 5′ to 3′, a first subgenomic promoter, the nucleotide sequence encoding the immunoglobulin heavy chain, and optionally the nucleotide sequence encoding the 3′UTR, by combining the genetic elements with:
(a) the first type IIS restriction endonuclease;
(b) a ligase; and
(c) a first destination vector containing a pair of the recognition and cleavage sites for the first type IIS restriction endonuclease and a pair of the recognition and cleavage sites for a second type IIS restriction endonuclease, wherein the pair of recognition and cleavage sites for the second type IIS restriction endonuclease enclose the pair of recognition and cleavage sites for the first type IIS restriction endonuclease, and wherein the two pairs of recognition and cleavage sites are positioned in inverse orientation relative to each other;
wherein the contacting is carried out under conditions that allow the cleavage at the recognition and cleavage sites for the first type IIS restriction endonuclease and the ligation of resulting fragments in a directional manner;

(iii) assembling a second transcriptional unit containing, in order from 5′ to 3′, a second subgenomic promoter, the nucleotide sequence encoding the immunoglobulin light chain, and optionally the nucleotide sequence encoding the 3′UTR, by contacting the genetic elements with:
(a) the first type IIS restriction endonuclease;
(b) a ligase; and
(c) a first destination vector containing a pair of the recognition and cleavage sites for the first type IIS restriction endonuclease and a pair of the recognition and cleavage sites for a second type IIS restriction endonuclease, wherein the pair of recognition and cleavage sites for the second type IIS restriction endonuclease enclose the pair of recognition and cleavage sites for the first type IIS restriction endonuclease, and wherein the two pairs of recognition and cleavage sites are positioned in inverse orientation relative to each other;
wherein the contacting is carried out under conditions that allow the cleavage at the recognition and cleavage sites for the first type IIS restriction endonuclease and the ligation of resulting fragments in a directional matter;

(iv) assembling the antibody expression system by combining the first transcriptional unit obtained in (ii) and the second transcriptional unit obtained in (iii) with:
(a) the second type IIS restriction endonuclease;
(b) a ligase; and
(c) a second destination vector containing a promoter operably linked to a nucleotide sequence encoding one or more viral non-structural proteins, and a pair of the recognition and cleavage sites for the second type IIS restriction endonuclease,
wherein the contacting is carried out under conditions that allow the cleavage at the recognition and cleavage sites for the second type IIS restriction endonuclease and the ligation of resulting fragments in a directional manner.

In some embodiments, the first type IIS restriction endonuclease is BsaI. In some embodiments, the second type IIS restriction endonuclease is SapI. In some embodiments, the immunoglobulin is an immune checkpoint inhibitor.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

The accompanying drawings are not intended to be drawn to scale. For purposes of clarity, not every component may be labeled in every drawing.

FIG. 1: Schematic of DREP packaged into HSV-1 (VREP). DREP is encoded in HSV-1 genome and packaged into virions. Upon transcription of DREP, RNA replicon consisted of 5′ Cap, nsP1-4, heterologous protein, 3′ UTR, and polyA is generated and self-replicated. Subgenomic RNA is then produced from subgenomic promoter and effector protein is translated.

FIGS. 2A-2D: VREP expression on day 10. (FIG. 2A) Graph showing 1:10 packaging supernatant HSV-DREP. (FIG. 2B) Graph showing 1:10 packaging pellet HSV-DREP. (FIG. 2C) Graph showing 1:50 packaging pellet HSV-DREP. (FIG. 2D) Graph showing 1:10 packaging pellet YFP-HSV control. The graphs show prolonged and high expression compared to traditional (YFP) HSV-1 even with a low titer.

FIGS. 3A-3C: Microscopy images showing expression of HSV-DREP on day 10. VREP is found in daughter cells that form positive colonies. The images show prolonged transgene (YFP) expressions compared to traditional HSV-1. (FIG. 3A) Microscopy of 1:10 packaging supernatant. (FIG. 3B) Microscopy of 1:10 packaging pellet HSV-DREP. (FIG. 3C) Microscopy of 1:50 packaging pellet HSV-DREP.

FIGS. 4A-4B: HSV-1 launched RNA replicon system (VREP) for prolonged and high level expression of transgenes with high precision controls. (FIG. 4A) Schematic showing a cell classifier which computes levels of multiple miRNAs and launches RNA replicon encoding transgenes only if the computation is correct. (FIG. 4B) Graphs showing performance of the cell classifier variants with DREP in HEK (Off-state) and VERO (On-state) by measuring mKate expressed from DREP.

FIG. 5: Replicon expression from a DNA plasmid. The top schematic shows the transcription, amplification, and RNA-dependent transcription of DREP, whose launch is under control of a CMV promoter. The ribozyme at the 3′ end is used to make a poly-A tail without a transcriptional terminator. The initial RNA transcript is self-replicating. The bottom left graphs show standard plasmid and DREP expressing mKate plotted against EBFP2. 15 fmol of hEF1A_EBFP2 plasmid was used as transfection control. The graphs show high expression even at low transfection levels.

FIG. 6: Schematic of optimization of DREP regarding CMV positioning and termination sequence. Three variants of CMV promoter and four variants of polyA (transcription termination) sequences were tested.

FIGS. 7A-7E: Histograms of DREP with different CMV promoters and transcription termination sequences. CMV 1 with HDV-BGH and CMV 1 with HDV-SV40 shows a desired expression profile. (FIG. 7A) CMV 1 with HDV-BGH (FIG. 7B) CMV 2 with HDV-BGH. (FIG. 7C) CMV 3 with HDV-BGH. (FIG. 7D CMV 1 with BGH. (FIG. 7E) CMV 1 with HDV-SV40.

FIGS. 8A-8E: Comparison of expression profiles of DREP, RNA replicon, and plasmid DNA in HEK293 cells. DREP and RNA replicon exhibit high expression of mKate compared to that of plasmid DNA. (FIG. 8A) Microscopy image showing DREP expression and a histogram of mKate expression after 48 hours. (FIG. 8B) Microscopy image showing RNA-replicon expression and a histogram of mKate expression after 48 hours. (FIG. 8C) Microscopy image showing DNA (hCMV-mKate) expression and a histogram of mKate expression after 48 hours. (FIG. 8D) Graph showing expression profiles in HEK293 over time based on mKate signal (FU). (FIG. 8E) Graph showing expression profiles in HEK293 over time based percentage of cells expressing mKate (%).

FIGS. 9A-9E: Comparison of expression profiles of DREP, RNA replicon, and plasmid DNA in CHO-K1. (FIG. 9A) Microscopy image showing DREP expression and a histogram of mKate expression after 48 hours. (FIG. 9B) Microscopy image showing RNA-replicon expression and a histogram of mKate expression after 48 hours. (FIG. 9C) Microscopy image showing DNA (hCMV-mKate) expression and a histogram of mKate expression after 48 hours. (FIG. 9D) Graph showing expression profiles in CHO-K1 over time based on mKate signal (FU). (FIG. 9E) Graph showing expression profiles in CHO-K1 over time based percentage of cells expressing mKate (%).

FIGS. 10A-10D: Graphs of plasmid DNA and DREP expression. Either plasmid DNA or DREP is co-transfected with a plasmid expressing EBFP2. Plasmid copy number is estimated by EBFP2 signal. DREP shows high expression profile even when plasmid copy number in a given cell is low (FIG. 10A) Graphs of plasmid expression using different amounts of plasmid. (FIG. 10B) Graphs of DREP expression using different amounts of plasmid. (FIG. 10C) mKate expression from DREP vs. plasmid EBFP2 (MEFL) in given plasmid copy number. (FIG. 10D) Mean mKate fluorescence with varied amounts of DNA.

FIG. 11: Rapid assembly of DREP cassettes. Schematic of VEE replicon MoClo assembly strategy. Each transcription unit was divided into three parts: a sub-genomic promoter (SGP), open reading frame (ORF), and 3′-untranslated region (3′UTR). They are then assembled into a complete DREP by another layer of MoClo reaction.

FIG. 12: Characterization of two SGP replicons. All combinations of chosen low (SGP5), midrange (SGP30), and high (SGP15) expressing SGPs with and without an additional 3′UTR were generated using replicon MoClo assembly to determine the expression control possible using only replicon sequence elements. Fluorescence was normalized by single SGP replicons expressing either mVenus or mKate under SGP30.

FIGS. 13A-13B: Characterization of three SGP replicons. (FIG. 13A) Schematic showing SGP1, SGP2, and SGP3 regions. (FIG. 13B) Graphs showing quantification of low, medium and high mKate in the presence of various mVenus and EBFP concentrations.

FIG. 14: Minicircle (mc) DNA technology interface with replicon. Minicircles allow for the delivery of plasmid DNA expression cassettes free of bacterial sequence (unique to DNA platforms). The duration is prolonged and delivery is easier due to size (relative to counter DNA vectors). This technology may be able to be applied in combination with the replicon to achieve prolonged and extremely high levels of expression, effective expression without highly efficient delivery, and enable novel regulatory mechanisms.

FIG. 15: Expression of minicircle (mc) and mcDREP constructs. Mc plasmids expressing mKate fluorescent protein were grown in ZYCY10P3S2T producer E. coli strain (System Biosciences) and induced with arabinose (0.1%, 5 hours at 30° C.). Mc plasmid was purified by gel extraction and mcDREP plasmid was purified by incubation with I-SceI and exoV nucleases. Prnt, parental mc plasmid; Ind, crude mixture after arabinose induction.

FIGS. 16A-16B: mc and mcDREP (parental and excised) expression in HEK293a cells. HEK293a cells (ATCC) were co-transfected with parental mc, mcDNA, DREP or mcDREP (15 fmol) and EBFP expressing plasmid (200 ng) using viafect transfection reagent. Cells were cultured in DMEM media with 10% FBS for 1 week. At 24 hours, 72 hours and 1 week post-transfection cells were assayed by FACS for mKate expression. (FIG. 16A, top panel) Fluorescent cell images of cells 48 hours post-transfection (Texas-Red filter; EVOS cell imaging system, Life Technology). (FIG. 16A, middle panel) FACS analysis of cells 48 hours post-transfection, from which the percentage of positive cells (P4 gate) and their mean fluorescence (PE-Texas-Red channel) was calculated. (FIG. 16A, bottom panel) FACS analysis of mKate and EBFP expression. (FIG. 16B) Mean fluorescence of mKate-positive cells (FU, fluorescence units) and percentage of positive cells (P4 gate) derived from FACS analysis.

FIG. 17: Fluorescent images of HEK293a transfected cells. HEK293a cells (ATCC) were co-transfected with parental mc and DREP or mcDREP at a range of decreasing concentrations and a fixed concentration of EBFP transfection marker (200 ng) using viafect lipid reagent. Fluorescent cell images were taken 48 hours post-transfection (Texas-Red and DAPI filters; EVOS cell imaging system, Life Technology).

FIGS. 18A-18B: FACS analysis of mKate and EBFP levels. (FIG. 18A) HEK293a cells (ATCC) were co-transfected with parental mc and DREP or mcDREP at a range of decreasing concentrations and a fixed concentration of EBFP transfection marker (200 ng) using viafect lipid reagent. FACS analysis was performed 48 hours post-transfection, from which the percentage of positive cells and their mean red and blue fluorescence was calculated. (FIG. 18B) Data was plotted as mKate expression (mean fluorescence intensity) of mKate-expressing cells normalized by transfection marker (the ratio of mKAte-positive to EBFP-positive cells).

FIG. 19: Implementation of DNA minicircle launched replicons. The replicon has been integrated as the expressed transgene from the minicircle in several configurations: one as two separate replicons each expressing a chain of the antibody independently, another as a single minicircle with the chains being cleaved by a viral 2A tag, and another as a single minicircle with a two unit replicon with a chain expressed from each ORF. This allows the technologies from previous work to be adapted to finely tune AB expression.

FIG. 20: DREP Ab expression at low delivery. Replicon technologies allow for the efficient expression of anti-HA antibodies (s139) under low delivery conditions. Traditional minicircles are viable only when co-delivery is high. The replicon based minicircles drastically outperform when delivery is low or inefficient.

FIG. 21: Optimization of antibody expression: ratio tuning leveraged to increase antibody expression. Using the SGP scanning technology, a wide range of heavy chain (HC) and light chain (LC) ratios can be surveyed. This allows for dial in expression, which is indifferent to issues surround delivery or variability of an individual.

FIGS. 22A-22C: Regulating DREP expression in plasmid. (FIG. 22A) Schematic showing DREP under the control of a TRE-tight promoter. (FIG. 22B) Graph showing the percentage of cells expressing mKate in no DREP, CMV_DREP and TRE-t_DREP environments. (FIG. 22C) Graphs showing −rtTA, −Dox in the presence of EBFB2 and mKate, and +rtTA and +Dox in the presence of EBFP2 and mKate. A reasonably tight “off” state was observed. Less than 20% of cells showed promoter leakiness, but any leakiness resulted in full expression. The X-axis shows the combination of the two SGPs used. The gene under control of the first SGP is followed by 3′UTR and the gene under control of the second SGP is followed by 3′UTR and polyA. The SGPs used in this figure (SGP5, SGP15, and SGP30) are provided in Table 3.

FIGS. 23A-23C: Expression of mKate2 from DNA-launched replicon (DREP) and regulation by Csy4. (FIG. 23A) Schematic showing RNA replication and a graph showing Csy4 regulation of Replicon. (FIG. 23B) Schematic showing expression of mKate2 from DREP. (FIG. 23C) Graphs showing Csy4 effectively represses expression from replicon. This is useful for DREP regulation as it can express from a separate transcription unit and has tighter control over Csy4.

FIGS. 24A-24B: Optimization of DREP regulation by small molecule induced Csy4 expression. (FIG. 24A) Schematic showing DREP regulation by CYS4 expression. Inducible promoters or DD-tags were used to control Csy4 expression. (FIG. 24B) Graphs showing DREP/Cys4 concentration in drug and no drug environments.

FIGS. 25A-25B: Transfection of Csy4 Suppresses DNA-launched replicon bearing Csy4Rec. (FIG. 25A) Graph showing EBFP2 transfection marker (MEBFP) per mean mVenus-PEST from DREP (MEFL) of Csy4 and no Csy4. Constitutive CMV-Cys4 were co-transfected with hEF1a-EBFP2 marker (1:10 ratio Csy4-to-EBFP2) into CHO-LP cells bearing integrated DREP expressing mVenus-PEST from a subgenomic promoter. (FIG. 25B) Graph showing mVenus-PEST from DREP (MEFL) per density of Csy4 and no Csy4. Cells transfected with marker alone (No Csy4) express mVenus-PEST in a stochastic, bimodal all-or-nothing fashion, but Csy4 transfection suppresses expression of mVenus-PEST.

FIGS. 26A-26D: Evaluation of DREP in Engineered HSV-1 genome. FIG. 26A is a schematic diagram of the engineered HSV-1 genome (MD306) having a landing pad (LP1) at which location the DREP or negative control was integrated. FIG. 26B are schematic designs of a negative control (CMV promoter driving mKate expression; CMV-mKate) and DREP (CMV promoter, 5′UTR, nSP1-4, subgenomic promoter (SGP) driving mKate expression, 3′UTR, ribozyme, and polyA; DREP-mKate). The negative control and the DREP were integrated to the HSV-1 genome (MD306) at LP1, respectively. FIG. 26C is a graph showing doubling time of HSV-1 carrying either CMV-mKate or DREP-mKate in Vero, A549 and HT-29 cells. FIG. 26D is a graph showing mKate expression level by CMV-mKate and DREP-mKate in HSV-1 genome.

FIGS. 27A-27B: In vivo validation of the HSV-1-DREP-mCherry construct. mKate in FIG. 26B was replaced with mCherry. FIG. 27A is a graph showing mCherry expression in tumor cells isolated from each mouse infected with HSV-1-DREP-mCherry or HSV-1-CMV-mCherry. FIG. 27B shows the average mCherry expression level in tumor cells harvested mice infected with HSV-1-DREP-mCherry or HSV-1-CMV-mCherry.

FIGS. 28A-28C: In vivo validation of the HSV-1-DREP-cytokine (e.g., GM-CSF) construct. mKate in FIG. 26B was replaced with GM-CSF. FIG. 28A is a graph showing GM-CSF expression by HSV-1-CMV-GM-CSF and HSV-1-DREP-GM-CSF in 4T1 tumor cells in vivo 1 day after infection. FIG. 28B is a graph showing GM-CSF expression by HSV-1-CMV-GM-CSF and HSV-1-DREP-GM-CSF in 4T1 tumor cells in vivo 3 day after infection in log scale. FIG. 28C is a graph showing GM-CSF expression by HSV-1-CMV-GM-CSF and HSV-1-DREP-GM-CSF in 4T1 tumor cells in vivo 3 day after infection in linear scale.

FIGS. 29A-29B: In vitro and in vivo validation of cytokine expression by DREP in additional tumor models. FIG. 29A is a graph showing GM-CSF expression by HSV-1-CMV-GM-CSF and HSV-1-DREP-GM-CSF in cancer cell line supernatants in vitro (in each group, the left bar represents PBS+10% glycerol; the middle bar represents HSV-1-CMV-GM-CSF; and the right represents HSV-1-DREP-GM-CSF). FIG. 29B is a graph showing GM-CSF expression in mice graphed with cancer cell lines and infected with HSV-1-CMV-GM-CSF and HSV-1-DREP-GM-CSF (in each group, the left bar represents PBS+10% glycerol; the middle bar represents HSV-1-CMV-GM-CSF; and the right represents HSV-1-DREP-GM-CSF).

### DETAILED DESCRIPTION OF CERTAIN EMBODIMENTS

Described herein, in some aspects, are DNA launched replicon (DREP) systems for transgene expression. In some embodiments, the DREP can be integrated into the genome of a virus to encode a viral replicon (VREP) that encode the transgene. The DREP RNA replicon that contains components required for the transgene expression. The compositions and methods described herein can be used to obtain very high levels of transgenes. The DREP/VREP system described herein can be used to express any one or more (e.g., 2, 3, 4, 5, 6, 7, 8, 9, 10, or more) transgenes. The transgene can be any molecule that is of interest to those skilled in the art, e.g., nucleic acids or proteins. In some embodiments, the nucleic acid is a DNA or RNA (e.g., a RNAi molecule). In some embodiments, the protein is a therapeutic protein, e.g., without limitation, antigens, antibodies, enzymes, regulatory proteins, immunomodulators, cytokines, and chemokines.

In some embodiments, the transgenes that are expressed using the DREP described herein are immune modulators. In some embodiments, the immune modulator has anti-innate immunity activity. In some embodiments, the immune modulator is a cytokine (e.g., an anti-inflammatory cytokine such as, without limitation, IL-4, IL6, IL10, IL11, IL13, IL-1ra, and TGF-β).

In some embodiments, one or more of the transgenes expressed by the DREP described herein are selected from: GM-CSF, IFNg, IL15, CXCL10, CCL4, CD40L, secreted CD40L, IL12, MLKL and variants thereof (e.g., dominant active Q343A MLKL), Ubc12 and variants thereof (e.g., dominant negative mutants), scIL-27, secreted HMGB1, HMGB1, IKB super repressor, apoptin, pep-G3, RIPK3, Gasdermin D and variants thereof (e.g., GSDMD-NT mutant), Gasdermin E and variants thereof (e.g., GSDME-NT mutant), HSV-1 genes (e.g., without limitation, ICP4, ICP27, ICP0, VP16, gamma 34.5). In some embodiments, one or more of the transgenes expressed by the DREP described herein are components of antibodies (e.g., antibody heavy chain and light chain).

In some embodiments, one or more of the transgenes expressed by the DREP described herein are nucleic acid molecules (e.g., DNA or RNA). In some embodiments, one or more of the transgenes expressed by the DREP described herein are RNAi molecules (e.g., shRNA). In some embodiments, one or more of the transgenes expressed by the DREP described herein are mRNAs or fragments thereof.

In some aspects, the present disclosure provide methods of using the DREP system as an antibody expression system. An “antibody expression system” refers to one or more nucleic acids that recombinantly express the antibody or components of the antibody (e.g., heavy chain and/or light chain, or an antigen-binding fragment). The nucleic acids in the antibody expression system described herein can be engineered (e.g., via the use of different subgenomic viral promoters) such that different components of the antibody express at different levels and the expression of the whole and functional antibody is optimized. Other mechanisms of regulating the expression level of the transgene are also provided, e.g., by an endoribonuclease that can degrade the RNA replicon. In some embodiments, the endoribonuclease itself can be regulated by degradation signals and/or small molecule inducers, further providing fine-tuning power to the expression level of the transgene.

An “antibody” or “immunoglobulin (Ig)” is a large, Y-shaped protein produced mainly by plasma cells that is used by the immune system to neutralize an exogenous substance (e.g., a pathogens such as bacteria and viruses). Antibodies are classified as IgA, IgD, IgE, IgG, and IgM. “Antibodies” and “antibody fragments” include whole antibodies and any antigen binding fragment (i.e., “antigen-binding portion”) or single chain thereof. In some embodiments, an antibody is a glycoprotein comprising two or more heavy (H) chains and two or more light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody may be a polyclonal antibody or a monoclonal antibody.

The basic 4-chain antibody unit is a heterotetrameric glycoprotein composed of two identical L chains and two H chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, (e.g., Basic and Clinical Immunology, 8th edition, Daniel P. Stites, Abba I. Ten and Tristram G. Parslow (eds.), Appleton & Lange, Norwalk, Conn., 1994, page 71 and Chapter 6, incorporated herein by reference).

In some embodiments, the antibody expressed using the antibody expression system described herein is a monoclonal antibody. A “monoclonal antibody” is an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies.

In some embodiments, the monoclonal antibodies described herein encompass “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc.), and human constant region sequences.

In some embodiments, the antibody expressed using the antibody expression system described herein is a polyclonal antibody. A “polyclonal antibody” is a mixture of different antibody molecules which react with more than one immunogenic determinant of an antigen. Polyclonal antibodies may be isolated or purified from mammalian blood, secretions, or other fluids, or from eggs. Polyclonal antibodies may also be recombinant. A recombinant polyclonal antibody is a polyclonal antibody generated by the use of recombinant technologies. Recombinantly generated polyclonal antibodies usually contain a high concentration of different antibody molecules, all or a majority of (e.g., more than 80%, more than 85%, more than 90%, more than 95%, more than 99%, or more) which are displaying a desired binding activity towards an antigen composed of more than one epitope.

In some embodiments, the antibody expressed using the antibody expression system described herein are “humanized” for use in human (e.g., as therapeutics). “Humanized” forms of non-human (e.g., rodent) antibodies are chimeric antibodies that contain minimal sequence derived from the non-human antibody. Humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

In some embodiments, an antibody fragment containing the antigen-binding portion of an antibody can be expressed using the antibody expression system described herein. The antigen-binding portion of an antibody refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (e.g., as described in Ward et al., (1989) Nature 341:544-546, incorporated herein by reference), which consists of a VH domain; or a NANOBODY®, such as a VH domain of a camelid (VHH), a humanized VHH domain, or a camelized VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883, incorporated herein by reference). Such single chain antibodies are also intended to be encompassed within the term “antigen-binding portion” of an antibody. These antibody fragments are obtained using conventional techniques known to those with skill in the art, and the fragments are screened for utility in the same manner as are full-length antibodies.

In some embodiments, an antibody fragment may be a Fc fragment, a Fv fragment, a single-chain Fv fragment, or a single domain antibody. The Fc fragment comprises the carboxy-terminal portions of both H chains held together by disulfides. The effector functions of antibodies are determined by sequences in the Fc region, which region is also the part recognized by Fc receptors (FcR) found on certain types of cells. In some embodiments, the antibody expression system can be used to express two or more different antibody fragments, such as two or more scFvs.

The Fv fragment is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

Any immunoglobulin (e.g., IgG, IgM, IgA, IgD, and IgE) may be produced using the antibody expression system described herein. Any antibody may be produced using the antibody expression system described herein. Non-limiting examples of antibodies and fragments thereof include: bevacizumab (AVASTIN®), trastuzumab (HERCEPTIN®), alemtuzumab (CAMPATH®, indicated for B cell chronic lymphocytic leukemia), gemtuzumab (MYLOTARG®, hP67.6, anti-CD33, indicated for leukemia such as acute myeloid leukemia), rituximab (RITUXAN®), tositumomab (BEXXAR®, anti-CD20, indicated for B cell malignancy), MDX-210 (bispecific antibody that binds simultaneously to HER-2/neu oncogene protein product and type I Fc receptors for immunoglobulin G (IgG) (Fc gamma RI)), oregovomab (OVAREX®, indicated for ovarian cancer), edrecolomab (PANOREX®), daclizumab (ZENAPAX®), palivizumab (SYNAGIS®, indicated for respiratory conditions such as RSV infection), ibritumomab tiuxetan (ZEVALIN®, indicated for Non-Hodgkin's lymphoma), cetuximab (ERBITUX®), MDX-447, MDX-22, MDX-220 (anti-TAG-72), IOR-05, IOR-T6 (anti-CD1), IOR EGF/R3, celogovab (ONCOSCINT® OV103), epratuzumab (LYMPHOCIDE®), pemtumomab (THERAGYN®), Gliomab-H (indicated for brain cancer, melanoma), anti-Isocitrate Dehydrogenase 1 (IDH1), anti-Cbl Proto-Oncogene B (CBLB) and anti-Cytokine-inducible SH2-containing protein (CISH).

In some embodiments, the antibody is an antibody that inhibits an immune check point protein (termed herein as an “immune checkpoint inhibitor”). An “immune checkpoint” is a protein in the immune system that either enhances an immune response signal (co-stimulatory molecules) or reduces an immune response signal. Many cancers protect themselves from the immune system by exploiting the inhibitory immune checkpoint proteins to inhibit the T cell signal. Exemplary inhibitory checkpoint proteins include, without limitation, Cytotoxic T-Lymphocyte-Associated protein 4 (CTLA-4), Programmed Death 1 receptor (PD-1), T-cell Immunoglobulin domain and Mucin domain 3 (TIM3), Lymphocyte Activation Gene-3 (LAG3), V-set domain-containing T-cell activation inhibitor 1 (VTVN1 or B7-H4), Cluster of Differentiation 276 (CD276 or B7-H3), B and T Lymphocyte Attenuator (BTLA), Galectin-9 (GALS), Checkpoint kinase 1 (Chk1), Adenosine A2A receptor (A2aR), Indoleamine 2,3-dioxygenase (IDO), Killer-cell Immunoglobulin-like Receptor (KIR), Lymphocyte Activation Gene-3 (LAG3), and V-domain Ig suppressor of T cell activation (VISTA).

Some of these immune checkpoint proteins need their cognate binding partners, or ligands, for their immune inhibitory activity. For example, A2AR is the receptor of adenosine A2A and binding of A2A to A2AR activates a negative immune feedback loop. As another example, PD-1 associates with its two ligands, PD-L1 and PD-L2, to down regulate the immune system by preventing the activation of T-cells. PD-1 promotes the programmed cell death of antigen specific T-cells in lymph nodes and simultaneously reduces programmed cell death of suppressor T cells, thus achieving its immune inhibitory function. As yet another example, CTLA-4 is present on the surface of T cells, and when bound to its binding partner CD80 or CD86 on the surface of antigen-present cells (APCs), it transmits an inhibitory signal to T cells, thereby reducing the immune response.

The immune checkpoint inhibitors that may be expressed using the antibody-expression system described herein may inhibit the binding of the immune checkpoint protein to its cognate binding partner, e.g., PD-1, CTLA-4, or A2aR. In some embodiments, the immune checkpoint inhibit is selected from anti-CTLA-4, anti-PD-1, anti-PD-L1, anti-TIM3, anti-LAG3, anti-B7-H3, anti-B7-H4, anti-BTLA, anti-GALS, anti-Chk, anti-A2aR, anti-IDO, anti-KIR, anti-LAG3, anti-VISTA antibody, or a combination of any two or more of the foregoing antibodies.

Examples of monoclonal antibodies that are immunecheckpoint inhibitors approved by the FDA for cancer therapy, and can be produced using the antibody expression system described herein include, without limitation: Rituximab (available as Rituxan™) Trastuzumab (available as Herceptin™), Alemtuzumab (available as Campath-IH™) Cetuximab (available as Erbitux™), Bevacizumab (available as Avastin™), Panitumumab (available as Vectibix™), Gemtuzumab ozogamicin (available as Mylotarg™), Ibritumomab tiuxetan (available as Zevalin™), Tositumomab (available as Bexxar™), Ipilimumab (available as Yervoy™), Ofatunumab (available as Arzerra™), Daclizumab (available as Zinbryta™), Nivolumab (available as Opdivo™), and Pembrolizumab (available as Keytruda™). Examples of monoclonal antibody immune checkpoint inhibitors currently undergoing human clinical testing for cancer therapy in the United States include, without limitation: WX-G250 (available as Rencarex™), Zanolimumab (available as HuMax-CD4), ch14.18, Zalutumumab (available as HuMax-EGFr), Oregovomab (available as B43.13, OvalRex™), Edrecolomab (available as IGN-101, Panorex™), 131I-chTNT-I/B (available as Cotara™), Pemtumomab (available as R-1549, Theragyn™), Lintuzumab (available as SGN-33), Labetuzumab (available as hMN14, CEAcide™), Catumaxomab (available as Removab™), CNTO 328 (available as cCLB8), 3F8, 177Lu-J591, Nimotuzumab, SGN-30, Ticilimumab (available as CP-675206), Epratuzumab (available as hLL2, LymphoCide™) 90Y-Epratuzumab, Galiximab (available as IDEC-114), MDX-060, CT-011, CS-1008, SGN-40, Mapatumumab (available as TRM-I), Apolizumab (available as HuID10, Remitogen™) and Volociximab (available as M200).

In some embodiments, the immune checkpoint inhibitor is an anti-CTLA-4 antibody. In some embodiments, the anti-CTLA-4 antibody is ipilimumab (Yervoy®). In some embodiments, the immune checkpoint inhibitor is an anti-PD-1 antibody is pembrolizumab (Keytruda®) or nivolumab (Opdivo®).

The antibody expression system described in comprises one or more nucleic acids comprising a promoter operably linked to a nucleotide sequence encoding a RNA replicon. The RNA replicon comprises nucleotide sequences encoding viral non-structural promoters, subgenomic viral promoters, and nucleotide sequences encoding the antibody heavy chain and/or light chain. In some embodiments, the promoter is a constitutive promoter. In some embodiments, the promoter is an inducible promoter.

The promoter operably linked to the nucleic acid is used to launch RNA replicon from the DNA molecule. In some embodiments, the promoter is a constitutive promoter. Any constitutive promoters described herein or known in the art may be used to launch the RNA replicon (e.g., a CMV promoter or a variant thereof).

In some embodiments, the constitutive promoter is an enhancer linked to a minimal CMV promoter. An “enhancer,” as used herein, refers to a transcriptional enhancer. The terms “enhancer” and “transcriptional enhancer” are used interchangeably herein. An enhancer is a short (50-1500 bp) region of DNA that can be bound by activators to increase the likelihood that transcription of a particular gene will occur. Enhancers are cis-acting and can be located up to 1 Mbp (1,000,000 bp) away from the gene, upstream or downstream from the transcription start site. Enhancers are found both in prokaryotes and eukaryotes. There are hundreds of thousands of enhancers in the human genome. Such constitutive promoters are described in the art, e.g., in Schlabach et al., PNAS Feb. 9, 2010 107 (6) 2538-2543, incorporated herein by reference.

In some embodiments, the promoter is an inducible promoter. Any known inducible promoters described herein and are known in the art may be used. When an inducible promoter is used to launch the RNA replicon, an inducer that activates the inducible promoter can be added at a time when transgene expression is desired. The inducible can also be removed when no transgene expression is desired, such that temporary expression of the transgene is achieved.

A “promoter” refers to a control region of a nucleic acid sequence at which initiation and rate of transcription of the remainder of a nucleic acid sequence are controlled. A promoter drives expression or drives transcription of the nucleic acid sequence that it regulates. A promoter may also contain sub-regions at which regulatory proteins and molecules may bind, such as RNA polymerase and other transcription factors. Promoters may be constitutive, inducible, activatable, repressible, tissue-specific or any combination thereof. A promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see U.S. Pat. Nos. 4,683,202 and 5,928,906).

An “inducible promoter” refer to a promoter that is characterized by regulating (e.g., initiating or activating) transcriptional activity when in the presence of, influenced by or contacted by an inducer signal. An inducer signal may be endogenous or a normally exogenous condition (e.g., light), compound (e.g., chemical or non-chemical compound) or protein that contacts an inducible promoter in such a way as to be active in regulating transcriptional activity from the inducible promoter. Thus, a “signal that regulates transcription” of a nucleic acid refers to an inducer signal that acts on an inducible promoter. A signal that regulates transcription may activate or inactivate transcription, depending on the regulatory system used. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter. In some embodiments, using inducible promoters in the genetic circuits results in the conditional expression or a “delayed” expression of a gene product.

The administration or removal of an inducer signal results in a switch between activation and inactivation of the transcription of the operably linked nucleic acid sequence. Thus, the active state of a promoter operably linked to a nucleic acid sequence refers to the state when the promoter is actively regulating transcription of the nucleic acid sequence (i.e., the linked nucleic acid sequence is expressed). Conversely, the inactive state of a promoter operably linked to a nucleic acid sequence refers to the state when the promoter is not actively regulating transcription of the nucleic acid sequence (i.e., the linked nucleic acid sequence is not expressed).

An inducible promoter may be induced by (or repressed by) one or more physiological condition(s), such as changes in light, pH, temperature, radiation, osmotic pressure, saline gradients, cell surface binding, and the concentration of one or more extrinsic or intrinsic inducing agent(s). An extrinsic inducer signal or inducing agent may comprise, without limitation, amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or combinations thereof.

Inducible promoters include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some embodiments, an inducible promoter is induced by engineered inducible proteins responding to plant hormone such as abscisic acid. For example, the dimerization domains of such engineered inducible proteins can be each fused with a DNA binding domain or transactivation domain to dimerize and activate a inducible promoter when abscisic acid is present (e.g., as described in Liang et al., Sci Signal. 2011 Mar. 15; 4(164):rs2, incorporated herein by reference).

In some embodiments, an inducer signal is an N-acyl homoserine lactone (AHL), which is a class of signaling molecules involved in bacterial quorum sensing. Quorum sensing is a method of communication between bacteria that enables the coordination of group based behavior based on population density. AHL can diffuse across cell membranes and is stable in growth media over a range of pH values. AHL can bind to transcriptional activators such as LuxR and stimulate transcription from cognate promoters.

In some embodiments, an inducer signal is anhydrotetracycline (aTc), which is a derivative of tetracycline that exhibits no antibiotic activity and is designed for use with tetracycline-controlled gene expression systems, for example, in bacteria.

In some embodiments, an inducer signal is isopropyl β-D-1-thiogalactopyranoside (IPTG), which is a molecular mimic of allolactose, a lactose metabolite that triggers transcription of the lac operon, and it is therefore used to induce protein expression where the gene is under the control of the lac operator. IPTG binds to the lac repressor and releases the tetrameric repressor from the lac operator in an allosteric manner, thereby allowing the transcription of genes in the lac operon, such as the gene coding for beta-galactosidase, a hydrolase enzyme that catalyzes the hydrolysis of β-galactosides into monosaccharides. The sulfur (S) atom creates a chemical bond which is non-hydrolyzable by the cell, preventing the cell from metabolizing or degrading the inducer. IPTG is an effective inducer of protein expression, for example, in the concentration range of 100 μM to 1.0 mM. The concentration used depends on the strength of induction required, as well as the genotype of cells or plasmid used. If lacIq, a mutant that over-produces the lac repressor, is present, then a higher concentration of IPTG may be necessary.

Other inducible promoter systems are known in the art and may be used in accordance with the present disclosure. Examples of inducible promoters include, without limitation, bacteriophage promoters (e.g. Pls1con, T3, T7, SP6, PL) and bacterial promoters (e.g., Pbad, PmgrB, Ptrc2, Plac/ara, Ptac, Pm), or hybrids thereof (e.g. PLlacO, PLtetO). Examples of bacterial promoters for use in accordance with the present disclosure include, without limitation, positively regulated E. coli promoters such as positively regulated 670 promoters (e.g., inducible pBad/araC promoter, Lux cassette right promoter, modified lamdba Prm promote, plac Or2-62 (positive), pBad/AraC with extra REN sites, pBad, P(Las) TetO, P(Las) CIO, P(Rh1), Pu, FecA, pRE, cadC, hns, pLas, pLux), GS promoters (e.g., Pdps), 632 promoters (e.g., heat shock) and σ54 promoters (e.g., glnAp2); negatively regulated E. coli promoters such as negatively regulated σ70 promoters (e.g., Promoter (PRM+), modified lamdba Prm promoter, TetR-TetR-4C P(Las) TetO, P(Las) CIO, P(Lac) IQ, RecA_DlexO_DLacO1, dapAp, FecA, Pspac-hy, pcI, plux-cI, plux-lac, CinR, CinL, glucose controlled, modified Pr, modified Prm+, FecA, Pcya, rec A (SOS), Rec A (SOS), EmrR_regulated, BetI_regulated, pLac_lux, pTet_Lac, pLac/Mnt, pTet/Mnt, LsrA/cI, pLux/cI, LacI, LacIQ, pLacIQ1, pLas/cI, pLas/Lux, pLux/Las, pRecA with LexA binding site, reverse BBa_R0011, pLacI/ara-1, pLacIq, rrnB P1, cadC, hns, PfhuA, pBad/araC, nhaA, OmpF, RcnR), σS promoters (e.g., Lutz-Bujard LacO with alternative sigma factor σ38), σ32 promoters (e.g., Lutz-Bujard LacO with alternative sigma factor σ32), and σ54 promoters (e.g., glnAp2); negatively regulated B. subtilis promoters such as repressible B. subtilis σA promoters (e.g., Gram-positive IPTG-inducible, Xyl, hyper-spank) and σB promoters. Other inducible microbial promoters may be used in accordance with the present disclosure.

In some embodiments, inducible promoters of the present disclosure function in eukaryotic cells (e.g., mammalian cells). Examples of inducible promoters for use eukaryotic cells include, without limitation, chemically-regulated promoters (e.g., alcohol-regulated promoters, tetracycline-regulated promoters, steroid-regulated promoters, metal-regulated promoters, and pathogenesis-related (PR) promoters) and physically-regulated promoters (e.g., temperature-regulated promoters and light-regulated promoters).

In some embodiments, the antibody produced by the antibody expression system described herein comprises a heavy chain and a light chain that are encoded on the same nucleic acid. As such, the antibody expression system comprises a promoter operably linked to a nucleic acid comprising a nucleotide sequence encoding one or more viral non-structural proteins, and comprising: (a) a first subgenomic viral promoter operably linked to a nucleotide sequence encoding an immunoglobulin heavy chain; and (b) a second subgenomic viral promoter operably linked to a nucleotide sequence encoding an immunoglobulin light chain. In some embodiments, part (a) is upstream of part (b). In some embodiments, part (b) is upstream of part (a). Being “upstream” means being on the 5′ side relative to another sequence/element in the same nucleic acid molecule.

In some embodiments, the nucleic acid further comprises additional regulatory sequences, including, without limitation, a 3′ untranslated region (3′UTR), and/or a poly-adenylation (polyA) signal sequence. In some embodiments, part (a) of the nucleic acid further comprises a nucleotide sequence encoding a 3′ untranslated region (3′UTR) downstream of the nucleotide sequence encoding the immunoglobulin heavy chain. In some embodiments, part (b) of the nucleic acid further comprises a nucleotide sequence encoding a 3′ untranslated region (3′UTR) downstream of the nucleotide sequence encoding the immunoglobulin light chain. In some embodiments, part (a) of the nucleic acid further comprises a nucleotide sequence encoding a 3′ untranslated region (3′UTR) downstream of the nucleotide sequence encoding the immunoglobulin heavy chain, and part (b) of the nucleic acid further comprises a nucleotide sequence encoding a 3′ untranslated region (3′UTR) downstream of the nucleotide sequence encoding the immunoglobulin light chain. A “3′ untranslated region (3′UTR)” refers to the section of mRNA that immediately follows the translation termination codon (stop codon). This region of the mRNA is not translated into proteins. Being “downstream” means being on the 3′ side relative to another sequence/element in the same nucleic acid molecule.

In some embodiments, the heavy chain and light chain are encoded on the same nucleic acid, and, when part (a) is upstream of part (b), part (b) further comprises a nucleotide sequence encoding a poly-adenylation signal sequence downstream of the 3′UTR. In some embodiments, the heavy chain and light chain are encoded on the same nucleic acid, and, when part (b) is upstream of part (a), part (a) further comprises a nucleotide sequence encoding a poly-adenylation signal sequence downstream of the 3′UTR. A “poly-adenylation signal sequence,” (also referred to as “polyA”) as used herein, refers to a sequence motif recognized by the RNA cleavage complex that cleaves the 3′-most part of a newly produced RNA and polyadenylates the end produced by this cleavage. The sequence of the polyadenylation signal varies between groups of eukaryotes. Most human polyadenylation sites contain the AAUAAA sequence.

In some embodiments, the polyA signal sequence comprises a transcriptional terminator. A “transcriptional terminator” is a nucleic acid sequence that causes transcription to stop. A terminator may be unidirectional or bidirectional. It is comprised of a DNA sequence involved in specific termination of an RNA transcript by an RNA polymerase. A terminator sequence prevents transcriptional activation of downstream nucleic acid sequences by upstream promoters. A terminator may be necessary in vivo to achieve desirable output expression levels (e.g., low output levels) or to avoid transcription of certain sequences.

The most commonly used type of terminator is a forward terminator. When placed downstream of a nucleic acid sequence that is usually transcribed, a forward transcriptional terminator will cause transcription to abort. In some embodiments, bidirectional transcriptional terminators are provided, which usually cause transcription to terminate on both the forward and reverse strand. In some embodiments, reverse transcriptional terminators are provided, which usually terminate transcription on the reverse strand only.

In prokaryotic systems, terminators usually fall into two categories (1) rho-independent terminators and (2) rho-dependent terminators. Rho-independent terminators are generally composed of palindromic sequence that forms a stem loop rich in G-C base pairs followed by several T bases. Without wishing to be bound by theory, the conventional model of transcriptional termination is that the stem loop causes RNA polymerase to pause, and transcription of the poly-A tail causes the RNA:DNA duplex to unwind and dissociate from RNA polymerase.

In eukaryotic systems, the terminator region may comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in some embodiments involving eukaryotes, a terminator may comprise a signal for the cleavage of the RNA. In some embodiments, the terminator signal promotes polyadenylation of the message. The terminator and/or polyadenylation site elements may serve to enhance output nucleic acid levels and/or to minimize read through between nucleic acids.

Terminators for use in accordance with the present disclosure include any terminator of transcription described herein or known to one of ordinary skill in the art. Examples of terminators include, without limitation, the termination sequences of genes such as, for example, the bovine growth hormone terminator, and viral termination sequences such as, for example, the SV40 terminator, spy, yejM, secG-leuU, thrLABC, rrnB T1, hisLGDCBHAFI, metZWV, rrnC, xapR, aspA and arcA terminator. In some embodiments, the termination signal may be a sequence that cannot be transcribed or translated, such as those resulting from a sequence truncation. In some embodiments, the transcriptional terminators is selected from BGH_TT, antigenomic-BGH_TT, rb_glob_TT, and antigenomic_HD-SV40_TT. The nucleotide sequences for non-limiting, exemplary transcriptional terminators are provided in Table 1.

TABLE 1
Non-limiting, exemplary transcriptional terminators

| Transcriptional | |
|---|---|
| Terminator | Nucleotide Sequence |
| BGH_polyA | CAGCCTCGACTGTGCCTTCTAGTTGCCAGCCATCTGTTGTTTGC |
| | CCCTCCCCCGTGCCTTCCTTGACCCTGGAAGGTGCCACTCCCAC |
| | TGTCCTTTCCTAATAAAATGAGGAAATTGCATCGCATTGTCTGA |
| | GTAGGTGTCATTCTATTCTGGGGGGTGGGGTGGGGCAGGACAG |
| | CAAGGGGGAGGATTGGGAAGAGAATAGCAGGCATGCTGGGGA |
| | TGCGGTGGGCTCTATGGC (SEQ ID NO: 1) |
| Rb_glob_polyA | TGAATTCACTCCTCAGGTGCAGGCTGCCTATCAGAAGGTGGTG |
| | GCTGGTGTGGCCAATGCCCTGGCTCACAAATACCACTGAGATC |
| | TTTTTCCCTCTGCCAAAAATTATGGGGACATCATGAAGCCCCTT |
| | GAGCATCTGACTTCTGGCTAATAAAGGAAATTTATTTTCATTGC |
| | AATAGTGTGTTGGAATTTTTTGTGTCTCTCACTCGGAAGGACAT |
| | ATGGGAGGGCAAATCATTTAAAACATCAGAATGAGTATTTGGT |
| | TTAGAGTTTGGCAACATATGCCCATATGCTGGCTGCCATGAAC |
| | AAAGGTTGGCTATAAAGAGGTCATCAGTATATGAAACAGCCCC |
| | CTGCTGTCCATTCCTTATTCCATAGAAAAGCCTTGACTTGAGGT |
| | TAGATTTTTTTTATATTTTGTTTTGTGTTATTTTTTTCTTTAACAT |
| | CCCTAAAATTTTCCTTACATGTTTTACTAGCCAGATTTTTCCTCC |
| | TCTCCTGACTACTCCCAGTCATAGCTGTCCCTCTTCTCTTATGG |
| | AGATCCCTCGACCTG (SEQ ID NO: 2) |
| SV40_polyA | AGCGGCCGCCTGCAGCTTAAGACCGGTAAGCTAAGCTACGCGT |
| | GCTAGCGGGCCCGTTAACTTGTTTATTGCAGCTTATAATGGTTA |
| | CAAATAAAGCAATAGCATCACAAATTTCACAAATAAAGCATTT |
| | TTTTCACTGCATTCTAGTTGTGGTTTGTCCAAACTCATCAATGT |
| | ATCTTATCATGTCTGGATCTTAATTAA (SEQ ID NO: 3) |

When the heavy chain and light chain are encoded on one nucleic acid, the single RNA replicon launched from the DNA molecule comprises a nucleotide sequence encoding one or more viral non-structural proteins, a first subgenomic viral promoter operably linked to a nucleotide sequence encoding an immunoglobulin heavy chain, and a second subgenomic viral promoter operably linked to a nucleotide sequence encoding an immunoglobulin light chain. In some embodiments, the single RNA replicon further comprises a 3′UTR downstream of the nucleotide sequence encoding the heavy chain. In some embodiments, the single RNA replicon further comprises a 3′UTR downstream of the nucleotide sequence encoding the light chain. In some embodiments, the single RNA replicon further comprises a 3′UTR downstream of the nucleotide sequence encoding the heavy chain, and further comprises a 3′UTR downstream of the nucleotide sequence encoding the light chain. In some embodiments, the single RNA replicon further comprises a polyA sequence at the 3′ end of the replicon, the addition of which is mediated by the polyA signal sequence.

In some embodiments, the antibody produced by the antibody expression system described herein comprises a heavy chain and a light chain encoded on two different nucleic acids. For example, in some embodiments, the antibody expression system comprises: (a) a promoter operably linked to a first nucleic acid comprising a nucleotide sequence encoding one or more viral non-structural proteins and a first subgenomic viral promoter operably linked to a nucleotide sequence encoding an immunoglobulin heavy chain; and (b) a promoter operably linked to a second nucleic acid comprising a nucleotide sequence encoding one or more viral non-structural proteins and a second subgenomic viral promoter operably linked to a nucleotide sequence encoding an immunoglobulin light chain.

In some embodiments, the first and/or second nucleic acids further comprises additional regulatory sequences, including, without limitation, a 3′ untranslated region (3′UTR), and/or a poly-adenylation (polyA) signal sequence. In some embodiments, the first nucleic acid further comprises a nucleotide sequence encoding a 3′ untranslated region (3′UTR) downstream of the nucleotide sequence encoding the immunoglobulin heavy chain. In some embodiments, the second nucleic acid further comprises a nucleotide sequence encoding a 3′ untranslated region (3′UTR) downstream of the nucleotide sequence encoding the immunoglobulin light chain. In some embodiments, the first nucleic acid further comprises a nucleotide sequence encoding a 3′ untranslated region (3′UTR) downstream of the nucleotide sequence encoding the immunoglobulin heavy chain, and the second nucleic acid further comprises a nucleotide sequence encoding a 3′ untranslated region (3′UTR) downstream of the nucleotide sequence encoding the immunoglobulin light chain.

In some embodiments, the first nucleic acid further comprises a nucleotide sequence encoding a poly-adenylation signal sequence downstream of the 3′UTR. In some embodiments, the second nucleic acid further comprises a nucleotide sequence encoding a poly-adenylation signal sequence downstream of the 3′UTR. In some embodiments, the first nucleic acid further comprises a nucleotide sequence encoding a poly-adenylation signal sequence downstream of the 3′UTR, and the second nucleic acid further comprises a nucleotide sequence encoding a poly-adenylation signal sequence downstream of the 3′UTR.

In some embodiments, the polyA signal sequence comprises a transcriptional terminator. Non-limiting, exemplary transcriptional terminators that may be used in accordance with the present disclosure include, BGH_TT, antigenomic-BGH_TT, rb_glob_TT, and antigenomic_HD-SV40_TT.

When the heavy chain and light chain are encoded on two different nucleic acids, the first RNA replicon launched from the antibody expression system comprises a nucleotide sequence encoding one or more viral non-structural proteins, and a first subgenomic viral promoter operably linked to a nucleotide sequence encoding an immunoglobulin heavy chain. The second RNA replicon launched from the antibody expression system comprises a nucleotide sequence encoding one or more viral non-structural proteins, and a second subgenomic viral promoter operably linked to a nucleotide sequence encoding an immunoglobulin light chain. In some embodiments, the first RNA replicons further comprises a 3′UTR downstream of the nucleotide sequence encoding the heavy chain. In some embodiments, the second RNA replicon further comprises a 3′UTR downstream of the nucleotide sequence encoding the light chain. In some embodiments, the first RNA replicons further comprises a 3′UTR downstream of the nucleotide sequence encoding the heavy chain, and the second RNA replicon further comprises a 3′UTR downstream of the nucleotide sequence encoding the light chain. In some embodiments, the first RNA replicon further comprises a polyA sequence at the 3′ end of the replicon, the addition of which is mediated by the polyA signal sequence. In some embodiments, the second RNA replicon further comprises a polyA sequence at the 3′ end of the replicon, the addition of which is mediated by the polyA signal sequence. In some embodiments, the first RNA replicon further comprises a polyA sequence at the 3′ end of the first replicon, and the second RNA replicon further comprises a polyA sequence at the 3′ end of the second replicon.

In some embodiments, regardless of whether the heavy chain and light chain of the antibody are encoded on one or two nucleic acids, the control the launch of the RNA replicon is an inducible promoter activated by a signal produced from a cell classifier. In some embodiments, the inducible promoter is repressed by a signal produced from a cell classifier (also referred to as a “cell state classifier”). A “cell classifier,” as used herein, refers to a system with multiple genetic circuits integrated together by transcriptional or translational control, which is able to sense a gene expression profile (e.g., microRNAs expression profile) in a cell and produce an output molecule accordingly. A non-limiting example is the cell classifier that senses the presence or absence of microRNAs as described in International Application No. PCT/US2017/044643 and International Application Publication No. WO 20116/040395, incorporated herein by reference. Other non-limiting examples of genetic circuits of systems that may be used in accordance with the present disclosure include, those described in Xie et al, Science, Vol. 333, Issue 6047, pp. 1307-1311, 2011; Miki et al., Cell Stem Cell, Vol. 16, issue 6, pp. 699-711, 2015; Sayeg et al., ACS Synth. Biol., 4 (7), pp 788-795, 2015; and in Ra et al., Front Cell Dev Biol. 2017; 5: 77, 2017).

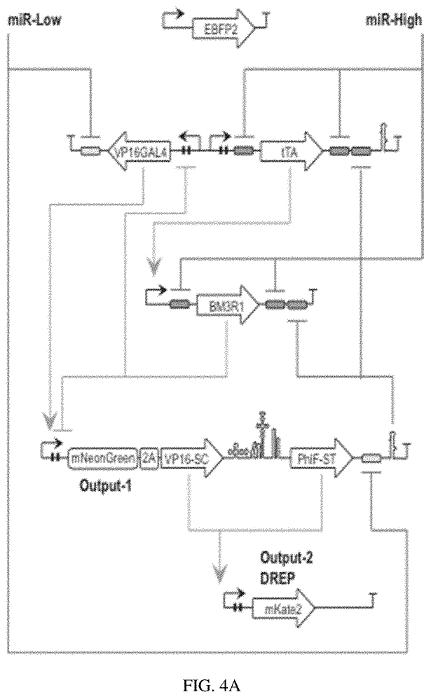

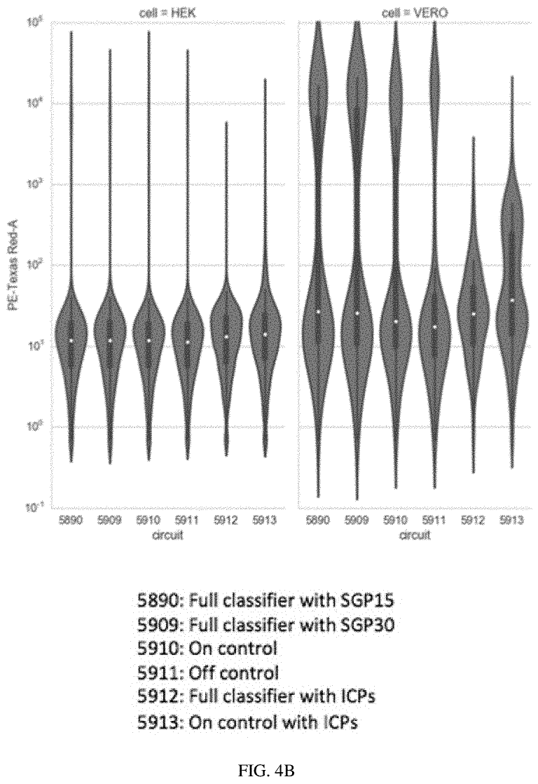

In some embodiments, the antibody expression system described herein is a component of a cell classifier, e.g., the output circuit (e.g., as shown in FIG. 4A herein). As such the antibody expression system may be regulated by the other components of the cell classifier, based on the gene expression profile detected in a cell and the antibody is produced as the output molecule once a specific gene profile is detected.

Once transcribed, the RNA replicon(s) are translated, the one or more viral non-structural proteins are translated. A “viral non-structural protein” is a protein encoded by a virus but that is not part of the viral particle. The viral non-structural proteins, in the context of DREP/VREP, functions to replicate the nucleotide sequences encoding the heavy chain and/or the light chain of the antibody from the RNA replicon via the sub-genomic viral promoters. Such replication driven by the viral sub-genomic promoter using the viral non-structural proteins enhances the replication level of the transgene (e.g., heavy chain and light chain of the antibody). In some embodiments, the viral non-structural proteins are from a single-strand positive-sense RNA viruses. In some embodiments, the viral non-structural proteins are from a Alphaviruse, belonging to the Togaviridae family. In some embodiments, the alphavirus is Sindbis or Venezuelan equine encephalitis virus. In some embodiments, the viral non-structural protein is an RNA-dependent RNA polymerase (RdRp) polyprotein P1234 (also termed NSP1-4 herein). Exemplary sequences for the viral non-structural proteins that can be used in accordance with the present disclosure are provided in Table 2 below.

TABLE 2
Viral non-structural proteins

| Viral non- | | | |
|---|---|---|---|
| structural | | | Amino Acid |
| protein | Virus | Nucleotide sequence | sequence |
| nsP1 | Venezuelan | ATGGAGAAAGTTCACGTTGACATCGAGGAAGACA | MEKVHVDIEED |
| | equine | GCCCATTCCTCAGAGCTTTGCAGCGGAGCTTCCCG | SPFLRALQRSFP |
| | encephalitis | CAGTTTGAGGTAGAAGCCAAGCAGGTCACTGATA | QFEVEAKQVTD |
| | virus | ATGACCATGCTAATGCCAGAGCGTTTTCGCATCTG | NDHANARAFSH |
| | | GCTTCAAAACTGATCGAAACGGAGGTGGACCCAT | LASKLIETEVDP |
| | | CCGACACGATCCTTGACATTGGAAGTGCGCCCGCC | SDTILDIGSAPA |
| | | CGCAGAATGTATTCTAAGCACAAGTATCATTGTAT | RRMYSKHKYH |
| | | CTGTCCGATGAGATGTGCGGAAGATCCGGACAGA | CICPMRCAEDP |
| | | TTGTATAAGTATGCAACTAAGCTGAAGAAAAACT | DRLYKYATKLK |
| | | GTAAGGAAATAACTGATAAGGAATTGGACAAGAA | KNCKEITDKEL |
| | | AATGAAGGAGCTCGCCGCCGTCATGAGCGACCCT | DKKMKELAAV |
| | | GACCTGGAAACTGAGACTATGTGCCTCCACGACG | MSDPDLETETM |
| | | ACGAGTCGTGTCGCTACGAAGGGCAAGTCGCTGTT | CLHDDESCRYE |
| | | TACCAGGATGTATACGCGGTTGACGGACCGACAA | GQVAVYQDVY |
| | | GTCTCTATCACCAAGCCAATAAGGGAGTTAGAGTC | AVDGPTSLYHQ |
| | | GCCTACTGGATAGGCTTTGACACCACCCCTTTTAT | ANKGVRVAYW |
| | | GTTTAAGAACTTGGCTGGAGCATATCCATCATACT | IGFDTTPFMFKN |
| | | CTACCAACTGGGCCGACGAAACCGTGTTAACGGC | LAGAYPSYSTN |
| | | TCGTAACATAGGCCTATGCAGCTCTGACGTTATGG | WADETVLTAR |
| | | AGCGGTCACGTAGAGGGATGTCCATTCTTAGAAA | NIGLCSSDVME |
| | | GAAGTATTTGAAACCATCCAACAATGTTCTATTCT | RSRRGMSILRK |
| | | CTGTTGGCTCGACCATCTACCACGAGAAGAGGGA | KYLKPSNNVLF |
| | | CTTACTGAGGAGCTGGCACCTGCCGTCTGTATTTC | SVGSTIYHEKR |
| | | ACTTACGTGGCAAGCAAAATTACACATGTCGGTGT | DLLRSWHLPSV |
| | | GAGACTATAGTTAGTTGCGACGGGTACGTCGTTAA | FHLRGKQNYTC |
| | | AAGAATAGCTATCAGTCCAGGCCTGTATGGGAAG | RCETIVSCDGY |
| | | CCTTCAGGCTATGCTGCTACGATGCACCGCGAGGG | VVKRIAISPGLY |
| | | ATTCTTGTGCTGCAAAGTGACAGACACATTGAACG | GKPSGYAATM |
| | | GGGAGAGGGTCTCTTTTCCCGTGTGCACGTATGTG | HREGFLCCKVT |
| | | CCAGCTACATTGTGTGACCAAATGACTGGCATACT | DTLNGERVSFP |
| | | GGCAACAGATGTCAGTGCGGACGACGCGCAAAAA | VCTYVPATLCD |
| | | CTGCTGGTTGGGCTCAACCAGCGTATAGTCGTCAA | QMTGILATDVS |
| | | CGGTCGCACCCAGAGAAACACCAATACCATGAAA | ADDAQKLLVGL |
| | | AATTACCTTTTGCCCGTAGTGGCCCAGGCATTTGC | NQRIVVNGRTQ |
| | | TAGGTGGGCAAAGGAATATAAGGAAGATCAAGAA | RNTNTMKNYLL |
| | | GATGAAAGGCCACTAGGACTACGAGATAGACAGT | PVVAQAFARW |
| | | TAGTCATGGGGTGTTGTTGGGCTTTTAGAAGGCAC | AKEYKEDQEDE |
| | | AAGATAACATCTATTTATAAGCGCCCGGATACCCA | RPLGLRDRQLV |
| | | AACCATCATCAAAGTGAACAGCGATTTCCACTCAT | MGCCWAFRRH |
| | | TCGTGCTGCCCAGGATAGGCAGTAACACATTGGA | KITSIYKRPDTQ |
| | | GATCGGGCTGAGAACAAGAATCAGGAAAATGTTA | TIIKVNSDFHSF |
| | | GAGGAGCACAAGGAGCCGTCACCTCTCATTACCG | VLPRIGSNTLEI |
| | | CCGAGGACGTACAAGAAGCTAAGTGCGCAGCCGA | GLRTRIRKMLE |
| | | TGAGGCTAAGGAGGTGCGTGAAGCCGAGGAGTTG | EHKEPSPLITAE |
| | | CGCGCAGCTCTACCACCTTTGGCAGCTGATGTTGA | DVQEAKCAAD |
| | | GGAGCCCACTCTGGAAGCCGATGTCGACTTGATGT | EAKEVREAEEL |
| | | TACAAGAGGCTGGGGCC (SEQ ID NO: 4) | RAALPPLAADV |
| | | | EEPTLEADVDL |
| | | | MLQEAGA (SEQ |
| | | | ID NO: 8) |
| nsP2 | Venezuelan | GGCTCAGTGGAGACACCTCGTGGCTTGATAAAGG | GSVETPRGLIKV |
| | equine | TTACCAGCTACGATGGCGAGGACAAGATCGGCTC | TSYDGEDKIGS |
| | encephalitis | TTACGCTGTGCTTTCTCCGCAGGCTGTACTCAAGA | YAVLSPQAVLK |
| | virus | GTGAAAAATTATCTTGCATCCACCCTCTCGCTGAA | SEKLSCIHPLAE |
| | | CAAGTCATAGTGATAACACACTCTGGCCGAAAAG | QVIVITHSGRKG |
| | | GGCGTTATGCCGTGGAACCATACCATGGTAAAGT | RYAVEPYHGKV |
| | | AGTGGTGCCAGAGGGACATGCAATACCCGTCCAG | VVPEGHAIPVQ |
| | | GACTTTCAAGCTCTGAGTGAAAGTGCCACCATTGT | DFQALSESATIV |
| | | GTACAACGAACGTGAGTTCGTAAACAGGTACCTG | YNEREFVNRYL |
| | | CACCATATTGCCACACATGGAGGAGCGCTGAACA | HHIATHGGALN |
| | | CTGATGAAGAATATTACAAAACTGTCAAGCCCAG | TDEEYYKTVKP |
| | | CGAGCACGACGGCGAATACCTGTACGACATCGAC | SEHDGEYLYDI |
| | | AGGAAACAGTGCGTCAAGAAAGAACTAGTCACTG | DRKQCVKKELV |
| | | GGCTAGGGCTCACAGGCGAGCTGGTGGATCCTCC | TGLGLTGELVD |
| | | CTTCCATGAATTCGCCTACGAGAGTCTGAGAACAC | PPFHEFAYESLR |
| | | GACCAGCCGCTCCTTACCAAGTACCAACCATAGG | TRPAAPYQVPTI |
| | | GGTGTATGGCGTGCCAGGATCAGGCAAGTCTGGC | GVYGVPGSGKS |
| | | ATCATTAAAAGCGCAGTCACCAAAAAAGATCTAG | GIIKSAVTKKDL |
| | | TGGTGAGCGCCAAGAAAGAAAACTGTGCAGAAAT | VVSAKKENCAE |
| | | TATAAGGGACGTCAAGAAAATGAAAGGGCTGGAC | IIRDVKKMKGL |
| | | GTCAATGCCAGAACTGTGGACTCAGTGCTCTTGAA | DVNARTVDSVL |
| | | TGGATGCAAACACCCCGTAGAGACCCTGTATATTG | LNGCKHPVETL |
| | | ACGAAGCTTTTGCTTGTCATGCAGGTACTCTCAGA | YIDEAFACHAG |
| | | GCGCTCATAGCCATTATAAGACCTAAAAAGGCAG | TLRALIAIIRPKK |
| | | TGCTCTGCGGGGATCCCAAACAGTGCGGTTTTTTT | AVLCGDPKQCG |
| | | AACATGATGTGCCTGAAAGTGCATTTTAACCACGA | FFNMMCLKVHF |
| | | GATTTGCACACAAGTCTTCCACAAAAGCATCTCTC | NHEICTQVFHK |
| | | GCCGTTGCACTAAATCTGTGACTTCGGTCGTCTCA | SISRRCTKSVTS |
| | | ACCTTGTTTTACGACAAAAAAATGAGAACGACGA | VVSTLFYDKKM |
| | | ATCCGAAAGAGACTAAGATTGTGATTGACACTAC | RTTNPKETKIVI |
| | | CGGCAGTACCAAACCTAAGCAGGACGATCTCATT | DTTGSTKPKQD |
| | | CTCACTTGTTTCAGAGGGTGGGTGAAGCAGTTGCA | DLILTCFRGWV |
| | | AATAGATTACAAAGGCAACGAAATAATGACGGCA | KQLQIDYKGNE |
| | | GCTGCCTCTCAAGGGCTGACCCGTAAAGGTGTGTA | IMTAAASQGLT |
| | | TGCCGTTCGGTACAAGGTGAATGAAAATCCTCTGT | RKGVYAVRYK |
| | | ACGCACCCACCTCAGAACATGTGAACGTCCTACTG | VNENPLYAPTS |
| | | ACCCGCACGGAGGACCGCATCGTGTGGAAAACAC | EHVNVLLTRTE |
| | | TAGCCGGCGACCCATGGATAAAAACACTGACTGC | DRIVWKTLAGD |
| | | CAAGTACCCTGGGAATTTCACTGCCACGATAGAG | PWIKTLTAKYP |
| | | GAGTGGCAAGCAGAGCATGATGCCATCATGAGGC | GNFTATIEEWQ |
| | | ACATCTTGGAGAGACCGGACCCTACCGACGTCTTC | AEHDAIMRHIL |
| | | CAGAATAAGGCAAACGTGTGTTGGGCCAAGGCTT | ERPDPTDVFQN |
| | | TAGTGCCGGTGCTGAAGACCGCTGGCATAGACAT | KANVCWAKAL |
| | | GACCACTGAACAATGGAACACTGTGGATTATTTTG | VPVLKTAGIDM |
| | | AAACGGACAAAGCTCACTCAGCAGAGATAGTATT | TTEQWNTVDYF |
| | | GAACCAACTATGCGTGAGGTTCTTTGGACTCGATC | ETDKAHSAEIV |
| | | TGGACTCCGGTCTATTTTCTGCACCCACTGTTCCGT | LNQLCVRFFGL |
| | | TATCCATTAGGAATAATCACTGGGATAACTCCCCG | DLDSGLFSAPT |
| | | TCGCCTAACATGTACGGGCTGAATAAAGAAGTGG | VPLSIRNNHWD |
| | | TCCGTCAGCTCTCTCGCAGGTACCCACAACTGCCT | NSPSPNMYGLN |
| | | CGGGCAGTTGCCACTGGAAGAGTCTATGACATGA | KEVVRQLSRRY |
| | | ACACTGGTACACTGCGCAATTATGATCCGCGCATA | PQLPRAVATGR |
| | | AACCTAGTACCTGTAAACAGAAGACTGCCTCATGC | VYDMNTGTLR |
| | | TTTAGTCCTCCACCATAATGAACACCCACAGAGTG | NYDPRINLVPV |
| | | ACTTTTCTTCATTCGTCAGCAAATTGAAGGGCAGA | NRRLPHALVLH |
| | | ACTGTCCTGGTGGTCGGGGAAAAGTTGTCCGTCCC | HNEHPQSDFSSF |
| | | AGGCAAAATGGTTGACTGGTTGTCAGACCGGCCT | VSKLKGRTVLV |
| | | GAGGCTACCTTCAGAGCTCGGCTGGATTTAGGCAT | VGEKLSVPGKM |
| | | CCCAGGTGATGTGCCCAAATATGACATAATATTTG | VDWLSDRPEAT |
| | | TTAATGTGAGGACCCCATATAAATACCATCACTAT | FRARLDLGIPGD |
| | | CAGCAGTGTGAAGACCATGCCATTAAGCTTAGCAT | VPKYDIIFVNVR |
| | | GTTGACCAAGAAAGCTTGTCTGCATCTGAATCCCG | TPYKYHHYQQC |
| | | GCGGAACCTGTGTCAGCATAGGTTATGGTTACGCT | EDHAIKLSMLT |
| | | GACAGGGCCAGCGAAAGCATCATTGGTGCTATAG | KKACLHLNPGG |
| | | CGCGGCTGTTCAAGTTTTCCCGGGTATGCAAACCG | TCVSIGYGYAD |
| | | AAATCCTCACTTGAAGAGACGGAAGTTCTGTTTGT | RASESIIGAIARL |
| | | ATTCATTGGGTACGATCGCAAGGCCCGTACGCACA | FKFSRVCKPKSS |
| | | ATCCTTACAAGCTTTCATCAACCTTGACCAACATT | LEETEVLFVFIG |
| | | TATACAGGTTCCAGACTCCACGAAGCCGGATGT | YDRKARTHNPY |
| | | (SEQ ID NO: 5) | KLSSTLTNIYTG |
| | | | SRLHEAGC |
| | | | (SEQ ID NO: 9) |
| nsP3 | Venezuelan | GCACCCTCATATCATGTGGTGCGAGGGGATATTGC | APSYHVVRGDI |
| | equine | CACGGCCACCGAAGGAGTGATTATAAATGCTGCT | ATATEGVIINAA |
| | encephalitis | AACAGCAAAGGACAACCTGGCGGAGGGGTGTGCG | NSKGQPGGGVC |
| | virus | GAGCGCTGTATAAGAAATTCCCGGAAAGCTTCGA | GALYKKFPESF |
| | | TTTACAGCCGATCGAAGTAGGAAAAGCGCGACTG | DLQPIEVGKAR |
| | | GTCAAAGGTGCAGCTAAACATATCATTCATGCCGT | LVKGAAKHIIH |
| | | AGGACCAAACTTCAACAAAGTTTCGGAGGTTGAA | AVGPNFNKVSE |
| | | GGTGACAAACAGTTGGCAGAGGCTTATGAGTCCA | VEGDKQLAEAY |
| | | TCGCTAAGATTGTCAACGATAACAATTACAAGTCA | ESIAKIVNDNNY |
| | | GTAGCGATTCCACTGTTGTCCACCGGCATCTTTTC | KSVAIPLLSTGIF |
| | | CGGGAACAAAGATCGACTAACCCAATCATTGAAC | SGNKDRLTQSL |
| | | CATTTGCTGACAGCTTTAGACACCACTGATGCAGA | NHLLTALDTTD |
| | | TGTAGCCATATACTGCAGGGACAAGAAATGGGAA | ADVAIYCRDKK |
| | | ATGACTCTCAAGGAAGCAGTGGCTAGGAGAGAAG | WEMTLKEAVA |
| | | CAGTGGAGGAGATATGCATATCCGACGACTCTTCA | RREAVEEICISD |
| | | GTGACAGAACCTGATGCAGAGCTGGTGAGGGTGC | DSSVTEPDAEL |
| | | ATCCGAAGAGTTCTTTGGCTGGAAGGAAGGGCTA | VRVHPKSSLAG |
| | | CAGCACAAGCGATGGCAAAACTTTCTCATATTTGG | RKGYSTSDGKT |
| | | AAGGGACCAAGTTTCACCAGGCGGCCAAGGATAT | FSYLEGTKFHQ |
| | | AGCAGAAATTAATGCCATGTGGCCCGTTGCAACG | AAKDIAEINAM |
| | | GAGGCCAATGAGCAGGTATGCATGTATATCCTCG | WPVATEANEQ |
| | | GAGAAAGCATGAGCAGTATTAGGTCGAAATGCCC | VCMYILGESMS |
| | | CGTCGAAGAGTCGGAAGCCTCCACACCACCTAGC | SIRSKCPVEESE |
| | | ACGCTGCCTTGCTTGTGCATCCATGCCATGACTCC | ASTPPSTLPCLCI |
| | | AGAAAGAGTACAGCGCCTAAAAGCCTCACGTCCA | HAMTPERVQRL |
| | | GAACAAATTACTGTGTGCTCATCCTTTCCATTGCC | KASRPEQITVCS |
| | | GAAGTATAGAATCACTGGTGTGCAGAAGATCCAA | SFPLPKYRITGV |
| | | TGCTCCCAGCCTATATTGTTCTCACCGAAAGTGCC | QKIQCSQPILFSP |
| | | TGCGTATATTCATCCAAGGAAGTATCTCGTGGAAA | KVPAYIHPRKY |
| | | CACCACCGGTAGACGAGACTCCGGAGCCATCGGC | LVETPPVDETPE |
| | | AGAGAACCAATCCACAGAGGGGACACCTGAACAA | PSAENQSTEGTP |
| | | CCACCACTTATAACCGAGGATGAGACCAGGACTA | EQPPLITEDETR |
| | | GAACGCCTGAGCCGATCATCATCGAAGAGGAAGA | TRTPEPIIIEEEE |
| | | AGAGGATAGCATAAGTTTGCTGTCAGATGGCCCG | EDSISLLSDGPT |
| | | ACCCACCAGGTGCTGCAAGTCGAGGCAGACATTC | HQVLQVEADIH |
| | | ACGGGCCGCCCTCTGTATCTAGCTCATCCTGGTCC | GPPSVSSSSWSI |
| | | ATTCCTCATGCATCCGACTTTGATGTGGACAGTTT | PHASDFDVDSL |
| | | ATCCATACTTGACACCCTGGAGGGAGCTAGCGTG | SILDTLEGASVT |
| | | ACCAGCGGGGCAACGTCAGCCGAGACTAACTCTT | SGATSAETNSY |
| | | ACTTCGCAAAGAGTATGGAGTTTCTGGCGCGACCG | FAKSMEFLARP |
| | | GTGCCTGCGCCTCGAACAGTATTCAGGAACCCTCC | VPAPRTVFRNPP |
| | | ACATCCCGCTCCGCGCACAAGAACACCGTCACTTG | HPAPRTRTPSLA |
| | | CACCCAGCAGGGCCTGCTCGAGAACCAGCCTAGT | PSRACSRTSLVS |
| | | TTCCACCCCGCCAGGCGTGAATAGGGTGATCACTA | TPPGVNRVITRE |
| | | GAGAGGAGCTCGAGGCGCTTACCCCGTCACGCAC | ELEALTPSRTPS |
| | | TCCTAGCAGGTCGGTCTCGAGAACCAGCCTGGTCT | RSVSRTSLVSNP |
| | | CCAACCCGCCAGGCGTAAATAGGGTGATTACAAG | PGVNRVITREEF |
| | | AGAGGAGTTTGAGGCGTTCGTAGCACAACAACAA | EAFVAQQQ |
| | | (SEQ ID NO: 6) | (SEQ ID NO: 10) |
| nsP4 | Venezuelan | TACATCTTTTCCTCCGACACCGGTCAAGGGCATTT | YIFSSDTGQGHL |
| | equine | ACAACAAAAATCAGTAAGGCAAACGGTGCTATCC | QQKSVRQTVLS |
| | encephalitis | GAAGTGGTGTTGGAGAGGACCGAATTGGAGATTT | EVVLERTELEIS |
| | virus | CGTATGCCCCGCGCCTCGACCAAGAAAAAGAAGA | YAPRLDQEKEE |
| | | ATTACTACGCAAGAAATTACAGTTAAATCCCACAC | LLRKKLQLNPT |
| | | CTGCTAACAGAAGCAGATACCAGTCCAGGAAGGT | PANRSRYQSRR |
| | | GGAGAACATGAAAGCCATAACAGCTAGACGTATT | VENMKAITARR |
| | | CTGCAAGGCCTAGGGCATTATTTGAAGGCAGAAG | ILQGLGHYLKA |
| | | GAAAAGTGGAGTGCTACCGAACCCTGCATCCTGTT | EGKVECYRTLH |
| | | CCTTTGTATTCATCTAGTGTGAACCGTGCCTTTTCA | PVPLYSSSVNR |
| | | AGCCCCAAGGTCGCAGTGGAAGCCTGTAACGCCA | AFSSPKVAVEA |
| | | TGTTGAAAGAGAACTTTCCGACTGTGGCTTCTTAC | CNAMLKENFPT |
| | | TGTATTATTCCAGAGTACGATGCCTATTTGGACAT | VASYCIIPEYDA |
| | | GGTTGACGGAGCTTCATGCTGCTTAGACACTGCCA | YLDMVDGASC |
| | | GTTTTTGCCCTGCAAAGCTGCGCAGCTTTCCAAAG | CLDTASFCPAK |
| | | AAACACTCCTATTTGGAACCCACAATACGATCGGC | LRSFPKKHSYLE |
| | | AGTGCCTTCAGCGATCCAGAACACGCTCCAGAAC | PTIRSAVPSAIQ |
| | | GTCCTGGCAGCTGCCACAAAAAGAAATTGCAATG | NTLQNVLAAAT |
| | | TCACGCAAATGAGAGAATTGCCCGTATTGGATTCG | KRNCNVTQMR |
| | | GCGGCCTTTAATGTGGAATGCTTCAAGAAATATGC | ELPVLDSAAFN |
| | | GTGTAATAATGAATATTGGGAAACGTTTAAAGAA | VECFKKYACNN |
| | | AACCCCATCAGGCTTACTGAAGAAAACGTGGTAA | EYWETFKENPI |
| | | ATTACATTACCAAATTAAAAGGACCAAAAGCTGC | RLTEENVVNYI |
| | | TGCTCTTTTTGCGAAGACACATAATTTGAATATGT | TKLKGPKAAAL |
| | | TGCAGGACATACCAATGGACAGGTTTGTAATGGA | FAKTHNLNMLQ |
| | | CTTAAAGAGAGACGTGAAAGTGACTCCAGGAACA | DIPMDRFVMDL |
| | | AAACATACTGAAGAACGGCCCAAGGTACAGGTGA | KRDVKVTPGTK |
| | | TCCAGGCTGCCGATCCGCTAGCAACAGCGTATCTG | HTEERPKVQVI |
| | | TGCGGAATCCACCGAGAGCTGGTTAGGAGATTAA | QAADPLATADL |
| | | ATGCGGTCCTGCTTCCGAACATTCATACACTGTTT | CGIHRELVRRL |
| | | GATATGTCGGCTGAAGACTTTGACGCTATTATAGC | NAVLLPNIHTLF |
| | | CGAGCACTTCCAGCCTGGGGATTGTGTTCTGGAAA | DMSAEDFDAIIA |
| | | CTGACATCGCGTCGTTTGATAAAAGTGAGGACGA | EHFQPGDCVLE |
| | | CGCCATGGCTCTGACCGCGTTAATGATTCTGGAAG | TDIASFDKSEDD |
| | | ACTTAGGTGTGGACGCAGAGCTGTTGACGCTGATT | AMALTALMILE |
| | | GAGGCGGCTTTCGGCGAAATTTCATCAATACATTT | DLGVDAELLTLI |
| | | GCCCACTAAAACTAAATTTAAATTCGGAGCCATGA | EAAFGEISSIHLP |
| | | TGAAATCTGGAATGTTCCTCACACTGTTTGTGAAC | TKTKFKFGAM |
| | | ACAGTCATTAACATTGTAATCGCAAGCAGAGTGTT | MKSGMFLTLFV |
| | | GAGAGAACGGCTAACCGGATCACCATGTGCAGCA | NTVINIVIASRV |
| | | TTCATTGGAGATGACAATATCGTGAAAGGAGTCA | LRERLTGSPCA |
| | | AATCGGACAAATTAATGGCAGACAGGTGCGCCAC | AFIGDDNIVKG |
| | | CTGGTTGAATATGGAAGTCAAGATTATAGATGCTG | VKSDKLMADR |
| | | TGGTGGGCGAGAAAGCGCCTTATTTCTGTGGAGG | CATWLNMEVKI |
| | | GTTTATTTTGTGTGACTCCGTGACCGGCACAGCGT | IDAVVGEKAPY |
| | | GCCGTGTGGCAGACCCCCTAAAAAGGCTGTTTAA | FCGGFILCDSVT |
| | | GCTTGGCAAACCTCTGGCAGCAGACGATGAACAT | GTACRVADPLK |
| | | GATGATGACAGGAGAAGGGCATTGCATGAAGAGT | RLFKLGKPLAV |
| | | CAACACGCTGGAACCGAGTGGGTATTCTTTCAGAG | DDEHDDDRRR |
| | | CTGTGCAAGGCAGTAGAATCAAGGTATGAAACCG | ALHEESTRWNR |
| | | TAGGAACTTCCATCATAGTTATGGCCATGACTACT | VGILPELCKAVE |
| | | CTAGCTAGCAGTGTTAAATCATTCAGCTACCTGAG | SRYETVGTSIIV |
| | | AGGGGCCCCTATAACTCTCTACGGC (SEQ ID | MAMTTLASSVK |
| | | NO: 7) | SFSYLRGAPITL |
| | | | YG (SEQ ID NO: |
| | | | 11) |

Upon translation, P1234 is rapidly cleaved into P123 and nsP4 by autoproteolytic activity originating from the nsP2 (proteinase) portion of the polyprotein. Alphaviral RNA synthesis occurs at the plasma membrane of a cell, where the nsPs, together with alphaviral RNA, form membrane invaginations (or “spherules”). These spherules contain dsRNA created by replication of “+” strand viral genomic RNA into “−” strand anti-genomic RNA. The “−” strand serves as a template from which additional “+” strand genomic RNA (synthesized from the 5′UTR) or a shorter subsequence of the genomic RNA (termed subgenomic RNA) is synthesized from the subgenomic viral promoter region located near the end of the nonstructural protein ORF. The “+” strand genomic RNA and the subgenomic RNA are exported out of the spherules into the cytoplasm where they are translated by endogenous ribosomes. The exported “+” strand genomic RNA can associate with nsPs and form additional spherules, thus resulting in exponential increase of replicon RNA.

The viral non-structural proteins facilitate the replication of the nucleotide sequences encoding the heavy chain and/or light chain via the subgenomic viral promoters (also referred to as “subgenomic promoters” herein). A “subgenomic viral promoter” refers to a promoter the drives the transcription of subgenomic mRNAs. Typically, an mRNA is transcribed from genomic DNAs and episomal DNAs (e.g., plasmids). Some viruses has the ability to transcribe subgenomic mRNAs from a RNA replicon that is produced from its genomic DNA. Many positive-sense RNA viruses produce subgenomic mRNAs as one of the common infection techniques used by these viruses and generally transcribe late viral genes. Subgenomic viral promoters range from 20 nucleotide (Sindbis virus) to over 100 nucleotides (Beet necrotic yellow vein virus) and are usually found upstream of the transcription start. In some embodiments, the subgenomic viral promoter is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nucleotides long, or longer. Subgenomic viral promoters have been described in the art, e.g., in PCT Application Publication No. WO 2016/040359, and Wagner et al., Nature Chemical Biology, DIO: 10.1038/s41589-018-0146-9 (2018), incorporated herein by reference. Non-limiting, Exemplary subgenomic viral promoters and their sequences are provided in Table 3.

TABLE 3
Non-limiting, Exemplary Subgenomic
viral promoters

| Subgenomic | |
|---|---|
| viral | |
| promoter | Nucleotide Sequence |
| SGP30 | ATGGACTACGACATAGTCTAGTCCGCCAAG |
| | (SEQ ID NO: 12) |
| SGP15 | ATGGACTACGACATA (SEQ ID NO: 13) |
| SGP5 | ATGGA |

In some embodiments, the transgene that is upstream is expressed at a lower level relative to the transgene that is downstream. In some embodiments, the first subgenomic viral promoter and the second subgenomic viral promoter are the same. In some embodiments, the first subgenomic viral promoter and the second subgenomic viral promoter are different. Different subgenomic viral promoters may lead to different expression levels of the antibody heavy chain and light chain. In some embodiments, the first subgenomic viral promoter is SGP5, SGP15, or SGP 30 (see Table 3 above). In some embodiments, the second subgenomic viral promoter is SGP5, SGP15, or SGP 30 (see Table 3 above).

It was demonstrated herein that the expression level of the complete antibody can be regulated by adjusting the relative expression level of the heavy chain and the light chain. In some embodiments, different combinations of the first subgenomic promoter and the second subgenomic promoter are used to achieve different relative expression level of the heavy chain and the light chain. For example, in some embodiments, the first subgenomic promoter is SGP 5 and the second subgenomic promoter is SGP5. In some embodiments, the first subgenomic promoter is SGP30 and the second subgenomic promoter is SGP5. In some embodiments, the first subgenomic promoter is SGP15 and the second subgenomic promoter is SGP5. In some embodiments, the first subgenomic promoter is SGP5 and the second subgenomic promoter is SGP30. In some embodiments, the first subgenomic promoter is SGP30 and the second subgenomic promoter is SGP30. In some embodiments, the first subgenomic promoter is SGP5 and the second subgenomic promoter is SGP15. In some embodiments, the first subgenomic viral promoter is SGP30 and the second subgenomic viral promoter is SGP15. In some embodiments, the first subgenomic promoter is SGP15 and the second subgenomic promoter is SGP15.

In some embodiments, 3′UTRs and/or polyA signals can be added downstream of the nucleotide sequence encoding the heavy chain and/or light chain to further regulate the relative expression level of the heavy chain and the light chain. In some embodiments, when the heavy chain and light chain are encoded on the same nucleotide, a 3′ UTR is added to the nucleotide sequence encoding the heavy chain, and a 3′UTR as well as a polyA signal is added downstream of the nucleotide sequence encoding the light chain.

In some embodiments, a ribozyme can be added between the 3′UTR and the poly A signal sequence. A “ribozyme” is an RNA molecule that is capable of catalyzing specific biochemical reactions, similar to the action of protein enzymes. Suitable ribozymes that may be used in accordance with the present disclosure and their respective sequences include, without limitation: RNase P, hammerhead ribozymes, Hepatitis delta virus ribozymes, hairpin ribozymes, twister ribozymes, twister sister ribozymes, pistol ribozymes, hatchet ribozymes, glmS ribozymes, varkud satellite ribozymes, and spliceozyme. Naturally occurring ribozymes may be used. Further, ribozymes engineered such that they cleave their substrates in cis or in trans, e.g., as described in Carbonell et al. Nucleic Acids Res. 2011 March; 39(6): 2432-2444, may be used. Minimal ribozymes (i.e., the minimal sequence a ribozyme needs for its function, e.g., as described in Scott et al., Prog Mol Biol Transl Sci. 2013; 120: 1-23) may also be used in accordance with the present disclosure.

In some embodiments, the light chain and heavy chain are expressed at a molar ratio of 1:1 to 5:1. For example, the light chain and heavy chain may be expressed at a molar ratio of (light chain:heavy chain) 1:1 to 5:1, 1:1 to 4:1, 1:1 to 3:1, 1:1 to 2:1, 2:1 to 5:1, 2:1 to 4:1, 2:1 to 3:1, 3:1 to 5:1, 3:1 to 4:1, or 4:1 to 5:1. In some embodiments, the light chain and heavy chain are expressed at a molar ratio of 1:1, 2:1, 3:1, 4:1, or 5:1. In some embodiments, the light chain and heavy chain are expressed at a molar ratio of 3:1. In some embodiments, the light chain and heavy chain are expressed at a molar ratio of 2.5:1. In some embodiments, the light chain and heavy chain are expressed at a molar ratio of 2.47:1 (this ratio achieved the most efficient production of a complete antibody, as shown in FIG. 21).

In some embodiments, the nucleic acid(s) in the antibody expression system described herein comprises further sequence elements for further regulation of the antibody expression. In some embodiments, the nucleic acid(s) in the antibody expression system further comprises nucleotide sequences encoding one or more (e.g., 1, 2, 3, 4, 5 or more) cleavage sites for an endoribonuclease. As such, the RNA replicon launched from the nucleic acids in the antibody expression system comprises one or more (e.g., 1, 2, 3, 4, 5 or more) cleavage sites for an endoribonuclease and can be cleaved by respective endoribonucleases. The cleavage sites may be located anywhere in the RNA replicon except for the sequences encoding the heavy chain and the light chain, e.g., the 3′UTRs. If more than one cleavage sites are present, they may be at the same or different locations in the RNA replicon. The presence of the cleavage sites for endoribonucleases allows the destruction of the RNA replicon by these endoribonucleases, thus eliminating the expression of the antibody when desired.

In some embodiments, the antibody expression system further comprises a promoter operably linked to a nucleotide sequence encoding an endoribonuclease. In some embodiments, the nucleotide sequence encoding the endoribonuclease and the promoter it is operably linked to are located on the same nucleic acid encoding the heavy chain and/or the light chain. In some embodiments, the nucleotide sequence encoding the endoribonuclease and the promoter it is operably linked to are located on a separate nucleic acid. In some embodiments, the promoter operably linked the endoribonuclease is an inducible promoter. Any inducible promoters described herein or known in the art may be used. In some embodiments, the inducible promoter is regulated by a small molecule. In some embodiments, the small molecule is doxycycline or abscisic acid.

An “endoribonuclease,” as used herein, refers to a nuclease that cleaves an RNA molecule in a sequence specific manner, e.g., at a cleavage site. Sequence-specific endoribonucleases have been described in the art. For example, the Pyrococcus furiosus CRISPR-associated endoribonuclease 6 (Cas6) is found to cleave RNA molecules in a sequence-specific manner (Carte et al., Genes & Dev. 2008. 22: 3489-3496, incorporated herein by reference). In another example, Csy4, a CRISPR-associated endoribonuclease found in Pseudomonas aeruginosa. The Csy4 protein recognizes a 28-nucleotide RNA repeat and cleaves between nucleotides 20 and 21. In some embodiments, endoribonucleases that cleave RNA molecules in a sequence-specific manner are engineered, which recognize an 8-nucleotide (nt) RNA sequence and make a single cleavage in the target (Choudhury et al., Nature Communications 3, 1147 (2012), incorporated herein by reference). In some embodiments, the endoribonuclease belongs to the CRISPR-associated endoribonuclease 6 (Cas6) family. Cas6 nucleases from different bacterial species may be used. Non-limiting examples of Cas6 family nucleases include Csy4, Cse3, Cas6, Csy 13, CasE, and variants thereof.

A “recognition site for an endoribonuclease” refers to a ribonucleotide sequence that is recognized, bound, and cleaved by the endoribonuclease. The recognition site for an endoribonuclease may be 4-20 nucleotides long. For example, the recognition site may be 4, 5, 6, 7, 8, 9, 10, 11, 12, 13, 14, 15, 16, 17, 18, 19, or 20 nucleotides long. In some embodiments, endoribonuclease recognition sites that are shorter than 4 ribonucleotides or longer than 20 nucleotides are used. Table 4 provides the amino acid and nucleotide sequences of exemplary endoribonucleases and their respective recognition sites.

TABLE 4
Non-limiting, Exemplary Endoribonucleases
and Cleavage Sites

| Endoribonuclease/ | | | Cleavage site |
|---|---|---|---|
| Bacterial species | Amino acid sequence | Gene Sequence | sequence |
| Cas6/Pyrococcus/ | MRFLIRLVPEDKDR | ATGCGCTTCCTCATTCGTCTCGTGCCT | GTTACAAT |
| furiosus | AFKVPYNHQYYLQ | GAGGATAAGGATCGGGCCTTTAAAGT | AAGACTAA |
| | GLIYNAIKSSNPKL | GCCATATAACCATCAGTATTACCTGC | ATAGAATT |
| | ATYLHEVKGPKLFT | AGGGCCTCATCTATAATGCCATCAAA | GAAAG |
| | YSLFMAEKREHPK | TCCTCCAATCCGAAGCTGGCCACCTA | (SEQ ID NO: |
| | GLPYFLGYKKGFFY | CCTGCATGAGGTGAAGGGTCCCAAAC | 22) |
| | FSTCVPEIAEALVN | TGTTCACCTACAGCCTGTTTATGGCCG | |
| | GLLMNPEVRLWDE | AAAAACGCGAACACCCTAAGGGGCTG | |
| | RFYLHEIKVLREPK | CCTTACTTTTTGGGGTACAAGAAGGG | |
| | KFNGSTFVTLSPIA | CTTCTTCTACTTTTCTACCTGCGTGCC | |
| | VTVVRKGKSYDVP | GGAGATCGCTGAAGCACTGGTCAACG | |
| | PMEKEFYSIIKDDL | GACTCCTGATGAATCCAGAGGTGCGC | |
| | QDKYVMAYGDKPP | CTGTGGGACGAACGCTTCTACCTGCA | |
| | SEFEMEVLIAKPKR | CGAAATTAAGGTTTTGAGAGAGCCTA | |
| | FRIKPGIYQTAWHL | AGAAGTTCAACGGCTCTACCTTCGTC | |
| | VFRAYGNDDLLKV | ACCCTGTCTCCGATTGCTGTGACTGTC | |
| | GYEVGFGEKNSLG | GTGAGGAAGGGTAAGAGTTATGATGT | |
| | FGMVKVEGNKTTK | CCCCCCTATGGAGAAGGAGTTTTACA | |
| | EAEEQEKITFNSRE | GTATCATCAAAGACGACCTGCAAGAT | |
| | ELKTGV (SEQ ID | AAGTATGTGATGGCCTACGGCGACAA | |
| | NO: 14) | GCCCCCATCCGAATTCGAGATGGAGG | |
| | | TGCTGATTGCTAAGCCGAAACGGTTT | |
| | | CGTATTAAGCCTGGCATCTATCAGAC | |
| | | AGCCTGGCACCTGGTTTTTAGGGCCTA | |
| | | CGGAAACGACGACCTGCTGAAGGTTG | |
| | | GTTACGAGGTTGGGTTCGGAGAAAAG | |
| | | AACTCCCTGGGATTCGGCATGGTGAA | |
| | | GGTGGAGGGGAACAAGACCACAAAA | |
| | | GAAGCTGAAGAGCAGGAAAAGATCA | |
| | | CCTTCAACTCTCGCGAGGAGCTGAAG | |
| | | ACCGGCGTGTGA (SEQ ID NO: 18) | |
| Csy4/ | MDHYLDIRLRPDPE | ATGGACCACTATCTGGACATCAGACT | GTTCACTG |
| Pseudomonas | FPPAQLMSVLDSKL | GAGGCCCGATCCTGAGTTCCCTCCCG | CCGTATAG |
| aeruginosa | HQALVAQGGDRIG | CCCAGCTGATGAGCGTGCTGTTTGGC | GCAGCTAA |
| | VSFPDLDESRSRLG | AAGCTGCATCAGGCTCTGGTCGCCCA | GAAA (SEQ |
| | ERLRIHASADDLRA | AGGCGGAGACAGAATCGGCGTGTCCT | ID NO: 23) |
| | LLARPWLEGLRDH | TCCCCGACCTGGACGAGTCCCGGAGT | |
| | LQFGEPAVVPHPTP | CGCCTGGGCGAGCGGCTGAGAATCCA | |
| | YRQVSRVQAKSNP | CGCCAGCGCAGACGATCTGCGCGCCC | |
| | ERLRRRLMRRHDL | TGCTGGCCCGGCCTTGGCTGGAGGGC | |
| | SEEEARKRIPDTVA | CTGCGGGATCATCTGCAGTTTGGCGA | |
| | RALDLPFVTLRSQS | GCCCGCCGTGGTGCCACACCCAACAC | |
| | TGQHFRLFIRHGPL | CCTACCGCCAGGTGAGCCGCGTGCAG | |
| | QVTAEEGGFTCYG | GCCAAGTCAAATCCCGAGAGACTGCG | |
| | LSKGGFVPWF (SEQ | GCGGAGGCTGATGAGGCGACATGATC | |
| | ID NO: 15) | TGAGCGAGGAGGAGGCCAGAAAGAG | |
| | | AATCCCCGACACAGTGGCCAGAGCCC | |
| | | TGGATCTGCCATTTGTGACCCTGCGGA | |
| | | GCCAGAGCACTGGCCAGCATTTCAGA | |
| | | CTGTTCATCAGACACGGGCCCCTGCA | |
| | | GGTGACAGCCGAGGAGGGCGGATTTA | |
| | | CATGCTATGGCCTGTCTAAAGGCGGC | |
| | | TTCGTGCCCTGGTTCTGA(SEQIDNO: | |
| | | 19) | |
| CasE/Escherichia | MYLSKIIIARAWSR | ATGTACCTCAGTAAGATCATCATCGC | GAGTTCCC |
| coli | DLYQLHQELWHLF | CCGCGCTTGGTCCCGTGACCTGTACCA | CGCGCCAG |
| | PNRPDAARDFLFHV | ACTGCACCAAGAGCTCTGGCACCTCT | CGGGGATA |
| | EKRNTPEGCHVLL | TCCCCAACAGGCCAGATGCCGCTAGA | AACCG |
| | QSAQMPVSTAVAT | GACTTCCTGTTCCACGTGGAGAAGCG | (SEQ ID NO: |
| | VIKTKQVEFQLQVG | TAACACCCCCGAAGGGTGCCACGTGC | 24) |
| | VPLYFRLRANPIKTI | TGTTGCAGAGTGCCCAGATGCCAGTG | |
| | LDNQKRLDSKGNI | AGTACCGCTGTTGCCACTGTCATCAA | |
| | KRCRVPLIKEAEQI | GACTAAACAAGTTGAATTCCAACTGC | |
| | AWLQRKLGNAAR | AAGTGGGCGTCCCTCTGTATTTCCGCC | |
| | VEDVHPISERPQYF | TCAGGGCCAACCCCATCAAAACCATC | |
| | SGEGKNGKIQTVCF | CTGGACAACCAGAAGCGGCTGGATAG | |
| | EGVLTINDAPALID | CAAAGGTAATATCAAGAGATGCCGCG | |
| | LLQQGIGPAKSMG | TGCCTCTGATCAAGGAGGCCGAGCAG | |
| | CGLLSLAPL(SEQ | ATCGCTTGGCTGCAACGCAAGCTGGG | |
| | ID NO: 16) | TAACGCCGCGAGAGTGGAAGATGTGC | |
| | | ACCCAATCTCCGAGCGCCCGCAGTAT | |
| | | TTCTCCGGGGAGGGGAAGAACGGCAA | |
| | | AATTCAGACTGTCTGCTTCGAGGGGG | |
| | | TGCTCACTATTAACGACGCCCCTGCTC | |
| | | TGATCGACCTCCTGCAGCAGGGCATT | |
| | | GGGCCCGCGAAGAGCATGGGATGCGG | |
| | | ATTGTTGAGCCTGGCACCCCTG (SEQ | |
| | | ID NO: 20) | |
| Cse3/Thermus | MWLTKLVLNPASR | ATGTGGTTGACCAAATTGGTTCTGAAT | GTAGTCCC |
| thermophilus | AARRDLANPYEMH | CCTGCGAGCCGCGCAGCACGGCGCGA | CACGCGTG |
| | RTLSKAVSRALEEG | TTTGGCTAACCCTTACGAGATGCATCG | TGGGGATG |
| | RERLLWRLEPARG | GACTCTTTCAAAAGCGGTTAGCAGGG | GACCG |
| | LEPPVVLVQTLTEP | CTTTGGAAGAAGGGCGCGAGCGCCTT | (SEQ ID NO: |
| | DWSVLDEGYAQVF | TTGTGGAGGCTGGAGCCAGCTCGGGG | 25) |
| | PPKPFHPALKPGQR | ACTGGAGCCCCCTGTCGTCCTGGTGC | |
| | LRFRLRANPAKRLA | AGACCCTCACTGAGCCTGATTGGTCC | |
| | ATGKRVALKTPAE | GTACTTGATGAAGGTTACGCACAGGT | |
| | KVAWLERRLEEGG | CTTTCCTCCTAAGCCTTTCCACCCAGC | |
| | FRLLEGERGPWVQI | ATTGAAGCCGGGCCAGCGGCTCCGCT | |
| | LQDTFLEVRRKKD | TTAGGCTCCGGGCGAATCCCGCCAAA | |
| | GEEAGKLLQVQAV | CGGTTGGCTGCCACCGGAAAGCGAGT | |
| | LFEGRLEVVDPERA | TGCGTTGAAAACGCCCGCCGAAAAAG | |
| | LATLRRGVGPGKA | TGGCGTGGCTTGAGAGGCGGCTGGAG | |
| | LGLGLLSVAP (SEQ | GAGGGTGGTTTTCGACTCCTTGAAGG | |
| | ID NO: 17) | GGAAAGGGGACCATGGGTACAGATAC | |
| | | TTCAAGATACGTTCCTGGAGGTGCGG | |
| | | AGAAAAAAAGACGGAGAAGAGGCAG | |
| | | GCAAGCTGCTTCAAGTCCAAGCCGTC | |
| | | TTGTTCGAGGGGAGACTCGAAGTTGT | |
| | | TGATCCTGAGAGAGCACTTGCGACAC | |
| | | TGAGACGAGGGGTGGGACCTGGTAAA | |
| | | GCGCTGGGTCTTGGACTTCTTAGTGTT | |
| | | GCACCATGA (SEQ ID NO: 21) | |

In some embodiments, the nucleotide sequence encoding the endoribonuclease is operably linked to a nucleotide sequence encoding a degradation signal. A “degradation signal” refers to a peptide sequence that mediates the degradation of the protein it is fused to. Being “operably linked,” in this contexts, means the two coding sequences are linked in frame such that when they are translated, a fusion protein comprising the endoribonuclease fused to the degradation signal is produced. The endoribonuclease, when translated, is fused to a degradation signal and is targeted for degradation (e.g., by the proteasome), thus allowing additional tuning of the antibody expression system. In some embodiments, the degradation signal is selected from: PEST, a destabilization domain from E. coli dihydrofolate reductase (ecDHFR), or a destabilization domain derived from human FKBP protein. In some embodiments, degradation of Csy4 mediated by the degradation signal is inhibited in the presence of TMP or 4-OHT.

The “PEST” sequence is a peptide sequence that is rich in proline (P), glutamic acid (E), serine (S), and threonine (T). Proteins fused to the PEST sequence is targeted for degradation by the proteasome or calpain. The PEST sequence has been described in the art, e.g., in Rogers et al., Science. 234 (4774): 364-8, 1986; Reverte et al., Dev. Biol. 231 (2): 447-58, 2001; and Spencer et al., J. Biol. Chem. 279 (35): 37069-78, 2004, incorporated herein by reference. The DHFR degradation signal and FKBP degradation signal have also been described in the art, e.g., in Rakhit et al., Bioorg Med Chem Lett. 2011 Sep. 1; 21(17): 4965-4968; and Crabb et al., PLoS ONE 7(7): e40981, 2012, incorporated herein by reference.

In some embodiments, the antibody expression system is one or more engineered viral genomes. For example, when the heavy chain and light chain are encoded on one nucleic acid, the antibody expression system contains one engineered viral genome. When the heavy chain and light chain are encoded on two nucleic acids, the antibody expression system contains two engineered viral genomes. An “engineered viral genome” refers to a viral genome engineered to incorporate the nucleic acids of the antibody expression system described herein.

In some embodiments, the viral genome is the genome of an oncolytic virus. An oncolytic virus is a virus that preferentially infects and kills cancer cells. As the infected cancer cells are destroyed by oncolysis, they release new infectious virus particles or virions to help destroy the remaining tumor. A number of viruses including adenovirus, reovirus, measles, herpes simplex, Newcastle disease virus and vaccinia have now been clinically tested as oncolytic agents (e.g., as described in Donnelly et al., Current Pharmaceutical Biotechnology. 13 (9): 1834-41, 2012, incorporated herein by reference). Most current oncolytic viruses are engineered for tumor selectivity, although there are naturally occurring examples such as reovirus and the senecavirus, resulting in clinical trials (e.g., as described in Roberts et al., Current Opinion in Molecular Therapeutics. 8 (4): 314-21, 2006; and Rudin et al., Clinical Cancer Research. 17 (4): 888-95, 2011, incorporated herein by reference). Non-limiting, exemplary oncolytic viruses include: alphaviruses, adenoviruses, reoviruses, measles virus, herpes simplex virus, Newcastle disease virus and vaccinia virus.

In some embodiments, the oncolytic virus is herpes simplex virus 1 (HSV-1). In some embodiments, the oncolytic virus is a non-replicating virus such as attenuated HSV-1. Oncolytic HSV-1 and its uses have been described in the art, e.g., in U.S. Pat. No. 9,623,059, incorporated herein by reference. HSV-1 is a double-stranded linear DNA virus in the Herpesviridae family. The structure of HSV-1 consists of a relatively large, double-stranded, linear DNA genome encased within an icosahedral protein cage called the capsid, which is wrapped in a lipid bilayer called the envelope. The envelope is joined to the capsid by means of a tegument. The complete viral particle is known as the virion (the terms “viral particle” and “virion” are used interchangeably herein).

The present disclosure also provides viral particles comprising the antibody expression system described herein. The viral particles may be produced by packaging cells. For viral particle packaging, the nucleic acids of the antibody expression system described herein are delivered to a packaging cell, e.g., via any methods known to those skilled in the art, such as transfection or electroporation.

Any cells suitable for viral particle packaging may be used as the packaging cell of the present disclosure. In some embodiments, the packaging cell is an eukaryotic cell. Examples of eukaryotic cells for use in accordance with the invention include, without limitation, mammalian cells, insect cells, yeast cells (e.g., Saccharomyces cerevisiae) and plant cells. In some embodiments, the eukaryotic cells are from a vertebrate animal. In some embodiments, the cell is a mammalian cell. In some embodiments, the cell is a human cell. In some embodiments, the cell is from a rodent, such as a mouse or a rat. Examples of vertebrate cells for use in accordance with the present disclosure include, without limitation, reproductive cells including sperm, ova and embryonic cells, and non-reproductive cells, including kidney, lung, spleen, lymphoid, cardiac, gastric, intestinal, pancreatic, muscle, bone, neural, brain and epithelial cells. Stem cells, including embryonic stem cells, can also be used. Typically, it is preferably to use cell lines that have high transfection efficiency and low innate immune response for high viral titer production. In some embodiments, the packaging cell is a U2OS cell (ATCC® HTB-96™).

In some embodiments, the antibody expression system is one or more Minicircle DNA molecules. A “minicircle DNA” is a small (˜4 kb) circular plasmid derivatives that have been freed from all prokaryotic vector parts. Minicircle DNAs have been applied as transgene carriers for the genetic modification of mammalian cells, with the advantage that, since they contain no bacterial DNA sequences, they are less likely to be perceived as foreign and destroyed. The smaller size of minicircles also extends their cloning capacity and facilitates their delivery into cells.

The preparation of minicircle DNAs have been described in the art (e.g., in Nehlsen et al., Gene Ther. Mol. Biol. 10: 233-244, 2006; and Kay et al., Nature Biotechnology. 28: 1287-1289, 2010, incorporated herein by reference. The preparation generally usually follows a two-step procedure: (i) production of a ‘parental plasmid’ (bacterial plasmid with eukaryotic inserts) in E. coli; and (ii) induction of a site-specific recombinase at the end of this process but still in bacteria. These steps are followed by the excision of prokaryotic vector parts via two recombinase-target sequences at both ends of the insert to recover of the resulting minicircle by capillary gel electrophoresis.

The antibody expression system of the present disclosure (e.g., either in the form of viral genomes or minicircle DNAs) may be delivered to a cell by any methods familiar to those skilled in the art (e.g., without limitation, transformation, transfection, and electroporation).

Cells containing the antibody expression system are also provided herein. A “cell” is the basic structural and functional unit of all known independently living organisms. It is the smallest unit of life that is classified as a living thing. Some organisms, such as most bacteria, are unicellular (consist of a single cell). Other organisms, such as humans, are multicellular.

In some embodiments, a cell for use in accordance with the present disclosure is a prokaryotic cell, which may comprise a cell envelope and a cytoplasmic region that contains the cell genome (DNA) and ribosomes and various sorts of inclusions. In some embodiments, the cell is a bacterial cell. As used herein, the term “bacteria” encompasses all variants of bacteria, for example, prokaryotic organisms and cyanobacteria. Bacteria are small (typical linear dimensions of around 1 micron), non-compartmentalized, with circular DNA and ribosomes of 70S. The term bacteria also includes bacterial subdivisions of Eubacteria and Archaebacteria. Eubacteria can be further subdivided into gram-positive and gram-negative Eubacteria, which depend upon a difference in cell wall structure. Also included herein are those classified based on gross morphology alone (e.g., cocci, bacilli). In some embodiments, the bacterial cells are gram-negative cells, and in some embodiments, the bacterial cells are gram-positive cells. Examples of bacterial cells that may be used in accordance with the invention include, without limitation, cells from Yersinia spp., Escherichia spp., Klebsiella spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Salmonella spp., Vibrio spp., Bacillus spp., Erysipelothrix spp., Salmonella spp., Stremtomyces spp. In some embodiments, the bacterial cells are from Staphylococcus aureus, Bacillus subtilis, Clostridium butyricum, Brevibacterium lactofermentum, Streptococcus agalactiae, Lactococcus lactis, Leuconostoc lactis, Streptomyces, Actinobacillus actinobycetemcomitans, Bacteroides, cyanobacteria, Escherichia coli, Helobacter pylori, Selnomonas ruminatium, Shigella sonnei, Zymomonas mobilis, Mycoplasma mycoides, Treponema denticola, Bacillus thuringiensis, Staphylococcus lugdunensis, Leuconostoc oenos, Corynebacterium xerosis, Lactobacillus planta rum, Streptococcus faecalis, Bacillus coagulans, Bacillus ceretus, Bacillus popillae, Synechocystis strain PCC6803, Bacillus liquefaciens, Pyrococcus abyssi, Selenomonas nominantium, Lactobacillus hilgardii, Streptococcus ferus, Lactobacillus pentosus, Bacteroides fragilis, Staphylococcus epidermidis, Zymomonas mobilis, Streptomyces phaechromogenes, Streptomyces ghanaenis, Halobacterium strain GRB, or Halobaferax sp. strain Aa2.2.

In some embodiments, a cell for use in accordance with the present disclosure is a eukaryotic cell, which comprises membrane-bound compartments in which specific metabolic activities take place, such as a nucleus. Examples of eukaryotic cells for use in accordance with the invention include, without limitation, mammalian cells, insect cells, yeast cells (e.g., Saccharomyces cerevisiae) and plant cells. In some embodiments, the eukaryotic cells are from a vertebrate animal. In some embodiments, the cell is a mammalian cell. In some embodiments, the cell is a human cell. In some embodiments, the cell is from a rodent, such as a mouse or a rat. Examples of vertebrate cells for use in accordance with the present disclosure include, without limitation, reproductive cells including stem cells, sperm, ova and embryonic cells, and non-reproductive cells, including kidney, lung, spleen, lymphoid, cardiac, gastric, intestinal, pancreatic, muscle, bone, neural, brain and epithelial cells. In some embodiments, the stem cells are induced pluripotent stem cells (iPSC).

In some embodiments, the cell is a diseased cell. A “diseased cell,” as used herein, refers to a cell whose biological functionality is abnormal, compared to a non-diseased (normal) cell. In some embodiments, the diseased cell is a cancer cell.

In some embodiments, the cell is an immune cell. Non-limiting examples of immune cells include: antigen-presenting cell, dendritic cell, monocyte, macrophage, NKT cell, NK cell, basophil, eosinophil, or neutrophil, B cell, T cell (CD4 or CD8), regulatory T cell, antigen-presenting cell, dendritic cell, monocyte, macrophage, NKT cell, NK cell, basophil, eosinophil, and neutrophil. In some embodiments, the T cells are naïve or activated T cells.

Other aspects of the present disclosure provide methods of producing antibodies using the antibody expression system described herein. In some embodiments, the antibodies are produced in prokaryotic cells (e.g., bacterial cells). In some embodiments, the antibodies are produced in eukaryotic cells (e.g., yeast cells, insect cells, or mammalian cells). Mammalian host cells for expressing the antibodies or antigen-binding fragments thereof include Chinese Hamster Ovary (CHO cells) (including dhfr− CHO cells, described in Urlaub and Chasin, 1980, Proc. Natl. Acad. Sci. USA 77:4216-4220, used with a DHFR selectable marker, e.g., as described in Kaufman and Sharp, 1982, Mol. Biol. 159:601 621), lymphocytic cell lines, e.g., NSO myeloma cells and SP2 cells, COS cells, and a cell from a transgenic animal, e.g., a transgenic mammal.

In addition to the nucleic acids in the antibody-expression system, other nucleic acids may be introduced to the host cell, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr− host cells with methotrexate selection/amplification) and the neo gene (for G418 selection).

In an exemplary system for recombinant expression of an antibody, or antigen-binding portion thereof, a recombinant expression vector encoding both the antibody heavy chain and the antibody light chain is introduced into dhfr− CHO cells by calcium phosphate-mediated transfection.

In some embodiments, if the RNA replicon is under the control of an inducible promoter, the method of producing antibodies further comprises providing the cells with an inducer that activates the inducible promoter. The host cells for expressing the antibody may be in vitro, in vivo, or ex vivo.

In some embodiments, the host cell is in vivo, e.g., in a subject such as a human subject. The present disclosure thus contemplates methods of expressing therapeutic antibodies in a subject (e.g., a human subject) for treating a disease, the method comprising administering to a subject in need thereof an effective amount of the antibody expression system or the viral particle comprising the antibody expression system described herein.

For administration to a subject, the antibody expression system or the viral particle comprising the antibody expression system may be formulated in a composition. In some embodiments, the composition is a pharmaceutical composition. In some embodiments, the composition further comprises additional agents (e.g. for specific delivery, increasing half-life, or other therapeutic agents). In some embodiments, the composition further comprises a pharmaceutically acceptable carrier. The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. A “pharmaceutically acceptable carrier” is a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

Some examples of materials which can serve as pharmaceutically-acceptable carriers include, without limitation: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as peptides and amino acids (23) serum component, such as serum albumin, HDL and LDL; (24) C2-C12 alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient,” “carrier,” “pharmaceutically acceptable carrier” or the like are used interchangeably herein.

The pharmaceutical compositions described herein to be used in the present methods can comprise pharmaceutically acceptable carriers, buffer agents, excipients, salts, or stabilizers in the form of lyophilized formulations or aqueous solutions. See, e.g., Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some embodiments, the pharmaceutical composition described herein comprises lipid nanoparticles which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

An “effective amount” refers to the amount of the engineered the antibody expression system or the viral particle comprising the antibody expression system required to confer therapeutic effect on the subject, either alone or in combination with one or more other therapeutic agents. Effective amounts vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons or for virtually any other reasons.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of a disorder. Alternatively, sustained continuous release formulations of agent may be appropriate. Various formulations and devices for achieving sustained release are known in the art.

An effective amount of the antibody expression system or the viral particle comprising the antibody expression system may be administered repeatedly to a subject (e.g., 2, 3, 4, 5, 6, 7, 8, 9, 10 times or more). In some embodiments, dosage is daily, every other day, every three days, every four days, every five days, or every six days. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the agents used) can vary over time.

In some embodiments, for an adult subject of normal weight, doses ranging from about 0.01 to 1000 mg/kg may be administered. In some embodiments, the dose is between 1 to 200 mg. The particular dosage regimen, i.e., dose, timing and repetition, will depend on the particular subject and that subject's medical history, as well as the properties of the agent (such as the half-life of the agent, and other considerations well known in the art).

For the purpose of the present disclosure, the appropriate dosage of the antibody expression system or the viral particle comprising the antibody expression system as described herein will depend on the specific agent (or compositions thereof) employed, the formulation and route of administration, the type and severity of the disorder, previous therapy, the subject's clinical history and response to the agents, and the discretion of the attending physician. Typically the clinician will administer an agent until a dosage is reached that achieves the desired result. Administration can be continuous or intermittent, depending, for example, upon the recipient's physiological condition, and other factors known to skilled practitioners. The administration of an agent may be essentially continuous over a preselected period of time or may be in a series of spaced dose, e.g., either before, during, or after developing a disorder.

A “subject” refers to human and non-human animals, such as apes, monkeys, horses, cattle, sheep, goats, dogs, cats, rabbits, guinea pigs, rodents (e.g., rats, and mice). In one embodiment, the subject is human. In some embodiments, the subject is an experimental animal or animal substitute as a disease model. A “subject in need thereof” refers to a subject who has or is at risk of a disease or disorder (e.g., cancer).

The antibody expression system or the viral particle comprising the antibody expression system may be delivered to a subject (e.g., a mammalian subject, such as a human subject) by any in vivo delivery method known in the art. In some embodiments, the antibody expression system, the expression system, or the viral particle can be delivered to a subject by electroporation. In some embodiments, the antibody expression system or the viral particle comprising the antibody expression system may be delivered intravenously. In some embodiments, engineered nucleic acids are delivered in a delivery vehicle (e.g., non-liposomal nanoparticle or liposome). In some embodiments, the antibody expression system or the viral particle comprising the antibody expression system is delivered systemically to a subject having a cancer or other disease and produces a therapeutic molecule specifically in cancer cells or diseased cells of the subject. In some embodiments, the antibody expression system or the viral particle comprising the antibody expression system is delivered locally to a site of the disease or disorder (e.g., site of cancer).

Various diseases may be treated using the compositions and methods described herein. In some embodiments, the disease is a disease that can be treated by gene therapy. One skilled in the art is familiar with such diseases. In some embodiments, the disease is cancer.

Non-limiting examples of cancers that may be treated using the compositions and methods described herein include: premalignant neoplasms, malignant tumors, metastases, or any disease or disorder characterized by uncontrolled cell growth such that it would be considered cancerous or precancerous. The cancer may be a primary or metastatic cancer. Cancers include, but are not limited to, ocular cancer, biliary tract cancer, bladder cancer, pleura cancer, stomach cancer, ovary cancer, meninges cancer, kidney cancer, brain cancer including glioblastomas and medulloblastomas, breast cancer, cervical cancer, choriocarcinoma, colon cancer, endometrial cancer, esophageal cancer, gastric cancer, hematological neoplasms including acute lymphocytic and myelogenous leukemia, multiple myeloma, AIDS-associated leukemias and adult T-cell leukemia lymphoma, intraepithelial neoplasms including Bowen's disease and Paget's disease, liver cancer, lung cancer, lymphomas including Hodgkin's disease and lymphocytic lymphomas, neuroblastomas, oral cancer including squamous cell carcinoma, ovarian cancer including those arising from epithelial cells, stromal cells, germ cells and mesenchymal cells, pancreatic cancer, prostate cancer, rectal cancer, sarcomas including leiomyosarcoma, rhabdomyosarcoma, liposarcoma, fibrosarcoma, and osteosarcoma, skin cancer including melanoma, Kaposi's sarcoma, basocellular cancer, and squamous cell cancer, testicular cancer including germinal tumors such as seminoma, non-seminoma, teratomas, choriocarcinomas, stromal tumors and germ cell tumors, thyroid cancer including thyroid adenocarcinoma and medullar carcinoma, and renal cancer including adenocarcinoma and Wilms' tumor. Commonly encountered cancers include breast, prostate, lung, ovarian, colorectal, and brain cancer. In some embodiments, the tumor is a melanoma, carcinoma, sarcoma, or lymphoma. In some embodiments, the cancer is breast cancer, glioblastoma, pancreatic cancer, prostate cancer, or lung cancer.

For treating cancer, the antibody expression system, when delivered to a subject in need thereof, expresses antibodies that have anti-cancer therapeutic effects. In some embodiments, the anti-cancer antibody is an immune checkpoint inhibitor (e.g., any of the immune checkpoint inhibitors described herein and known in the art).

Other aspects of the present disclosure provide methods of producing the antibody expression system described herein. In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies.

Further, as illustrated in Example 3, the antibody expression system is constructed using a strategy similar to the MoClo system (e.g., as described in Weber et al., PLoS ONE 6(2): e16765, 2011, incorporated herein by reference). In some embodiments, the method of producing the antibody expression system comprises:

(i) providing a plurality of genetic elements comprising a plurality of viral subgenomic viral promoters, a nucleotide sequence encoding an immunoglobulin heavy chain, a nucleotide sequence encoding an immunoglobulin light chain, and optionally a nucleotide sequence encoding a 3′ untranslated region (3′UTR), wherein each genetic element is flanked at the 3′ end and the 5′ end by a recognition and cleavage site for a first type IIS restriction endonuclease, and wherein the recognition and cleavage site is engineered to allow directional assembly of the plurality genetic elements;

(ii) assembling a first transcriptional unit comprising, in order from 5′ to 3′, a first subgenomic viral promoter, the nucleotide sequence encoding the immunoglobulin heavy chain, and optionally the nucleotide sequence encoding the 3′UTR, by combining the genetic elements with:
(a) the first type IIS restriction endonuclease;
(b) a ligase; and
(c) a first destination vector comprising a pair of the recognition and cleavage sites for the first type IIS restriction endonuclease and a pair of the recognition and cleavage sites for a second type IIS restriction endonuclease, wherein the pair of recognition and cleavage sites for the second type IIS restriction endonuclease enclose the pair of recognition and cleavage sites for the first type IIS restriction endonuclease, and wherein the two pairs of recognition and cleavage sites are positioned in inverse orientation relative to each other;
wherein the contacting is carried out under conditions that allow the cleavage at the recognition and cleavage sites for the first type IIS restriction endonuclease and the ligation of resulting fragments in a directional manner;

(iii) assembling a second transcriptional unit comprising, in order from 5′ to 3′, a second subgenomic viral promoter, the nucleotide sequence encoding the immunoglobulin light chain, and optionally the nucleotide sequence encoding the 3′UTR, by contacting the genetic elements with:
(a) the first type IIS restriction endonuclease;
(b) a ligase; and
(c) a first destination vector comprising a pair of the recognition and cleavage sites for the first type IIS restriction endonuclease and a pair of the recognition and cleavage sites for a second type IIS restriction endonuclease, wherein the pair of recognition and cleavage sites for the second type IIS restriction endonuclease enclose the pair of recognition and cleavage sites for the first type IIS restriction endonuclease, and wherein the two pairs of recognition and cleavage sites are positioned in inverse orientation relative to each other;
wherein the contacting is carried out under conditions that allow the cleavage at the recognition and cleavage sites for the first type IIS restriction endonuclease and the ligation of resulting fragments in a directional matter;

(iv) assembling the antibody expression system by combining the first transcriptional unit obtained in (ii) and the second transcriptional unit obtained in (iii) with:
(a) the second type IIS restriction endonuclease;
(b) a ligase; and
(c) a second destination vector comprising a promoter operably linked to a nucleotide sequence encoding one or more viral non-structural proteins, and a pair of the recognition and cleavage sites for the second type IIS restriction endonuclease,
wherein the contacting is carried out under conditions that allow the cleavage at the recognition and cleavage sites for the second type IIS restriction endonuclease and the ligation of resulting fragments in a directional manner.

In some embodiments, the first type IIS restriction endonuclease is BsaI. In some embodiments, the second type IIS restriction endonuclease is SapI. One skilled in the art is able to choose appropriate restriction endonucleases for constructing the antibody expression system described herein. In some embodiments, the immunoglobulin is an immune checkpoint inhibitor, e.g., any of the immune checkpoint inhibitors described herein or known in the art.

### EXAMPLES

Transgene expression directly influences efficacy of therapy. Prolonged, high level expression of transgenes is desired in therapeutic applications such as vaccination and cancer immunotherapy. However, it has been difficult to achieve such expression profile using traditional expression cassettes driven by pol-II promoters. Recently there has been an effort to encode transgenes on RNA replicon, which can amplify signals. RNA replicon is a modified alphavirus that harness amplification of RNA viral genome to overexpress its transgene payload. Here, the power of RNA replicon by encoding on DNA is further expanded. It allows for fine-control timing of the launch of RNA replicon using transcription and translational control. Using the previously filed sub-genomic promoter library, the expression of antibody was optimized by controlling the ratio between heavy and light chains expression.

### Example 1. DNA Virus Launched RNA Replicon (VREP)

Production and delivery of RNA replicons can be a challenge in therapeutic applications. Most common way to produce RNA replicons is by in vitro transcription in a test tube. Due to lack of proof reading mechanism in RNA polymerases, unwanted mutations can be accumulated during RNA amplification. Additionally, RNA stability in vivo required packaging into polymer or nanoparticle carriers. However, this process is difficult to scale up and delivery efficiency in vivo still needs substantial improvements.

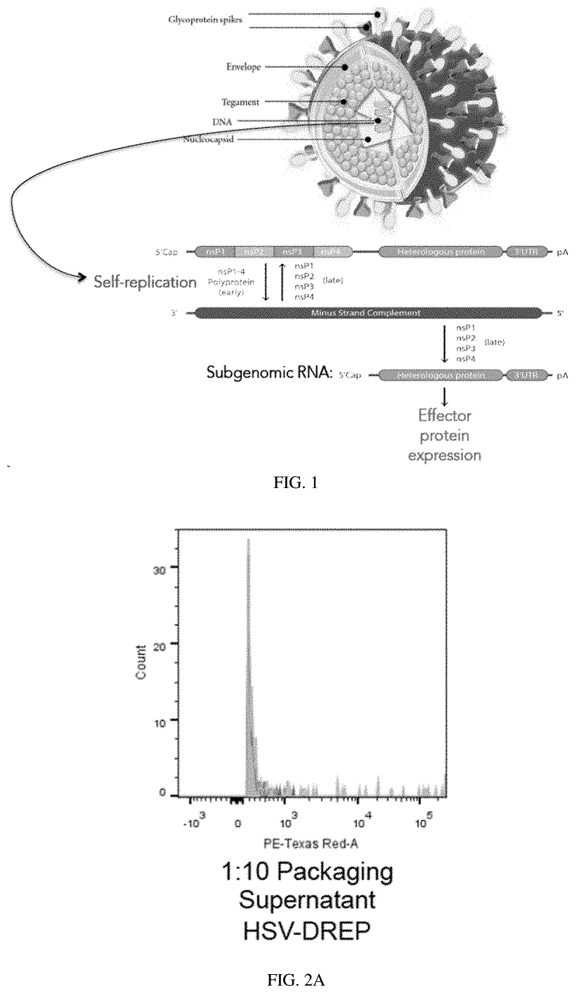

Provided herein are DNA virus launched RNA replicons (VREPs) in which DNA encoding RNA replicon (DREP) is packaged into DNA viral vectors (FIG. 1). DREP is encoded in HSV-1 genome and packaged into virions. Upon transcription of DREP, RNA replicon consisted of 5′ Cap, nsP1-4, heterologous protein, 3′ UTR, and polyA is generated and self-replicated. Subgenomic RNA is then produced from subgenomic promoter and effector protein is translated. DREP can be scaled up in a plasmid form and has lower rate of mutation by bypassing the need of in vitro transcription. Viruses have evolved to efficiently enter the target cells and deliver their genetic payload to the nucleus. By packaging DREP into DNA virus, DREP can be efficiently delivered to the target cells and express RNA replicon. Utilization of DNA virus is more advantageous than RNA virus such as alphavirus which RNA replicon is derived from, due to lower rate of accumulation of mutation in DNA replication.

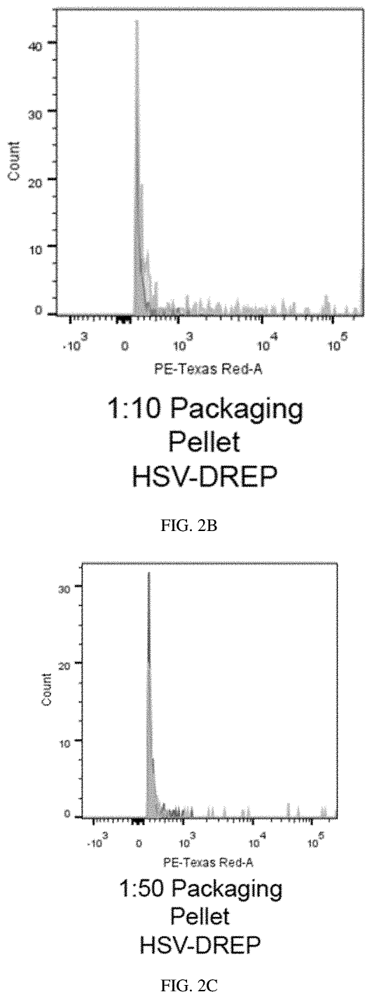

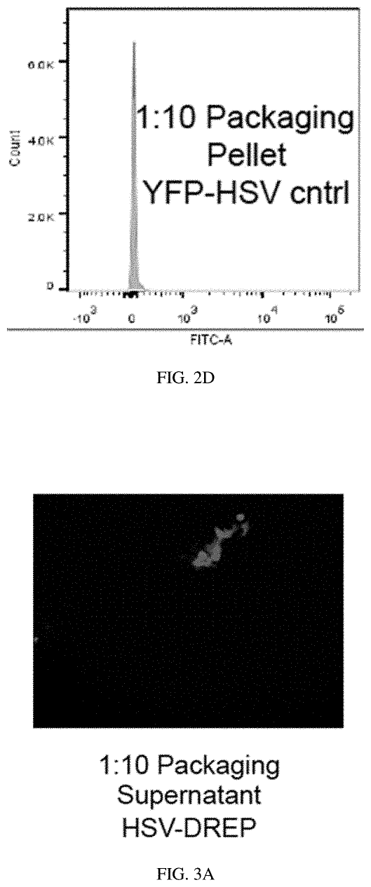

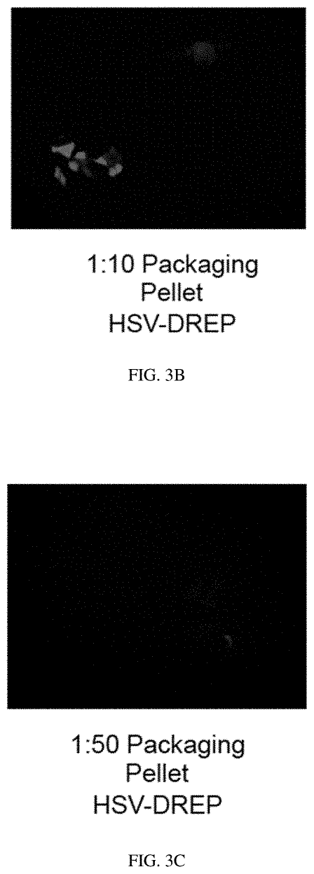

To demonstrate mKate expressing, DREP were packaged into HSV-1 genome and delivered to target cells, nsP1234-mKate-3′UTR-polyA sequence was integrated in HSV-1 genome. As a control, hEF1a driving EYFP was integrated in HSV-1 genome. HSV-1 genomes were purified from E. coli and transfected into the packaging cell line in 6-well plate to produce infectious virions. When packaging cells showed over 50% cytopathic effect, cell pellets and supernatant were collected separately, and cell pellets were further treated by three cycles of freeze/thaw to harvest virions. 1:10 and 1:50 of harvested virions either from cell pellet or supernatant were used to infect U2OS cells. With VREP infected cells, more cell counts showed higher expression of mKate (FIGS. 2A-2C), while non-infected cells were shown in red. hEF1a-EYFP carrying HSV-1 infected cells did not express any significant level of EYFP above background (FIG. 2D). Corresponding images were taken by EVOS microscope (FIGS. 3A-3C).

To validate whether DREP can be launched by a cell classifier, a full classifier driving expression of RNA replicon as an output was encoded in plasmid DNA (FIG. 4A). Plasmid DNA was transfected into HEK and Vero cells in which their miRNA profile resembles Off and On-state, respectively. The performance of the DREP cell classifier was measured by flow cytometry. DREP classifier circuits were further optimized and then integrated into HSV-1 genome to test whether functional DREP classifier circuit can be delivered by HSV-1 and express the output in high level only if the miRNA profile of the target cell line is matching.

### Example 2. Optimization of DNA Launched Replicons (DREP)

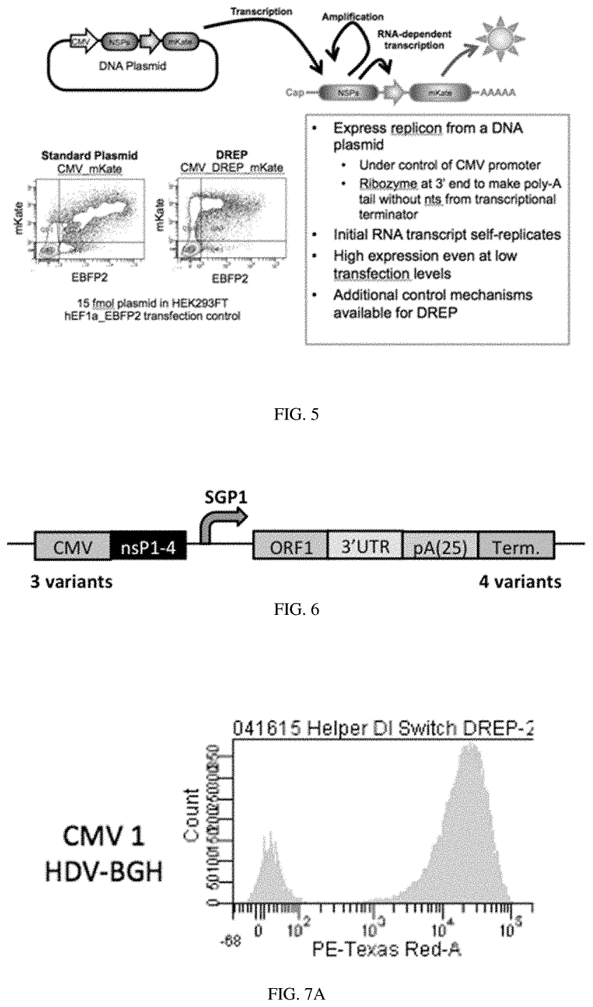

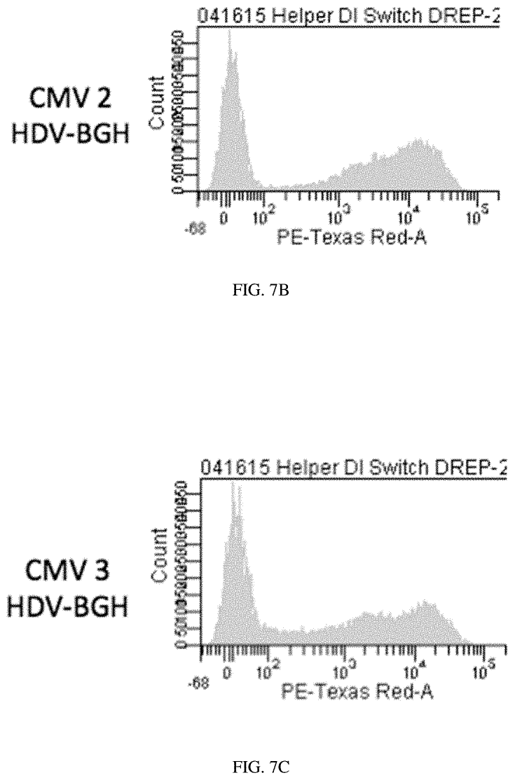

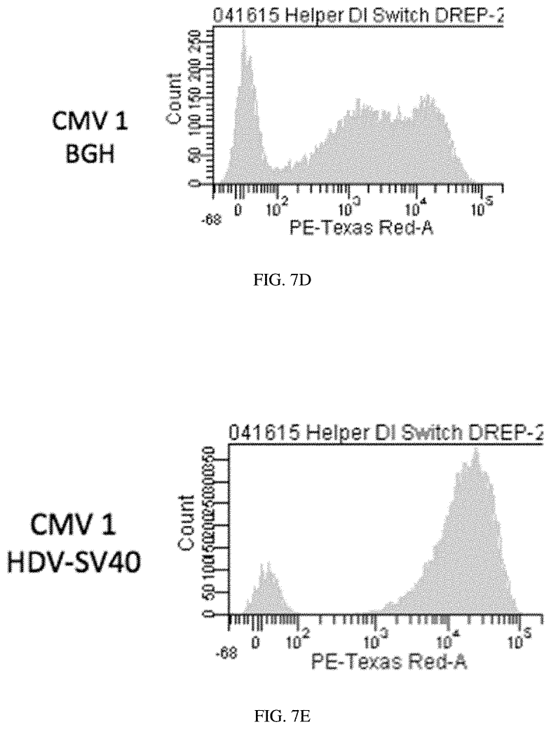

After RNA replicon is transcribed from DNA template, capped RNA strands go through series of amplification and transcription (FIG. 5). Upon translation, P1234 is rapidly cleaved into P123 and nsP4 by autoproteolytic activity originating from the nsP2 (proteinase) portion of the polyprotein. Alphaviral RNA synthesis occurs at the plasma membrane of a cell, where the nsPs, together with alphaviral RNA, form membrane invaginations (or “spherules”). These spherules contain dsRNA created by replication of “+” strand viral genomic RNA into “−” strand anti-genomic RNA. The “−” strand serves as a template from which additional “+” strand genomic RNA (synthesized from the 5′UTR) or a shorter subsequence of the genomic RNA (termed subgenomic RNA) is synthesized from the subgenomic viral promoter region located near the end of the nonstructural protein ORF. The “+” strand genomic RNA and the subgenomic RNA are exported out of the spherules into the cytoplasm where they are translated by endogenous ribosomes. The exported “+” strand genomic RNA can associate with nsPs and form additional spherules, thus resulting in exponential increase of replicon RNA. 5′ cap has turned out to be important in this process, and changes in location and sequence of the transcription start site can result in significant decrease in transgene expression from RNA replicon. In addition, 3′ end of RNA replicon can also affect transgene expression level. Therefore, three variants of CMV promoters and three variants of the terminator sequence were tested (FIG. 6). A combination of promoters, CMV 1, CMV 2, and CMV 3, and terminator sequences, HDV-BGH, BGH, and HDV-SV40, were constructed in plasmid DNA. Each of them was transfected into HEK293 cells and expression of transgene, mKate, was measured 48 hours post transfection by flow cytometry (FIG. 7). CMV 1 with HDV-BGH showed single population of cells with high mKate expression compared to CMV 2 with HDV-BGH and CMV 3 with HDV-BGH (FIGS. 7A-7C). CMV 1 with HDV-BGH and CMV-1 with HDV-SV40 showed single population of cells with high mKate expression compared to CMV 1 with BGH, confirming HDV is required for better performance (FIGS. 7A, 7D-7E).

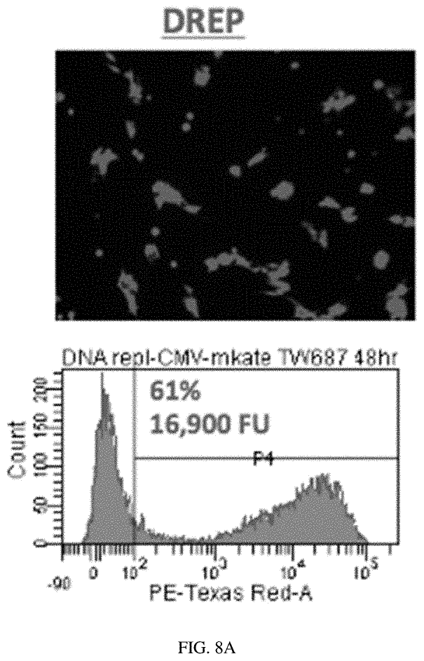

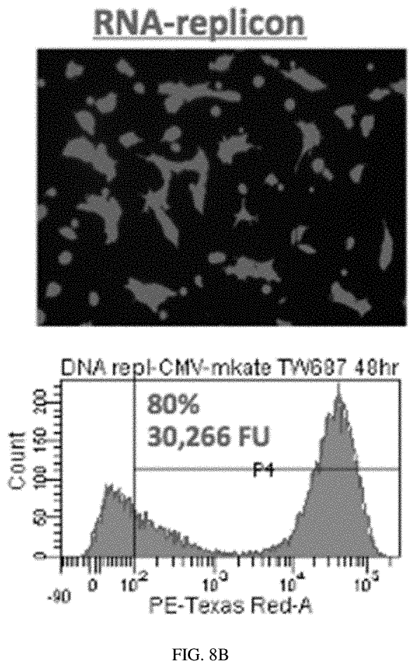

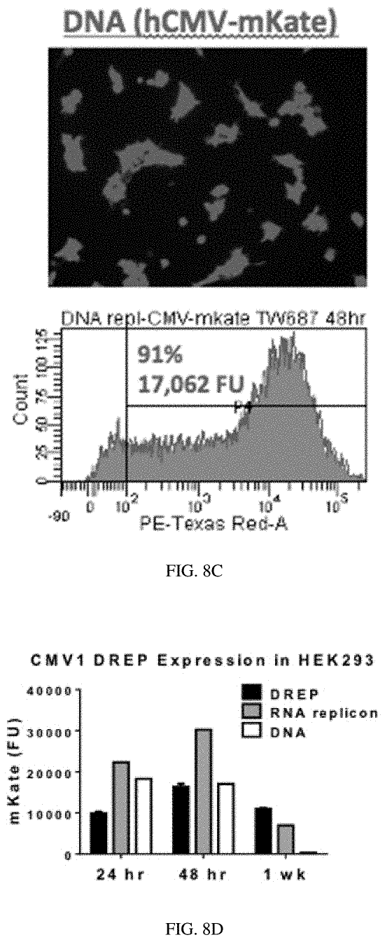

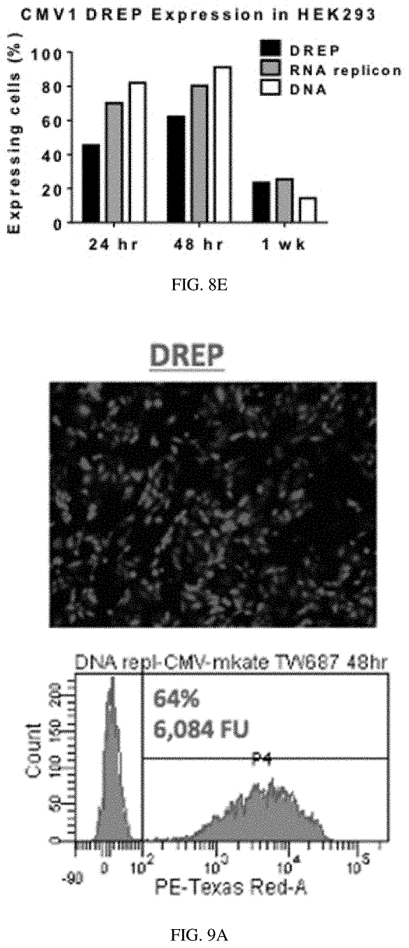

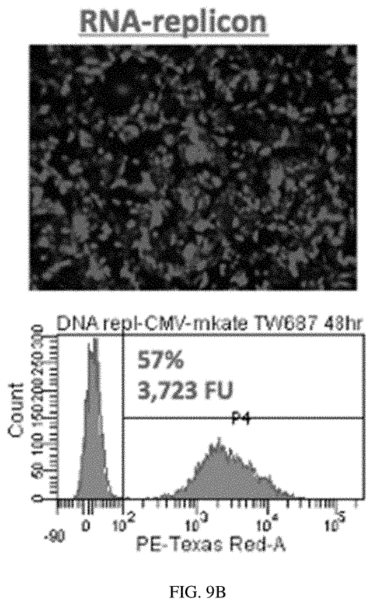

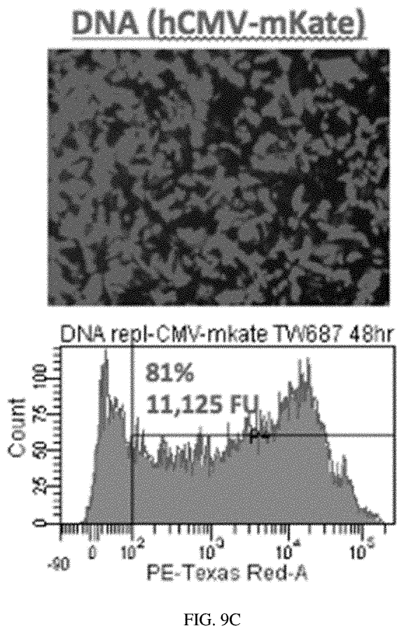

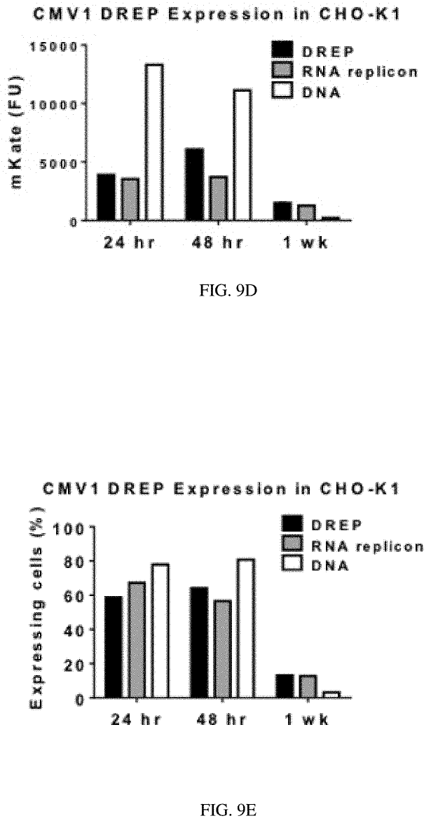

Next, to compare expression profile between DREP and RNA replicon, CMV 1 with HDV-BGH, RNA replicon produced by in vitro transcription, or plasmid DNA encoding hCMV-mKate was transfected into HEK293 cells, and mKate expression profile was measured 48 hours post transfection using flow cytometry. Although mKate expression from was slightly slower than that of RNA replicon, it was significantly higher than that of plasmid DNA (FIGS. 8A-8C). Duration of mKate expression from DREP, RNA replicon, or plasmid DNA was also measured 24 hours, 48 hours, and 1 week post transfection in HEK293. Lacking amplification of the mKate carrier, mKate expression from plasmid DNA was significantly reduced after 1 week, where mKate expression from DREP and RNA replicon remained significantly high even after 1 week (FIG. 8D). The number of mKate expressing cells was growing until 48 hours post transfection and then reduced after 1 week (FIG. 8E). To determine whether this trend is HEK293 specific, the same experimental setting was repeated in CHO-K1 cells, and the similar trend was confirmed in CHO-K1 (FIGS. 9A-9E).

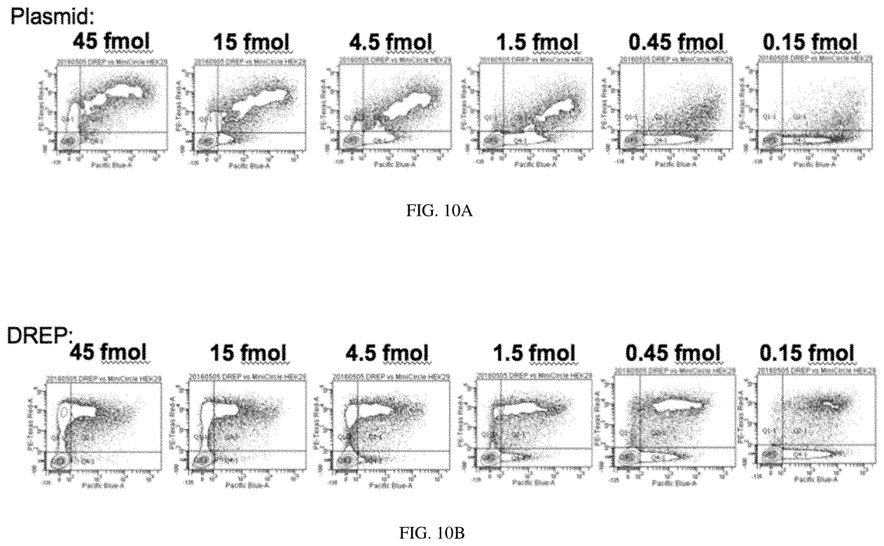

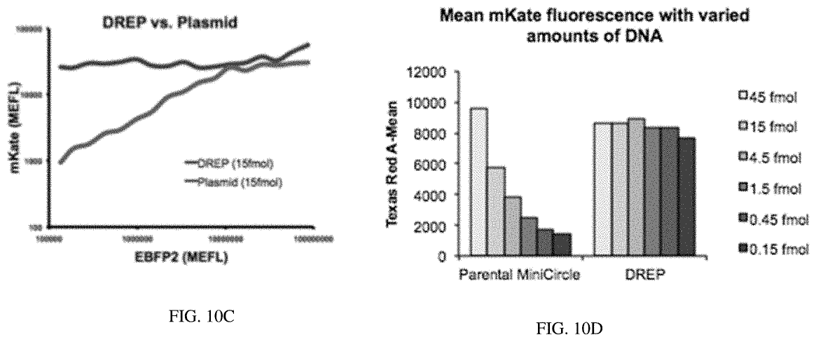

One of the advantage of DREP is that smaller number of RNA replicon launched from DREP can self-amplify and express very high amount of transgene. As a proof of concept, mKate expression from DREP was compared to that from plasmid DNA. mKate expressing DREP was transferred to minicircle DNA. Either plasmid DNA or DREP is co-transfected with a plasmid expressing EBFP2 so that DNA copy number per cell can be estimated by EBFP2 signal. As expected, DREP shows high expression profile even when plasmid copy number in a given cell is low (FIGS. 10B-10D).

### Example 3. MoClo Construction and Different Genetic Elements

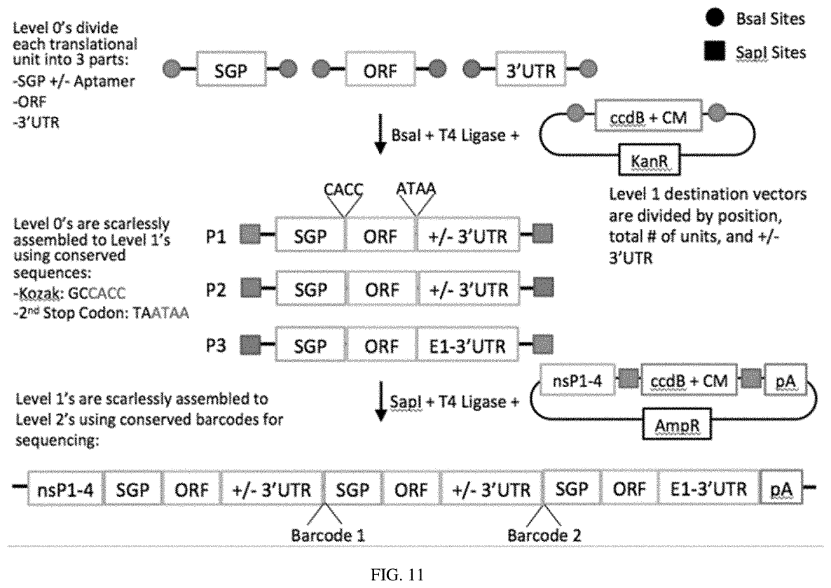

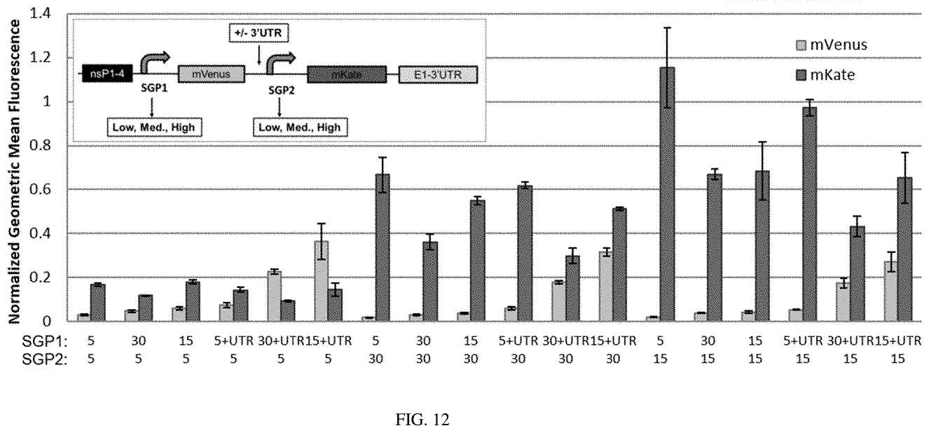

After previously determining the elements governing expression from multi-SGP systems, namely position, SGP strength, and the presence of additional 3′UTR sequences, constitutive expression from two and three SGP replicons using fluorescent reporters was characterized. It became clear that such characterization could not be completed without a high-throughput workflow, so a Modular Cloning (MoClo) assembly strategy was adapted for VEE replicons. As shown in FIG. 11, each translational unit was divided into three parts: a sub-genomic promoter (SGP), open reading frame (ORF), and 3′-untranslated region (3′UTR). Each of the parts was placed in a Level 0 vector and flanked by BsaI recognition sites. BsaI, a Type IIS restriction enzyme, recognizes a sequence and cleaves downstream of it recognition site, allowing for scarless assembly. The Level 0's were combined into a Level 1 vector to form a single translational unit, using conserved sequences in between the SGP, ORF, and 3′UTR. Finally, Level 1's were combined into the replicon backbone using a second Type IIs enzyme, SapI, to form the final Level 2 product, a functional multi-unit replicon. This assembly strategy was extremely efficient, with respect to both reaction time (˜1.25 hours for each step) and percentage of correct clones (˜75% correct by picking one colony, ˜100% correct by picking 3 colonies), and was used to generate the majority of the multi-SGP replicons discussed in this report.

Using this MoClo-based cloning strategy, all combinations of two and three SGP constructs containing low (SGPS), midrange (SGP30), and high (SGP15) subgenomic viral promoter strengths were generated, with and without additional 3′UTRs. FIG. 12 shows the results for the two SGP configuration in BHK-21 cells, with mVenus expressed under the first SGP and mKate expressed under the second SGP. If the SGPs are identical and there is not an additional 3′UTR in between the translational units, then expression from the second translational unit is between 5- and 10-fold higher than the first. As shown, this difference in expression can be mitigated by strengthening the first SGP, weakening the second SGP, and by inserting an additional 3′UTR.

These results also indicate an additional parameter with a lesser impact on expression: SGP length. The results for mVenus expression from the first SGP behaved as expected, with a systematic increase in expression from the weak SGP5 to the strong SGP15, and slightly higher expression of each after including another 3′UTR. While mKate expression showed this same general increase from SGP5 to SGP15 under the second SGP, the first SGP in front of mVenus also affected mKate expression, but not in a strength-dependent manner. Higher mVenus expression may take resources away, leading to slightly lower mKate expression. However, when holding the second SGP constant, mKate expression is inversely correlated to the length of the first SGP. Replicon position, additional 3′UTRs, and SGP choice are most important when determining expression level (in that order), but how combinations of SGPs can affect one another must also be considered.

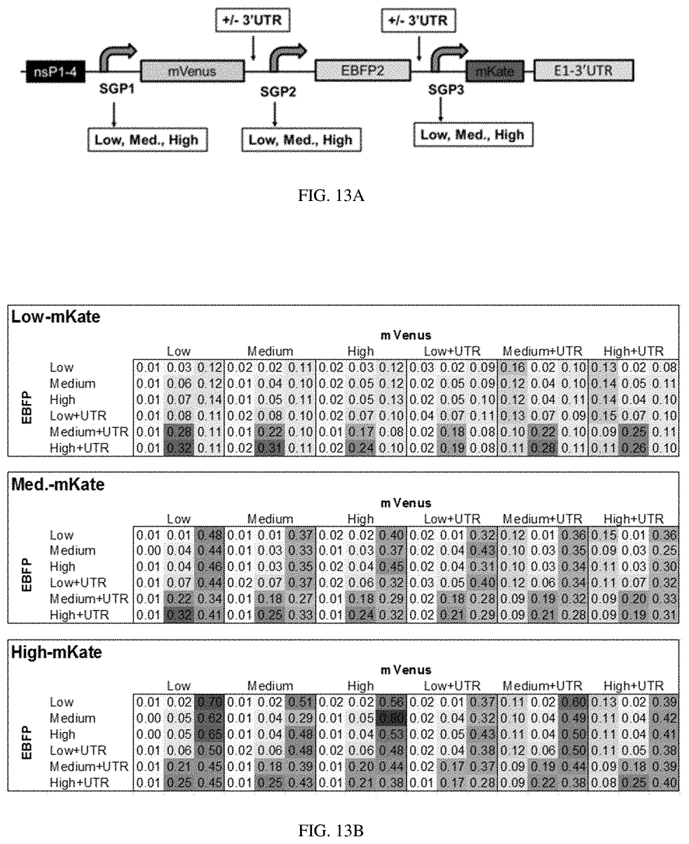

Constructs with three SGPs were created to validate the results observed with two SGPs, as shown in FIGS. 13A-13B. Fluorescence was normalized against single SGP controls expressing each fluorescent protein under the wild type subgenomic viral promoter. As expected, the third translational unit dominated expression. Modulating SGP strength and introducing additional 3′UTR sequences can be used to control expression only to a certain extent. The influence of the first SGP length on subsequent SGPs becomes inconsequential. Presumably, as more SGPs are added, expression from the 5′-most translational units will continue to decline, limiting the scalability of this approach, depending on the necessary expression levels required for circuit function. In the future, this constitutive characterization data may be combined with more comprehensive RBP characterization to create predictive expression models and direct more complex circuit design.

### Example 4. Minicircle DNAs

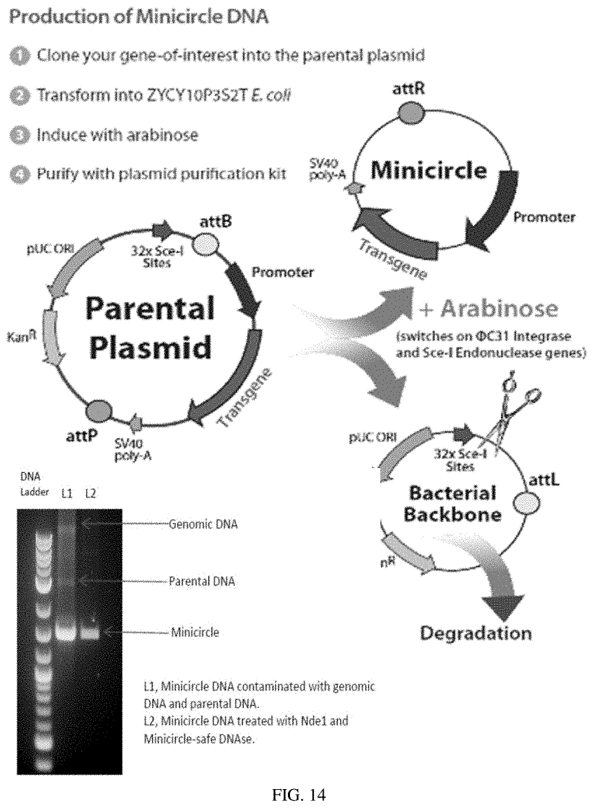

Minicircles (mc) are small DNA vectors that no longer contain antibiotic resistance markers or the bacterial origin of replication. Mc production occurs in vivo in an engineered E. coli producer strain that harbors an arabinose-inducible system to express the PhiC31 integrase and I-SceI endonuclease. Upon the induction mc-DNA vectors are generated from parental plasmids via intramolecular recombination while the residual parental vector and the excised bacterial backbone are degraded by I-SceI endonuclease (Kay M A, et al. Nat Biotechnol 2010) (FIG. 14). Minicircles can be used in vitro and in vivo and allow enhanced and prolonged transient expression compared to regular plasmids probably by eliminating heterochromatin formation induced by the plasmid backbone and methylation and transgene silencing. Due to these beneficial properties minicircle technology became the system of choice when long term transgene expression in cells and tissues is required, including antibody expression in DNA vaccination.

Self-replicating RNA is another system that offers enhanced and prolonged transgene expression without the risk of epigenetic silencing observed with DNA. Yet, efficiency of current intramuscular RNA delivery methods in vivo is significantly lower than their DNA counterparts. The DNA-launched replicon (DREP) system can successfully overcome these issues. The DREP is based on non-cytopathic VEE replicon (nsP2 Q739L; Petrakova 0 et al. J Virol. 2005) that has significantly reduced cellular toxicity and is considered safer to use than previous cytopathic versions. It was previously shown that DNA launched replicons express 5 times more protein compared to traditional pDNA in C2C12 cells (Ljungberg K et al. J Virol. 2007).

Here, the properties of mc and DREP systems were compared and the possibility of applying mc technology in combination with the replicon by generating mc-DREP construct was tested. This could further improve long-term expression levels, provide effective expression without highly efficient delivery in vivo and enable novel regulatory mechanisms.

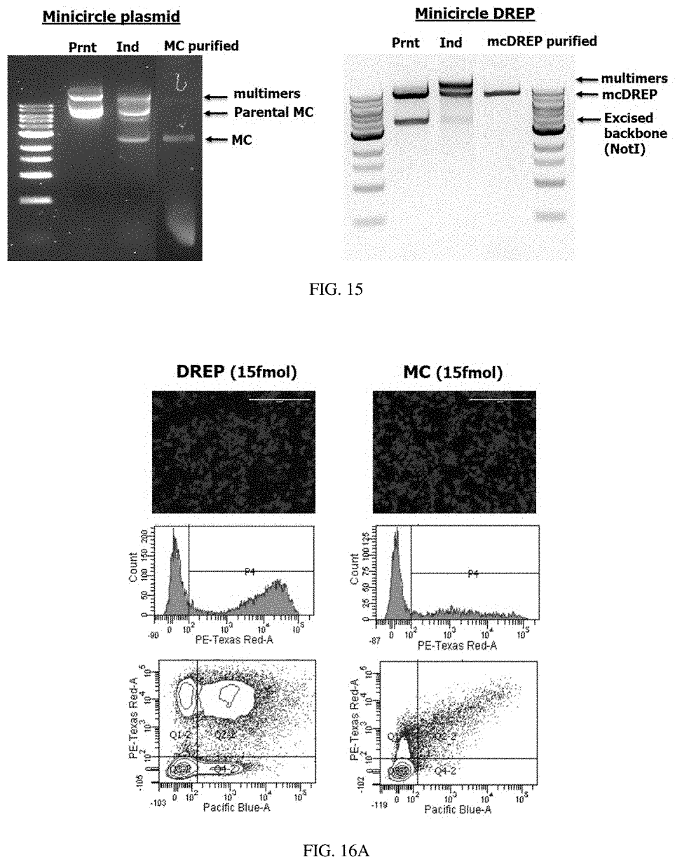

Major limitations in the use of the minicircles are the low yields and contamination with the input minicircle producer plasmid due to a lack of complete recombination between the attB and attP attachment sites. The most efficient system reported to date uses multiple copies (6 or 10) of the chromosomally integrated phiC31 recombinase gene to improve the recombination efficiency (Kay M A, et al. Nat Biotechnol 2010). These approaches still suffer from the low yield, high contamination of the parental plasmid, genomic DNA and multimeric forms, and the need to use expensive and laborious purification procedures. The standard minicircle production procedure was optimized in order to improve the yield and purity of the mc (FIG. 15). The optimization included the following parameters: addition of glucose to the starter culture to inhibit plasmid recombination prior to induction; complete media exchange during the induction; induction at increased OD levels (OD600˜4) and increased Ara concentration (0.1%); mc purification by gel extraction or in vitro enzymatic cleavage (I-SceI+exoV). Mc production was confirmed by PCR test and sequencing. The estimated yield of the purified mc was ˜10 ug/50 ml bacterial culture. Column purification methods (HPLC and size exclusion chromatography) for large scale production for in vivo applications are currently in development.

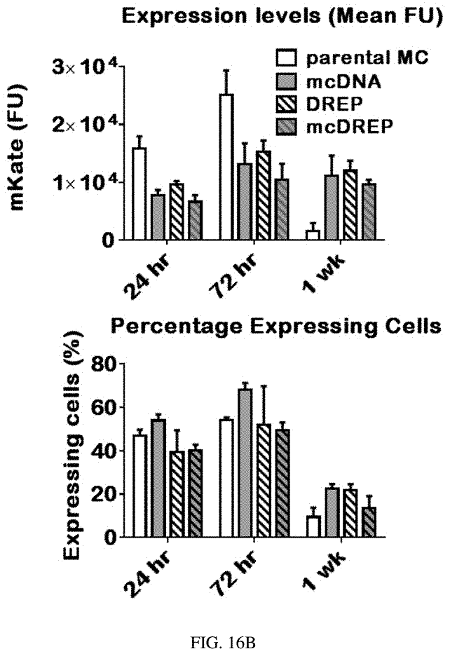

To test the expression levels and possible toxicity of mc and DREP in mammalian cells, HEK293a cells were transiently transfected with mcDNA (parental DNA and excised mc) and mcDREP (parental DREP and excised mcDREP) and followed mKate expression levels during one week of cell growth (FIGS. 16A-16B). EBFP expressing plasmid was used as a transfection control.

Strong levels of mKate expression were observed with all four constructs at 24 hours post-transfection (FIGS. 16A-16B). DREP exhibited a bimodal distribution of positive and negative cells typical for the replicon expression, while mc DNA showed a linear correlation between mKate expression and the amount of transfected plasmid (FIG. 16A). Expression levels peaked at 72 hours post-transfection, with ˜60% positive cells for all the constructs (FIG. 16B). After a week of cell growth there was still a significant level of mKate expression from mc and DREP, while most expression from plasmid DNA had disappeared (FIG. 16B). Similar results were obtained for CHO-K1 cells (not shown).

Thus, mc and DREP yield prolonged and highly increased expression levels. This may have been due to the self-replicating nature of the replicon that leads to their prolonged expression even in the dividing cells in absence of selection, while the plasmid DNA is quickly diluted out of cells.

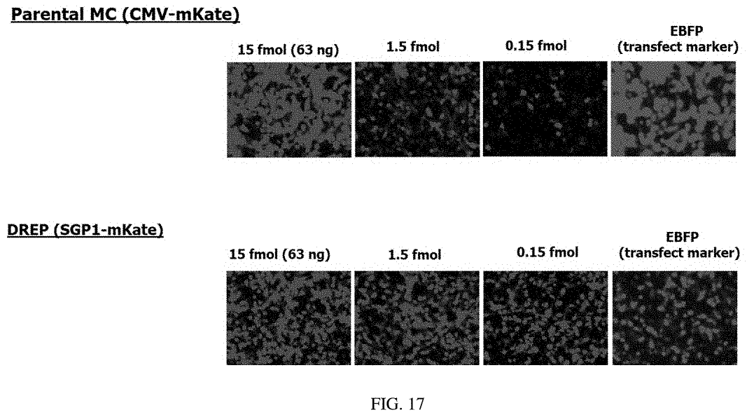

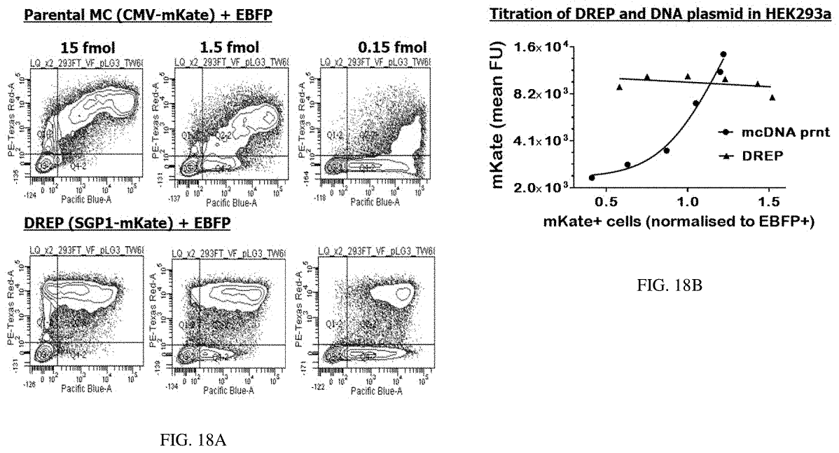

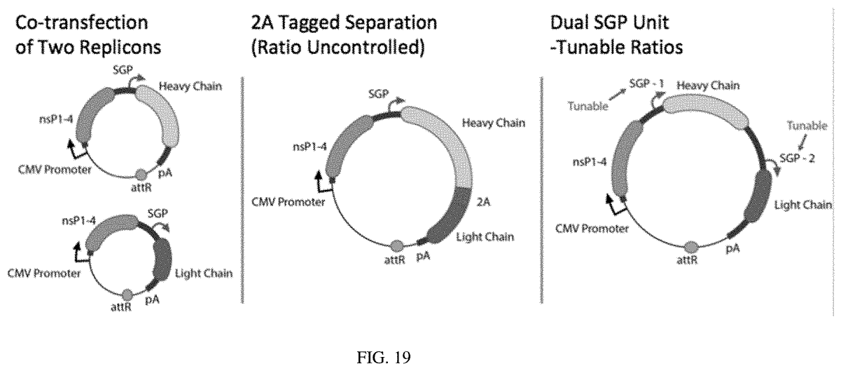

To further investigate the nature of bi-modal DREP expression versus continuous mc expression (FIG. 16A), a plasmid titration experiment was performed in which decreasing amounts of DREP and mc plasmids expressing mKate were co-transfected with a fixed amount of EBFP transfection marker. Fluorescent images of HEK293a transfected cells and FACS analysis of mKate and EBFP levels are shown in FIGS. 17 and 18A-18B, respectively. While there was a concentration dependency of expression levels and the amount of transfected mc plasmid, there was a consistent intensity of DREP expression regardless the amount of transfected DREP. This behavior is unique to replicon since even the lowest amount of transfected DREP is able to launch RNA replicon that will self-amplify and yield high expression levels. Cells that express DNA replicon express at a consistent intensity regardless of the amount of delivered vector. This unique characteristic of DREP is especially important at low amounts of transfected DNA where DREP yields ˜5-fold higher expression levels than mc (FIG. 18A-18B). Similar results were observed in C2C12 cells (not shown). By encoding an amplification competent RNA replicon on DNA plasmids, highly efficient expression were achieved at very low transfection efficiency (which mimics the situation of low transfection efficiency during intramuscular DNA delivery in vivo).

### Example 5. DNA Launched Replicon for Antibody Expression

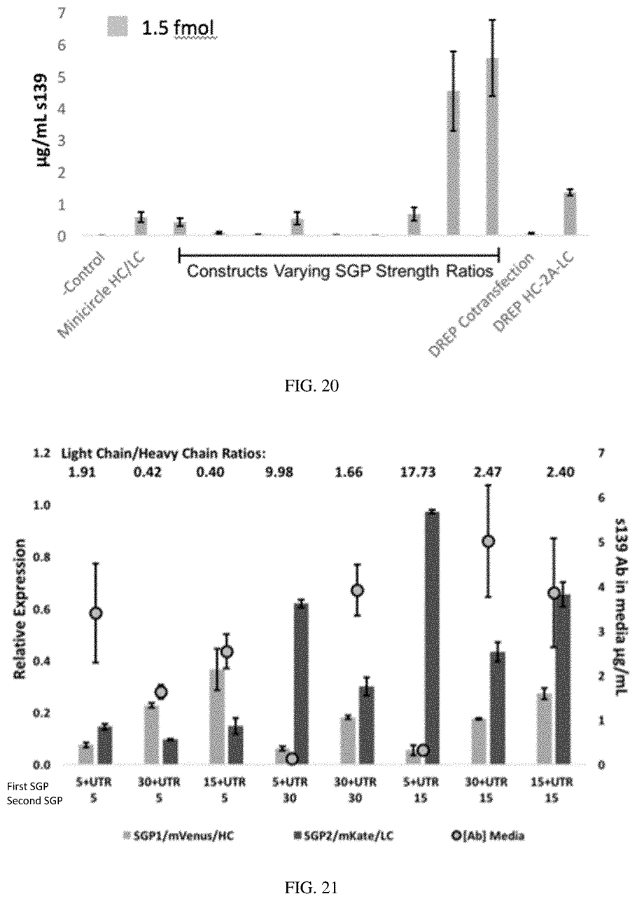

Once a purification strategy for the replicon minicircle constructs was developed, the system was tested as a general platform for the expression of monoclonal antibodies (mAbs). Several strategies were implemented, including co-transfected cassettes each expressing a single chain, a 2A separated heavy chain and light chain, and a construct that employed the RNA toolkit developed earlier in this work. As shown in FIGS. 20-21, the dual unit replicons work exceptionally well and allowed for the expression levels to be modulated by simply tuning the promoter strengths of each individual SGP. Again, under rare delivery conditions the DNA launched replicon was exceptionally capable at producing high titers of antibody in the media.

Using the tools developed on the RNA replicon, the ratios and overall level of mAb heavy chain and light chain can be tuned.

Additionally, the expression levels can be mapped to the ratio and level of expression from each SGP as shown below. When the light-to-heavy ratio is between 1.5 and 2.5 and the light chain is highly expressed (as establish by fluorescent proteins) provide the optimal conditions for mAb production and export into the media (FIG. 21).

### Example 6. DD Tag/Small Molecule Responsive RBP

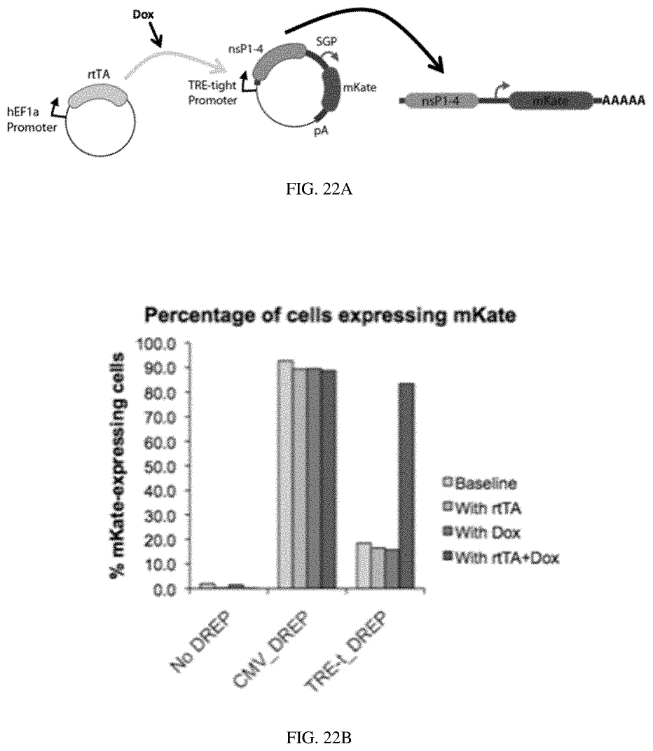

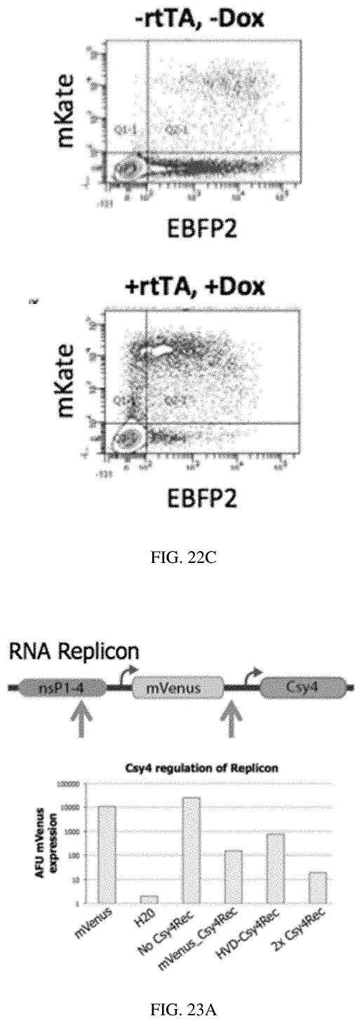

For transcription-level control of DREP expression, an inducible TRE-tight promoter was incorporated upstream of the replicon (FIG. 22A). The activator protein rtTA binds to and induces the promoter only in the presence of Dox. The data indicates that the TRE-tight promoter provides good regulation of DREP, with full expression occurring only in the presence of both rtTA and Dox (FIG. 22B). In the absence of rtTA and Dox, less than 20% of transfected cells expressed mKate (FIG. 22C). However, in each of these “leaky” cells mKate expression reached its maximum. This is due to the self-amplification of the replicon; very few initial RNA transcripts are required to initiate full replicon expression.

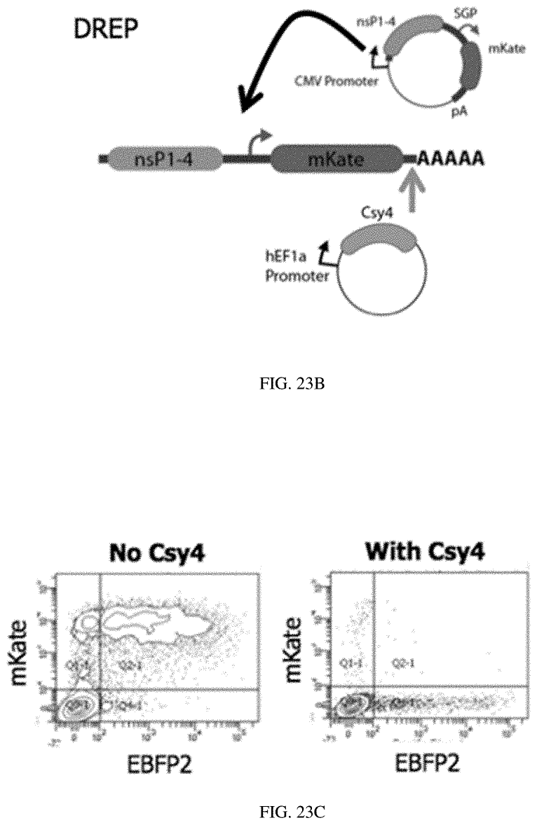

For tighter control of DREP expression, the CRISPR associated RNA endoribonuclease Csy4 was employed. Csy4 binds to and cleaves a specific 28-nt RNA sequence (which does not appear in human or mouse genomes). The Csy4 recognition site can be incorporated into the 3′ UTR of the replicon, resulting in destruction of the full-length replicon and any subgenomic mRNAs that share the 3′ UTR. A Csy4 recognition site can also be placed into the hypervariable domain (HVD) near the 3′ end of nsP3, resulting in destruction of the full-length replicon but not the subgenomic mRNAs. For the RNA replicon, both the HVD and 3′ UTR loci were tested for the Csy4 recognition site with a Csy4 expressed from an SGP, and observed up to 100-fold repression of the replicon (FIG. 23A). Next, the Csy4 recognition site was introduced into the 3′ UTR of DREP. Csy4 was expressed from a separate plasmid under control of a constitutive promoter (FIG. 23B). Csy4 was able to nearly entirely eliminate replicon expression (FIG. 23C).

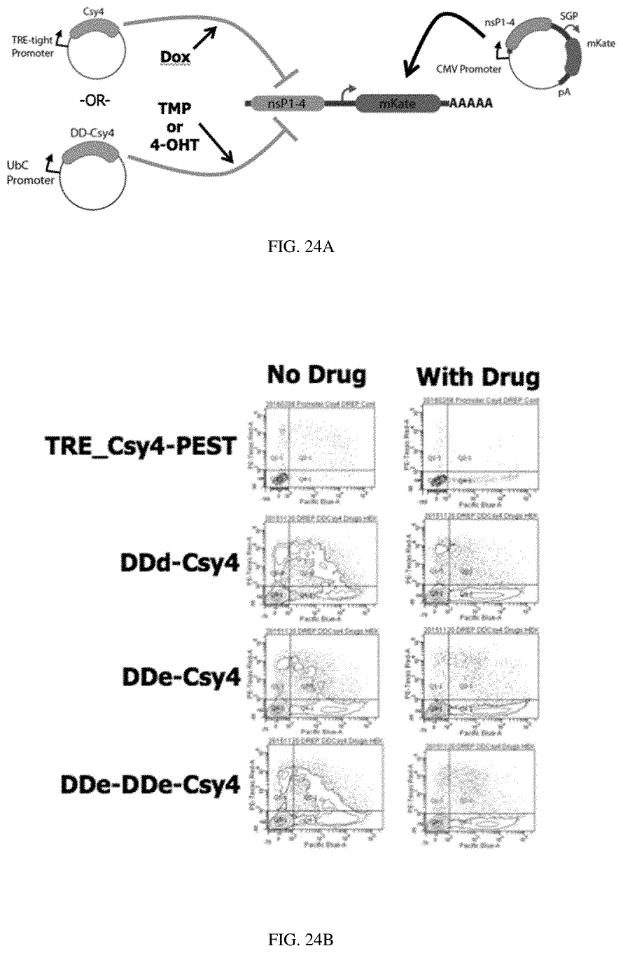

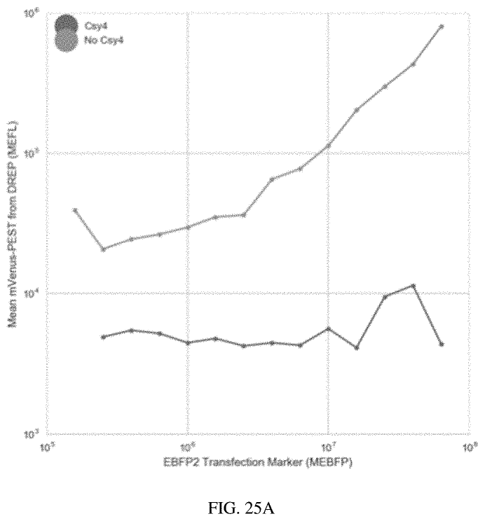

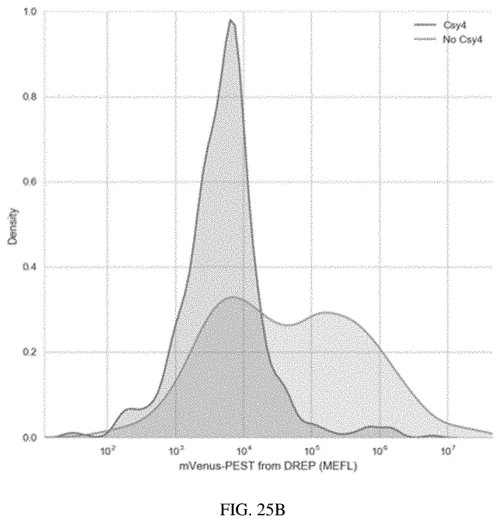

In order to provide external control over the “on” or “off” states of the replicon, small-molecule control mechanisms were added to Csy4. These mechanisms included expressing Csy4 under control of rtTA and Dox, or adding degradation domains (DD) to Csy4, which would be stabilized by the presence of TMP or 4-OHT (FIG. 24A). In each case, the DREP would be active in the absence of drug and repressed by Csy4 in the presence of drug. Several of the topologies tested allowed for small-molecule control of DREP (FIG. 24B). Further, the control of Csy4 DREP bearing Csy4 target sites were validated, shown in FIGS. 25A-B, that the presence of Csy4 inhibited the expression of DREP-mVenus.

### Example 7: Validation of DREP in Engineered HSV-1 Genome

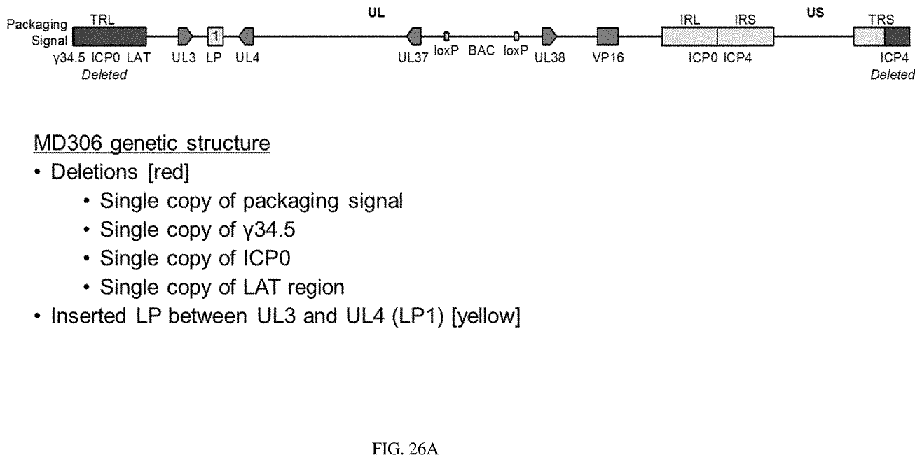

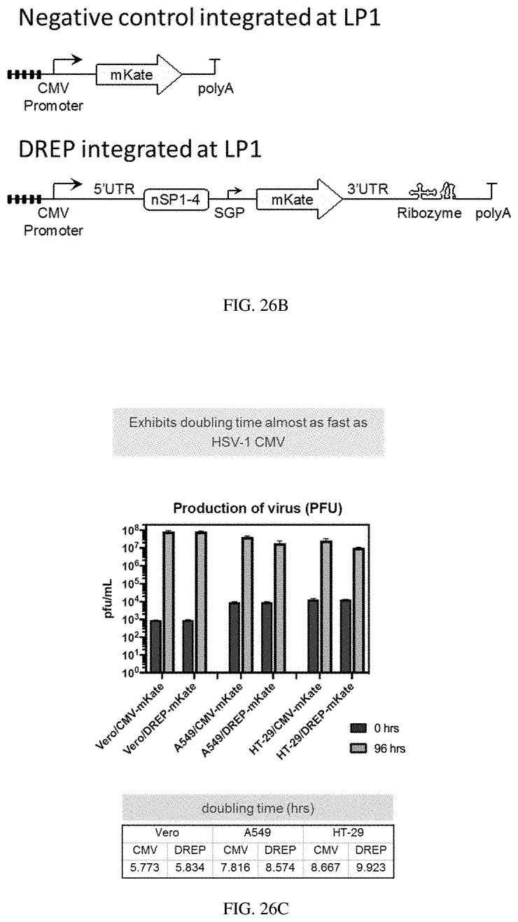

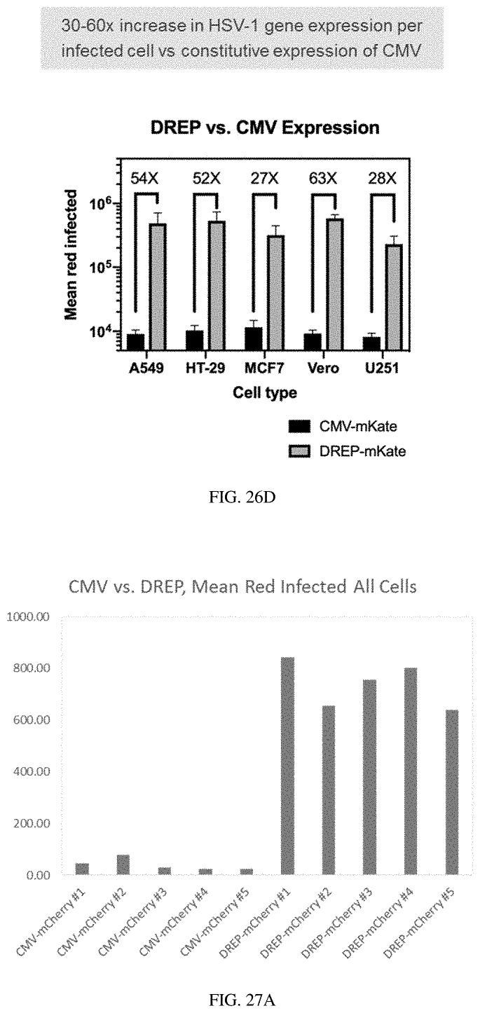

To test DREP in an engineered HSV-1 genome (MD306), a HSV-1 genome was designed to include a landing pad (LP1) such that the DREP can be integrated at LP1 location. As shown in FIG. 26A, the engineered HSV-1 genome includes a single copy of packaging signal, γ34.5, ICP0, with an LAT region deletion from HSV-1 Strain KOS. The landing pad (LP1) was placed in the genome between UL3 and UL4. FIG. 26B shows a negative control construct (CMV-mKate) and a DREP construct (DREP-mKate) to be integrated at LP1. The DREP-mKate construct includes a CMV promoter, 5′UTR, nSP1-4, subgenomic promoter (SGP), mKate, 3′UTR, ribozyme, and polyA.

First, the effect of DREP on the replication rate of HSV-1 was determined by counting plaque forming units (pfu) in Vero, A549, and HT-29 cells at 0 and 96 hrs post infection. In order to integrate the DREP into MD306 viral genome and package the HSV-1 virus, DREP-mKate DNA were respectively co-transfected with CMV-ICP4-2A-ICP27-2A-VP16, pCAG-Cre, and pCAG-Flp into U2OS::pBjh5928 packaging cell line with 1 ug/mL dox. CMV-mkate was integrated and packaged using the same method as negative control. Viruses were harvested and titrated in the packaging cell line by plaque assay. 24 hours before infection, ˜5×10⁵ cells of Vero, A549, and HT-29 were seeded in 6 well plates. Then cells were infected with each virus at MOI 0.001 for Vero and 0.01 for A549 and HT-29 in triplicate. The plates for ‘0 hour’ were frozen immediately at −80° C. and the plates for ‘96 hours’ were frozen 96 hours post infection. Viruses were harvested and titrated in the packaging cell line. The doubling time for each virus was calculated using the pfu counted at two time points. The pfu of both virus were similar in Vero and slightly lower in A549 and HT-29 after 96 hours. The calculated doubling time reflected similar growth rates (FIG. 26C). To determine expression level of mKate, 1×10⁵ cells of Vero, A549, HT-29, MCF7, and U251 were seeded in 24 well plates 24 hours before infection. Then cells were infected with each virus at MOI 0.001 for Vero, MCF7, and U251 and 0.01 for A549 and HT-29 in triplicate. The infected cells were prepared and data was collected by flow cytometry 24, 48, 72, and 96 hours post infection. The mean of mKate expression level of infected cells was calculated for each time point and averaged to calculate overall mean of mKate expression in each cell line. Expression level of mKate of DREP-mKate was about 30-60 fold higher than that of CMV-mKate (FIG. 26D).

For in vivo validation of the HSV1-DREP constructs, different negative control constructs and DREP constructs were used. In this experiment, mCherry replaced mKate in the constructs shown in FIG. 26B. The HSV1-CMV-mCherry and HSV1-DREP-mCherry were packaged using the same method described above. 1×10⁶ 4 T1 breast cancer cells were injected in the mammary fat pad of female BALB/C mice. 7 days later, 5×10⁶ pfu of HSV1-CMV-mCherry or HSV1-DREP-mCherry were injected into the tumors of five mice per group. 24 hours later, tumors were harvested and mCherry intensity was measured by FACS. On average, mCherry in vivo expression levels from DREP-mCherry were 18 fold higher than from CMV-mCherry (FIGS. 27A-27B).

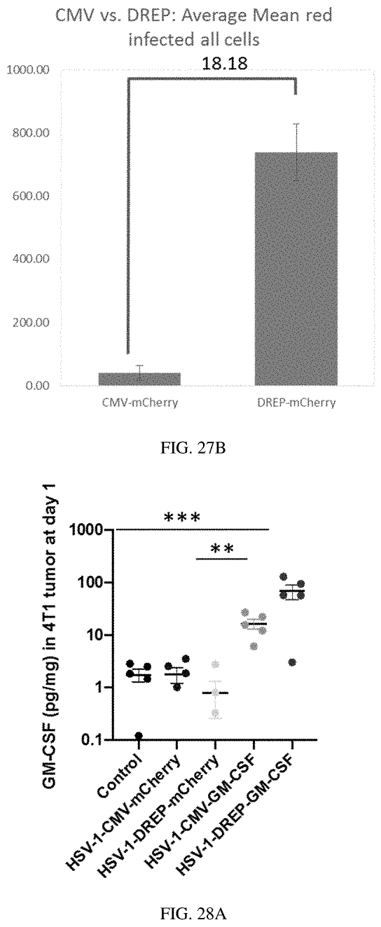

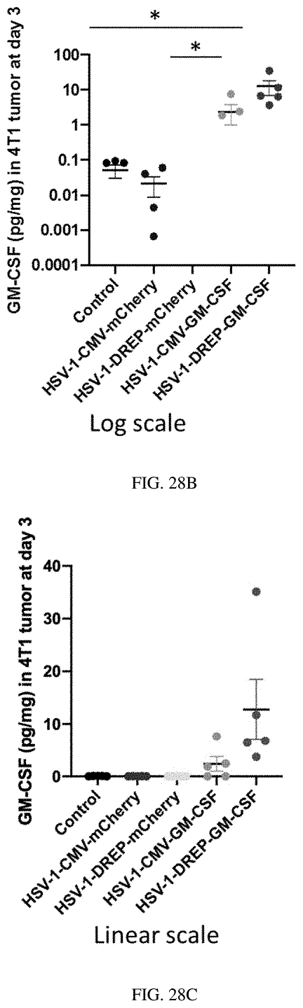

DREP expression of cytokines have also been validated in vivo. CMV-GM-CSF and DREP-GM-CSF were constructed by replacing mKate in the constructs shown in FIG. 26B with GM-CSF. PBS containing 10% glycerol, CMV-mCherry, and DREP-mCherry were used as controls. The HSV1 viruses carrying each construct were generated using the method described above. 1×10⁶ 4 T1 breast cancer cells were injected to the right flank of the female BALB/C mice. When the tumor area reached 50 mm2 (˜7 days), the mice were intratumorally injected with 1×10⁷ pfu of CMV-mCherry, DREP-mCherry, CMV-GM-CSF, or DREP-GM-CSF (N=5 in each group) respectively. PBS+10% glycerol was injected to the control group (N=5). 24 and 72 hours later, the tumors were necropsied and homogenized for measuring level of GM-CSF by ELISA. On average, GM-CSF in vivo expression levels from DREP-GM-CSF were 4 fold higher than from CMV-GM-CSF after 1 day post injection (FIG. 28A) and 5 fold higher after 3 days (FIG. 28B in log scale and FIG. 28C in linear scale) post injection. Note that no data points were graphed for DREP-mCherry at day 3, because GM-CSF was not detected in that group and data was graphed in log scale.

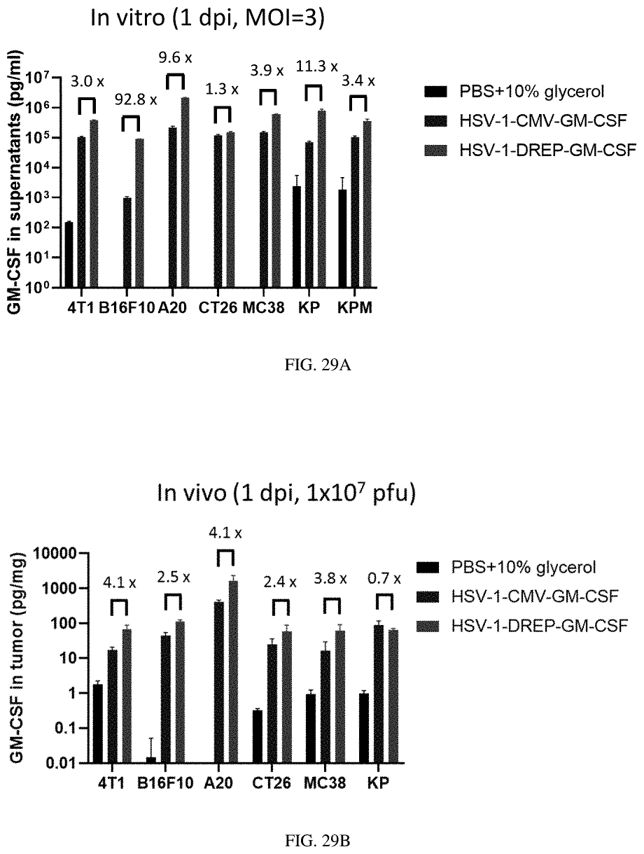

For in vitro and in vivo validation of cytokine expression level from DREP in additional tumor models, 4T1, B16F10, A20, CT26, MC38, KP (Kras/P53), and KPM cells were tested using HSV1-CMV-GM-CSF and HSV1-DREP-GM-CSF. For in vitro experiments, 4T1, B16F10, A20, CT26, MC38, KP, and KPM cells were seeded 24 hours before infection in 96 well plates, and HSV1-CMV-GM-CSF and HSV1-DREP-GM-CSF were added to each well at MOI of 3. As a negative control, PBS+10% glycerol was added. 24 hours later, GM-CSF expression was measured by ELISA from supernatant of infected wells. 4T1, A20, CT26, MC38, KP, and KPM showed reasonable infection rates and DREP-GM-CSF resulted in higher GM-CSF expression levels than CMV-GM-CSF. B16F10 showed 92.8 fold increase but had a poor infection rate (FIG. 29A). For in vivo experiments, 1×10⁶ cells of B16F10, MC38, KP (Kras/P53) cells were subcutaneously inoculated to the right flank of the C57BL/6J mice. Similarly, 1×10⁶ cells of 4T1, CT26, or 2×10⁶ cells of A20 cells were injected to the right flank of the Balb/c mice. The KPM mouse model was excluded from the in vivo experiments. When the tumor area reached 50 mm2 (˜7-9 days), the mice were intratumorally injected with 1×10⁷ pfu of HSV1-CMV-GM-CSF or HSV1-DREP-GM-CSF (N=5). PBS+10% glycerol was injected to the negative control group (N=5). A day post injection of viruses, the tumor were necropsied and homogenized for measuring level of GM-CSF by ELISA. DREP-GM-CSF resulted in higher GM-CSF expression than CMV-GM-CSF in all tumor models except for KP (FIG. 29B).

### OTHER EMBODIMENTS

1. An expression system comprising:
(i) a promoter operably linked to a nucleotide sequence encoding one or more viral non-structural proteins, and
(ii) a subgenomic viral promoter operably linked to a nucleotide sequence encoding an output molecule.
   2. The expression system of paragraph 1, wherein the promoter is a constitutive promoter.
   3. The expression system of paragraph 1 or paragraph 2, wherein the promoter is a CMV promoter or a variant thereof.
   4. The expression system of paragraph 1, wherein the promoter is an inducible promoter.
   5. The expression system of paragraph 4, wherein the inducible promoter is activated or repressed by a signal produced from a cell classifier.
   6. The expression system of any one of paragraphs 1-5, wherein (ii) further comprises a nucleotide sequence encoding a 3′ untranslated region (3′UTR) downstream of the nucleotide sequence encoding the output molecule.
   7. The expression system of paragraph 6, wherein (ii) further comprises a poly-adenylation (polyA) signal sequence downstream of the 3′UTR.
   8. The expression system of paragraph 7, wherein the polyA signal sequence of (ii) comprises a transcriptional terminator.
   9. The expression system of paragraph 8, wherein the transcriptional terminator is selected from BGH_TT, antigenomic-BGH_TT, rb_glob_TT, and antigenomic_HD-SV40_TT.
   10. The expression system of any one of paragraphs 1-9, wherein (ii) further comprises a nucleotide sequence encoding one or more cleavage sites for an endoribonuclease.
   11. The expression system of paragraph 10, further comprising
(iii) a promoter operably linked to a nucleotide sequence encoding an endoribonuclease that cleaves at the one or more cleavage sites.
   12. The expression system of paragraph 10 or paragraph 11, wherein the endoribonuclease is selected from Csy4, Cse3, Cas6, Csy13, CasE, and variants thereof.
   13. The expression system of paragraph 11 or paragraph 12, wherein the promoter in (iii) is an inducible promoter.
   14. The expression system of paragraph 13, wherein the inducible promoter is regulated by a small molecule.
   15. The expression system of paragraph 14, wherein the small molecule is doxycycline or abscisic acid.
   16. The expression system of any one of paragraphs 11-15, wherein the nucleotide sequence encoding the endoribonuclease is operably linked to a nucleotide sequence encoding a degradation signal.
   17. The expression system of paragraph 16, wherein the degradation signal is selected from: PEST, a destabilization domain from E. coli dihydrofolate reductase (ecDHFR), or a destabilization domain derived from human FKBP protein.
   18. The expression system of paragraph 17, wherein degradation of Csy4 mediated by the degradation signal is inhibited in the presence of TMP or 4-OHT.
   19. The expression system of any one of paragraphs 1-18, wherein the one or more viral proteins are selected from: NSP 1-4.
   20. The expression system of any one of paragraphs 1-19, wherein the expression system is one or more engineered viral genomes.
   21. The expression system of paragraph 20, wherein the viral genome is the genome of an oncolytic virus.
   22. The expression system of paragraph 21, wherein the oncolytic virus is selected from the group consisting of: alphaviruses, adenoviruses, reoviruses, measles virus, herpes simplex virus, Newcastle disease virus and vaccinia virus.
   23. The expression system of paragraph 22, wherein the oncolytic virus is herpes simplex virus 1 (HSV-1).
   24. The expression system of any one of paragraphs 1-23, wherein the expression system is one or more Minicircle DNA molecules.
   25. The expression system of any one of paragraphs 1-24, wherein the output molecule is a nucleic acid, a detectable molecule, or a therapeutic molecule.
   26. The expression system of paragraph 25, wherein the nucleic acid is a DNA or RNA.
   27. The expression system of paragraph 25, wherein the therapeutic molecule is a therapeutic protein.
   28. The expression system of paragraph 27, wherein the therapeutic protein is an antigen, an antibody, an enzyme, a regulatory protein, an immunomodulator, a cytokine, or a chemokine.
   29. The expression system of paragraph 28, wherein the therapeutic protein is a cytokine, and wherein the cytokine is GM-CSF, IL-4, IL6, IL10, IL11, IL13, IL-1ra, TGF-β, IFNg, IL15, CXCL10, CCL4, CD40L, secreted CD40L, IL12, MLKL and variants thereof, scIL-27, secreted HMGB1, or HMGB1.
   30. The expression system of any one of paragraphs 1-28, further comprising a ribozyme located between the 3′UTR and the polyA signal sequence.
   31. A viral particle comprising the expression system of any one of paragraphs 1-30, or the antibody expression system of paragraphs 50-53.
   32. A cell comprising the expression system of any one of paragraphs 1-30, or the antibody expression system of paragraphs 50-53.
   33. The cell of paragraph 32, wherein the cell is a diseased cell.
   34. The cell of paragraph 33, wherein the diseased cell is a cancer cell.
   35. The cell of paragraph 32, wherein the cell is a healthy cell.
   36. The cell of paragraph 35, wherein the cell is an immune cell.
   37. A method of expressing an output molecule, comprising delivering the expression system of any one of paragraphs 1-30, the antibody expression system of paragraphs 50-53, or the viral particle of paragraph 31 to a cell and culturing the cell under conditions that allow expression of the output molecule.
   38. The method of paragraph 37, wherein the promoter operably linked to the nucleotide sequence encoding one or more viral non-structural proteins is an inducible promoter, and the method further comprises providing an inducer that activates the promoter.
   39. The method of paragraph 37 or paragraph 38, wherein the cell is in vitro. 40. The method of paragraph 37 or paragraph 38, wherein the cell is ex vivo.
   41. The method of paragraph 37 or paragraph 38, wherein the cell is in vivo.
   42. The method of any one of paragraphs 37-41, wherein the cell is a diseased cell, a healthy cell, or an immune cell.
   43. The method of paragraph 42, wherein the diseased cell is a cancer cell.
   44. The method of paragraph 42, wherein the cell is a healthy cell.
   45. The method of paragraph 42, wherein the cell is an immune cell.
   46. A method of treating a disease, comprising administering to a subject in need thereof an effective amount of the expression system of any one of paragraphs 1-30, the antibody expression system of paragraphs 50-53, or the viral particle of paragraph 31.
   47. The method of paragraph 46, wherein the disease is cancer.
   48. A composition comprising the expression system of any one of paragraphs 1-30, the antibody expression system of paragraphs 50-53, or the viral particle of paragraph 31.
   49. The composition of paragraph 48, further comprising a pharmaceutically acceptable carrier.
   50. An antibody expression system comprising a promoter operably linked to a nucleic acid comprising a nucleotide sequence encoding one or more viral non-structural proteins, and comprising:
(a) a first subgenomic viral promoter operably linked to a nucleotide sequence encoding a first antibody; and
(b) a second subgenomic viral promoter operably linked to a nucleotide sequence encoding a second antibody.
   51. The antibody expression system of paragraph 50, wherein the first antibody is a single chain variable fragment (scFv) and/or the second antibody is a single chain variable fragment (scFv).
   52. The antibody expression system of paragraph 50, wherein the first antibody is a single variable domain, such as a VH or VHH, and/or the second antibody is single variable domain, such as a VH or VHH.
   53. The antibody expression system of any one of paragraph 50-52, wherein the first antibody and the second antibody are different.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An antibody expression system comprising a promoter operably linked to a nucleic acid comprising a nucleotide sequence encoding one or more viral non-structural proteins, and comprising:
(a) a first viral subgenomic promoter operably linked to a nucleotide sequence encoding an immunoglobulin heavy chain; and
(b) a second viral subgenomic promoter operably linked to a nucleotide sequence encoding an immunoglobulin light chain.

2. An antibody expression system comprising:
(a) a promoter operably linked to a first nucleic acid comprising a nucleotide sequence encoding one or more viral non-structural proteins and a first viral subgenomic promoter operably linked to a nucleotide sequence encoding an immunoglobulin heavy chain; and
(b) a promoter operably linked to a second nucleic acid comprising a nucleotide sequence encoding one or more viral non-structural proteins and a second viral subgenomic promoter operably linked to a nucleotide sequence encoding an immunoglobulin light chain.

3.-8. (canceled)

9. The antibody expression system of claim 2, wherein (a) further comprises a nucleotide sequence encoding a 3′ untranslated region (3′UTR) downstream of the nucleotide sequence encoding the immunoglobulin heavy chain, and/or (b) further comprises a nucleotide sequence encoding a 3′ untranslated region (3′UTR) downstream of the nucleotide sequence encoding the immunoglobulin light chain.

10. (canceled)

11. The antibody expression system of claim 9, wherein (a) further comprises a poly-adenylation (polyA) signal sequence downstream of the 3′UTR, and/or (b) further comprises a poly-adenylation (polyA) signal sequence downstream of the 3′UTR.

12.-15. (canceled)

16. The antibody expression system of claim 11, wherein (a) further comprises a ribozyme located between the 3′UTR and the polyA signal, and/or (b) further comprises a ribozyme located between the 3′UTR and the polyA signal

17. The antibody expression system of claim 2, wherein the first viral subgenomic promoter is different from the second viral subgenomic promoter.

18. The antibody expression system of claim 2, wherein the first viral subgenomic promoter and the second viral subgenomic promoter lead to different expression levels of the heavy chain and the light chain.

19.-20. (canceled)

21. The antibody expression system of claim 2, wherein (a) and/or (b) further comprises a nucleotide sequence encoding one or more cleavage sites for an endoribonuclease.

22. The antibody expression system of claim 21, further comprising a promoter operably linked to a nucleotide sequence encoding an endoribonuclease that cleaves at the one or more cleavage sites, wherein the endoribonuclease is Csy4, Cse3, Cas6, Csy13, or CasE.

23.-26. (canceled)

27. The antibody expression system of claim 22, wherein the nucleotide sequence encoding the endoribonuclease is operably linked to a nucleotide sequence encoding a degradation signal, optionally wherein the degradation signal is a PEST, a destabilization domain from E. coli dihydrofolate reductase (ecDHFR), or a destabilization domain derived from human FKBP protein.

28.-30. (canceled)

31. The antibody expression system of claim 2, wherein the immunoglobulin is an immunoglobulin G (IgG), an immunoglobulin M (IgM), an immunoglobulin A (IgA), an immunoglobulin D (IgD) or an immunoglobulin E (IgE).

32. The antibody expression system of claim 2, wherein the immunoglobulin is an immune checkpoint inhibitor, optionally wherein the immune checkpoint inhibitor is selected from: anti-CTLA4, anti-PD1, and anti-PD-L1.

33.-34. (canceled)

35. The antibody expression system of claim 2, wherein the antibody expression system is one or more engineered viral genomes.

36. The antibody expression system of claim 35, wherein the viral genome is the genome of an oncolytic virus, optionally wherein the oncolytic virus is selected from the group consisting of: alphaviruses, adenoviruses, reoviruses, measles virus, herpes simplex virus, Newcastle disease virus and vaccinia virus, optionally wherein the oncolytic virus is herpes simplex virus 1 (HSV-1).

37.-39. (canceled)

40. A viral particle comprising the antibody expression system of claim 2.

41. A cell comprising the antibody expression system of claim 2.

42.-45. (canceled)

46. A method of expressing an immunoglobulin, comprising delivering the antibody expression system of claim 2 to a cell and culturing the cell under conditions that allow expression of the light chain and the heavy chain.

47.-54. (canceled)

55. A method of treating a disease, comprising administering to a subject in need thereof an effective amount of the antibody expression system of claim 2.

56.-58. (canceled)

59. A composition comprising the antibody expression system of claim 2.

60. (canceled)

61. A method of producing an antibody expression system, comprising:
(i) providing a plurality of genetic elements comprising a plurality of viral subgenomic promoters, a nucleotide sequence encoding an immunoglobulin heavy chain, a nucleotide sequence encoding an immunoglobulin light chain, and optionally a nucleotide sequence encoding a 3′ untranslated region (3′UTR), wherein each genetic element is flanked at the 3′ end and the 5′ end by a recognition and cleavage site for a first type IIS restriction endonuclease, and wherein the recognition and cleavage site is engineered to allow directional assembly of the plurality genetic elements;
(ii) assembling a first transcriptional unit comprising, in order from 5′ to 3′, a first viral subgenomic promoter, the nucleotide sequence encoding the immunoglobulin heavy chain, and optionally the nucleotide sequence encoding the 3′UTR, by combining the genetic elements with:
(a) the first type IIS restriction endonuclease;
(b) a ligase; and
(c) a first destination vector comprising a pair of the recognition and cleavage sites for the first type IIS restriction endonuclease and a pair of the recognition and cleavage sites for a second type IIS restriction endonuclease, wherein the pair of recognition and cleavage sites for the second type IIS restriction endonuclease enclose the pair of recognition and cleavage sites for the first type IIS restriction endonuclease, and wherein the two pairs of recognition and cleavage sites are positioned in inverse orientation relative to each other;
wherein the contacting is carried out under conditions that allow the cleavage at the recognition and cleavage sites for the first type IIS restriction endonuclease and the ligation of resulting fragments in a directional manner;
(iii) assembling a second transcriptional unit comprising, in order from 5′ to 3′, a second viral subgenomic promoter, the nucleotide sequence encoding the immunoglobulin light chain, and optionally the nucleotide sequence encoding the 3′UTR, by contacting the genetic elements with:
(a) the first type IIS restriction endonuclease;
(b) a ligase; and
(c) a first destination vector comprising a pair of the recognition and cleavage sites for the first type IIS restriction endonuclease and a pair of the recognition and cleavage sites for a second type IIS restriction endonuclease, wherein the pair of recognition and cleavage sites for the second type IIS restriction endonuclease enclose the pair of recognition and cleavage sites for the first type IIS restriction endonuclease, and wherein the two pairs of recognition and cleavage sites are positioned in inverse orientation relative to each other;
wherein the contacting is carried out under conditions that allow the cleavage at the recognition and cleavage sites for the first type IIS restriction endonuclease and the ligation of resulting fragments in a directional matter;
(iv) assembling the antibody expression system by combining the first transcriptional unit obtained in (ii) and the second transcriptional unit obtained in (iii) with:
(a) the second type IIS restriction endonuclease;
(b) a ligase; and
(c) a second destination vector comprising a promoter operably linked to a nucleotide sequence encoding one or more viral non-structural proteins, and a pair of the recognition and cleavage sites for the second type IIS restriction endonuclease,
wherein the contacting is carried out under conditions that allow the cleavage at the recognition and cleavage sites for the second type IIS restriction endonuclease and the ligation of resulting fragments in a directional manner.

62.-64. (canceled)


SEQUENCE LISTING (25 sequences)

SEQ ID NO: 1 - DNA, 235 bp, Artificial Sequence
  Synthetic polynucleotide
cagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggc

SEQ ID NO: 2 - DNA, 541 bp, Artificial Sequence
  Synthetic polynucleotide
tgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctg

SEQ ID NO: 3 - DNA, 201 bp, Artificial Sequence
  Synthetic polynucleotide
agcggccgcctgcagcttaagaccggtaagctaagctacgcgtgctagcgggcccgttaacttgtttattgcagcttataatggttacaaataaagcaatagcatcacaaatttcacaaataaagcatttttttcactgcattctagttgtggtttgtccaaactcatcaatgtatcttatcatgtctggatcttaattaa

SEQ ID NO: 4 - DNA, 1605 bp, Venezuelan equine encephalitis virus
atggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggcc

SEQ ID NO: 5 - DNA, 2382 bp, Venezuelan equine encephalitis virus
ggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggctgttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgt

SEQ ID NO: 6 - DNA, 1650 bp, Venezuelan equine encephalitis virus
gcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaa

SEQ ID NO: 7 - DNA, 1821 bp, Venezuelan equine encephalitis virus
tacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggc

SEQ ID NO: 8 - protein, 535 aa, Venezuelan equine encephalitis virus
MEKVHVDIEEDSPFLRALQRSFPQFEVEAKQVTDNDHANARAFSHLASKLIETEVDPSDTILDIGSAPARRMYSKHKYHCICPMRCAEDPDRLYKYATKLKKNCKEITDKELDKKMKELAAVMSDPDLETETMCLHDDESCRYEGQVAVYQDVYAVDGPTSLYHQANKGVRVAYWIGFDTTPFMFKNLAGAYPSYSTNWADETVLTARNIGLCSSDVMERSRRGMSILRKKYLKPSNNVLFSVGSTIYHEKRDLLRSWHLPSVFHLRGKQNYTCRCETIVSCDGYVVKRIAISPGLYGKPSGYAATMHREGFLCCKVTDTLNGERVSFPVCTYVPATLCDQMTGILATDVSADDAQKLLVGLNQRIVVNGRTQRNTNTMKNYLLPVVAQAFARWAKEYKEDQEDERPLGLRDRQLVMGCCWAFRRHKITSIYKRPDTQTIIKVNSDFHSFVLPRIGSNTLEIGLRTRIRKMLEEHKEPSPLITAEDVQEAKCAADEAKEVREAEELRAALPPLAADVEEPTLEADVDLMLQEAGA

SEQ ID NO: 9 - protein, 794 aa, Venezuelan equine encephalitis virus
GSVETPRGLIKVTSYDGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKKMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKMVDWLSDRPEATFRARLDLGIPGDVPKYDIIFVNVRTPYKYHHYQQCEDHAIKLSMLTKKACLHLNPGGTCVSIGYGYADRASESIIGAIARLFKFSRVCKPKSSLEETEVLFVFIGYDRKARTHNPYKLSSTLTNIYTGSRLHEAGC

SEQ ID NO: 10 - protein, 550 aa, Venezuelan equine encephalitis virus
APSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVDETPEPSAENQSTEGTPEQPPLITEDETRTRTPEPIIIEEEEEDSISLLSDGPTHQVLQVEADIHGPPSVSSSSWSIPHASDFDVDSLSILDTLEGASVTSGATSAETNSYFAKSMEFLARPVPAPRTVFRNPPHPAPRTRTPSLAPSRACSRTSLVSTPPGVNRVITREELEALTPSRTPSRSVSRTSLVSNPPGVNRVITREEFEAFVAQQQ

SEQ ID NO: 11 - protein, 607 aa, Venezuelan equine encephalitis virus
YIFSSDTGQGHLQQKSVRQTVLSEVVLERTELEISYAPRLDQEKEELLRKKLQLNPTPANRSRYQSRRVENMKAITARRILQGLGHYLKAEGKVECYRTLHPVPLYSSSVNRAFSSPKVAVEACNAMLKENFPTVASYCIIPEYDAYLDMVDGASCCLDTASFCPAKLRSFPKKHSYLEPTIRSAVPSAIQNTLQNVLAAATKRNCNVTQMRELPVLDSAAFNVECFKKYACNNEYWETFKENPIRLTEENVVNYITKLKGPKAAALFAKTHNLNMLQDIPMDRFVMDLKRDVKVTPGTKHTEERPKVQVIQAADPLATADLCGIHRELVRRLNAVLLPNIHTLFDMSAEDFDAIIAEHFQPGDCVLETDIASFDKSEDDAMALTALMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAVDDEHDDDRRRALHEESTRWNRVGILPELCKAVESRYETVGTSIIVMAMTTLASSVKSFSYLRGAPITLYG

SEQ ID NO: 12 - DNA, 30 bp, Artificial Sequence
  Synthetic polynucleotide
atggactacgacatagtctagtccgccaag

SEQ ID NO: 13 - DNA, 15 bp, Artificial Sequence
  Synthetic polynucleotide
atggactacgacata

SEQ ID NO: 14 - protein, 264 aa, Pyrococcus furiosus
MRFLIRLVPEDKDRAFKVPYNHQYYLQGLIYNAIKSSNPKLATYLHEVKGPKLFTYSLFMAEKREHPKGLPYFLGYKKGFFYFSTCVPEIAEALVNGLLMNPEVRLWDERFYLHEIKVLREPKKFNGSTFVTLSPIAVTVVRKGKSYDVPPMEKEFYSIIKDDLQDKYVMAYGDKPPSEFEMEVLIAKPKRFRIKPGIYQTAWHLVFRAYGNDDLLKVGYEVGFGEKNSLGFGMVKVEGNKTTKEAEEQEKITFNSREELKTGV

SEQ ID NO: 15 - protein, 187 aa, Pseudomonas aeruginosa
MDHYLDIRLRPDPEFPPAQLMSVLFGKLHQALVAQGGDRIGVSFPDLDESRSRLGERLRIHASADDLRALLARPWLEGLRDHLQFGEPAVVPHPTPYRQVSRVQAKSNPERLRRRLMRRHDLSEEEARKRIPDTVARALDLPFVTLRSQSTGQHFRLFIRHGPLQVTAEEGGFTCYGLSKGGFVPWF

SEQ ID NO: 16 - protein, 199 aa, Escherichia coli
MYLSKIIIARAWSRDLYQLHQELWHLFPNRPDAARDFLFHVEKRNTPEGCHVLLQSAQMPVSTAVATVIKTKQVEFQLQVGVPLYFRLRANPIKTILDNQKRLDSKGNIKRCRVPLIKEAEQIAWLQRKLGNAARVEDVHPISERPQYFSGEGKNGKIQTVCFEGVLTINDAPALIDLLQQGIGPAKSMGCGLLSLAPL

SEQ ID NO: 17 - protein, 211 aa, Thermus thermophilus
MWLTKLVLNPASRAARRDLANPYEMHRTLSKAVSRALEEGRERLLWRLEPARGLEPPVVLVQTLTEPDWSVLDEGYAQVFPPKPFHPALKPGQRLRFRLRANPAKRLAATGKRVALKTPAEKVAWLERRLEEGGFRLLEGERGPWVQILQDTFLEVRRKKDGEEAGKLLQVQAVLFEGRLEVVDPERALATLRRGVGPGKALGLGLLSVAP

SEQ ID NO: 18 - DNA, 795 bp, Pyrococcus furiosus
atgcgcttcctcattcgtctcgtgcctgaggataaggatcgggcctttaaagtgccatataaccatcagtattacctgcagggcctcatctataatgccatcaaatcctccaatccgaagctggccacctacctgcatgaggtgaagggtcccaaactgttcacctacagcctgtttatggccgaaaaacgcgaacaccctaaggggctgccttactttttggggtacaagaagggcttcttctacttttctacctgcgtgccggagatcgctgaagcactggtcaacggactcctgatgaatccagaggtgcgcctgtgggacgaacgcttctacctgcacgaaattaaggttttgagagagcctaagaagttcaacggctctaccttcgtcaccctgtctccgattgctgtgactgtcgtgaggaagggtaagagttatgatgtcccccctatggagaaggagttttacagtatcatcaaagacgacctgcaagataagtatgtgatggcctacggcgacaagcccccatccgaattcgagatggaggtgctgattgctaagccgaaacggtttcgtattaagcctggcatctatcagacagcctggcacctggtttttagggcctacggaaacgacgacctgctgaaggttggttacgaggttgggttcggagaaaagaactccctgggattcggcatggtgaaggtggaggggaacaagaccacaaaagaagctgaagagcaggaaaagatcaccttcaactctcgcgaggagctgaagaccggcgtgtga

SEQ ID NO: 19 - DNA, 564 bp, Pseudomonas aeruginosa
atggaccactatctggacatcagactgaggcccgatcctgagttccctcccgcccagctgatgagcgtgctgtttggcaagctgcatcaggctctggtcgcccaaggcggagacagaatcggcgtgtccttccccgacctggacgagtcccggagtcgcctgggcgagcggctgagaatccacgccagcgcagacgatctgcgcgccctgctggcccggccttggctggagggcctgcgggatcatctgcagtttggcgagcccgccgtggtgccacacccaacaccctaccgccaggtgagccgcgtgcaggccaagtcaaatcccgagagactgcggcggaggctgatgaggcgacatgatctgagcgaggaggaggccagaaagagaatccccgacacagtggccagagccctggatctgccatttgtgaccctgcggagccagagcactggccagcatttcagactgttcatcagacacgggcccctgcaggtgacagccgaggagggcggatttacatgctatggcctgtctaaaggcggcttcgtgccctggttctga

SEQ ID NO: 20 - DNA, 597 bp, Escherichia coli
atgtacctcagtaagatcatcatcgcccgcgcttggtcccgtgacctgtaccaactgcaccaagagctctggcacctcttccccaacaggccagatgccgctagagacttcctgttccacgtggagaagcgtaacacccccgaagggtgccacgtgctgttgcagagtgcccagatgccagtgagtaccgctgttgccactgtcatcaagactaaacaagttgaattccaactgcaagtgggcgtccctctgtatttccgcctcagggccaaccccatcaaaaccatcctggacaaccagaagcggctggatagcaaaggtaatatcaagagatgccgcgtgcctctgatcaaggaggccgagcagatcgcttggctgcaacgcaagctgggtaacgccgcgagagtggaagatgtgcacccaatctccgagcgcccgcagtatttctccggggaggggaagaacggcaaaattcagactgtctgcttcgagggggtgctcactattaacgacgcccctgctctgatcgacctcctgcagcagggcattgggcccgcgaagagcatgggatgcggattgttgagcctggcacccctg

SEQ ID NO: 21 - DNA, 636 bp, Thermus thermophilus
atgtggttgaccaaattggttctgaatcctgcgagccgcgcagcacggcgcgatttggctaacccttacgagatgcatcggactctttcaaaagcggttagcagggctttggaagaagggcgcgagcgccttttgtggaggctggagccagctcggggactggagccccctgtcgtcctggtgcagaccctcactgagcctgattggtccgtacttgatgaaggttacgcacaggtctttcctcctaagcctttccacccagcattgaagccgggccagcggctccgctttaggctccgggcgaatcccgccaaacggttggctgccaccggaaagcgagttgcgttgaaaacgcccgccgaaaaagtggcgtggcttgagaggcggctggaggagggtggttttcgactccttgaaggggaaaggggaccatgggtacagatacttcaagatacgttcctggaggtgcggagaaaaaaagacggagaagaggcaggcaagctgcttcaagtccaagccgtcttgttcgaggggagactcgaagttgttgatcctgagagagcacttgcgacactgagacgaggggtgggacctggtaaagcgctgggtcttggacttcttagtgttgcaccatga

SEQ ID NO: 22 - DNA, 29 bp, Pyrococcus furiosus
gttacaataagactaaatagaattgaaag

SEQ ID NO: 23 - DNA, 28 bp, Pseudomonas aeruginosa
gttcactgccgtataggcagctaagaaa

SEQ ID NO: 24 - DNA, 29 bp, Escherichia coli
gagttccccgcgccagcggggataaaccg

SEQ ID NO: 25 - DNA, 29 bp, Thermus thermophilus
gtagtccccacgcgtgtggggatggaccg